(19) United States
(12) Patent Application Publication
(10) Pub. No.: US 2024/0018523 A1
(43) Pub. Date: Jan. 18, 2024

(54) GALNAC COMPOSITIONS FOR IMPROVING SIRNA BIOAVAILABILITY

(71) Applicant: Empirico Inc., San Diego, CA (US)

(72) Inventors: Darren H. WAKEFIELD, Fitchburg, WI (US); David ROZEMA, Cross Plains, WI (US); Omri GOTTESMAN, San Diego, CA (US)

(21) Appl. No.: 18/037,960

(22) PCT Filed: Dec. 21, 2021
(86) PCT No.: PCT/US2021/064581

§ 371 Date: May 19, 2023

Related U.S. Application Data
(60) Provisional application No. 63/129,126, filed on Dec. 22, 2020.

(51) Int. Cl. C12N 15/113; A61P 1/16

(52) U.S. Cl. CPC C12N 15/1136; A61P 1/16; C12N 2310/351; C12N 2310/3515; C12N 2320/32; C12N 2310/14

(57) ABSTRACT

Provided herein, are compositions comprising GalNAc moieties that may be conjugated to an oligonucleotide. The oligonucleotide may be a small interfering RNA or an antisense oligonucleotide. Also provided herein are methods of treatment that include administering the composition to a subject.

### CROSS-REFERENCE

This application claims the benefit of U.S. Provisional Application No. 63/129,126, filed Dec. 22, 2020, which application is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 20, 2021, is named 54462-723_601_SL.txt and is 69,768 bytes in size.

### BACKGROUND

Cardiovascular, metabolic, and liver-related disorders are abundant, and may affect a wide variety of persons. Improved therapeutics are needed for treating these disorders.

### SUMMARY

Disclosed herein, in some embodiments, are compounds represented by Formula (I):

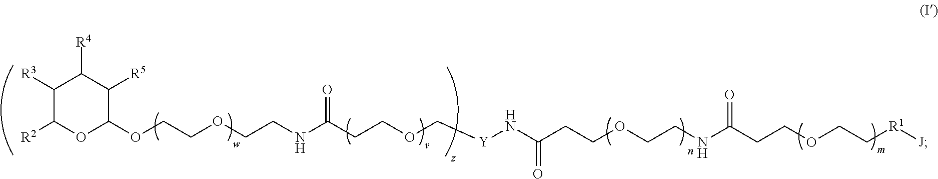

or a salt thereof, wherein J is an oligonucleotide; each w is independently selected from any value from 1 to 20; each v is independently selected from any value from 1 to 20; n is selected from any value from 1 to 20; m is selected from any value from 1 to 20; z is selected from any value from 1 to 3, wherein if z is 3, Y is C if z is 2, Y is CR⁶, or if z is 1, Y is C(R⁶)₂; R¹ is a linker selected from: —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—; each R² is independently selected from: C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; R³ and R⁴ are each independently selected from: —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; each R⁵ is independently selected from: —OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂; each R⁶ is independently selected from: hydrogen, halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and C₁₋₆ alkyl optionally substituted with one or more substituents independently selected from halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; each R⁷ is independently selected from: hydrogen, C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆haloalkyl. In some embodiments, each w is independently selected from any value from 1 to 10. In some embodiments, each w is independently selected from any value from 1 to 5. In some embodiments, each w is 1. In some embodiments, each v is independently selected from any value from 1 to 10. In some embodiments, each v is independently selected from any value from 1 to 5. In some embodiments, each v is 1. In some embodiments, n is selected from any value from 1 to 10. In some embodiments, n is selected from any value from 1 to 5. In some embodiments, n is 2. In some embodiments, m is selected from any value from 1 to 10. In some embodiments, m is selected from any value from 1 to 5. In some embodiments, m is 4. In some embodiments, z is 3 and Y is C. In some embodiments, R¹ is —OP(O)(OR⁷)O—. In some embodiments, R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, R² is selected from C₁₋₃ alkyl substituted with one or more substituents independently selected from —OR⁷, —OC(O)R⁷, —SR⁷, and —N(R⁷)₂. In some embodiments, R³ is selected from halogen, —OR⁷, —SR⁷, —N(R⁷)₂, —C(O)R⁷, —OC(O)R⁷, and —S(O)R⁷. In some embodiments, R³ is selected from —OR⁷, —SR⁷, —OC(O)R⁷, and —N(R⁷)₂. In some embodiments, R⁴ is selected from halogen, —OR⁷, —SR⁷, —N(R⁷)₂, —C(O)R⁷, —OC(O)R⁷, and —S(O)R⁷. In some embodiments, R⁴ is selected from —OR⁷, —SR⁷, —OC(O)R⁷, and —N(R⁷)₂. In some embodiments, R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, R⁵ is selected from —OC(O)R⁷ and —N(R⁷)C(O)R⁷. In some embodiments, each R⁷ is independently selected from: hydrogen, and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, the compound comprises:

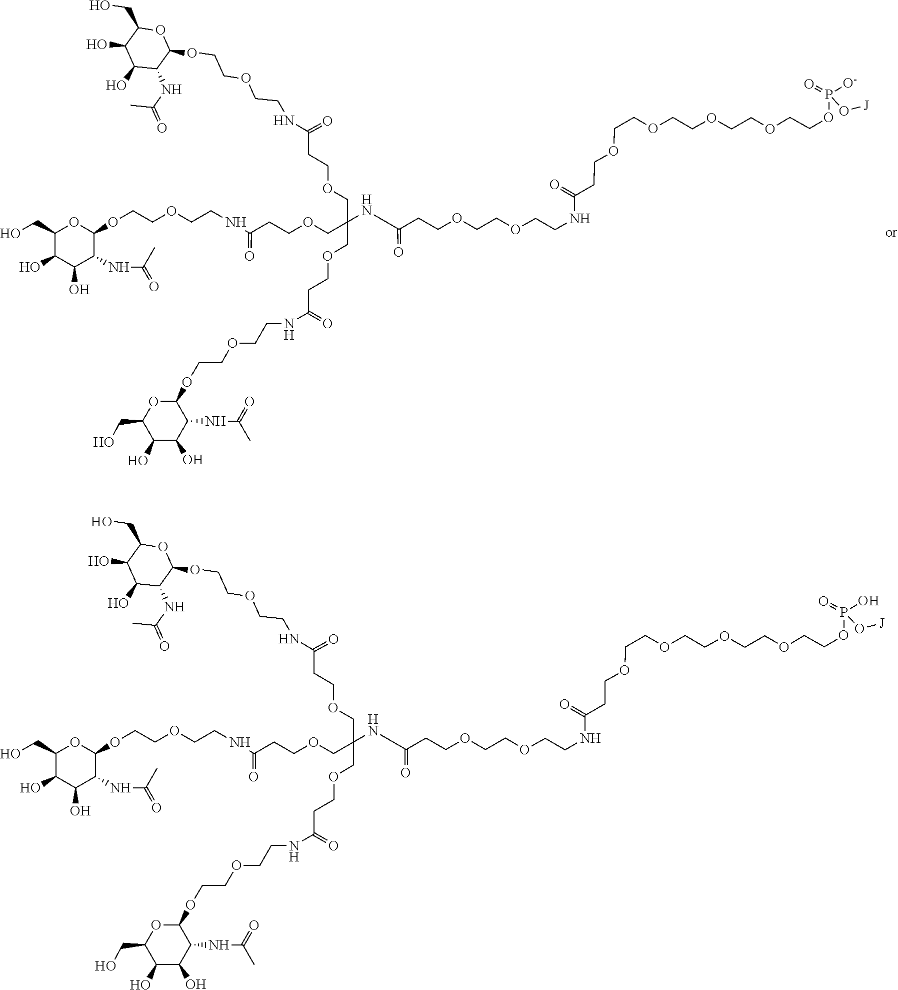

Some embodiments relate to a compound or salt represented by Formula (II):

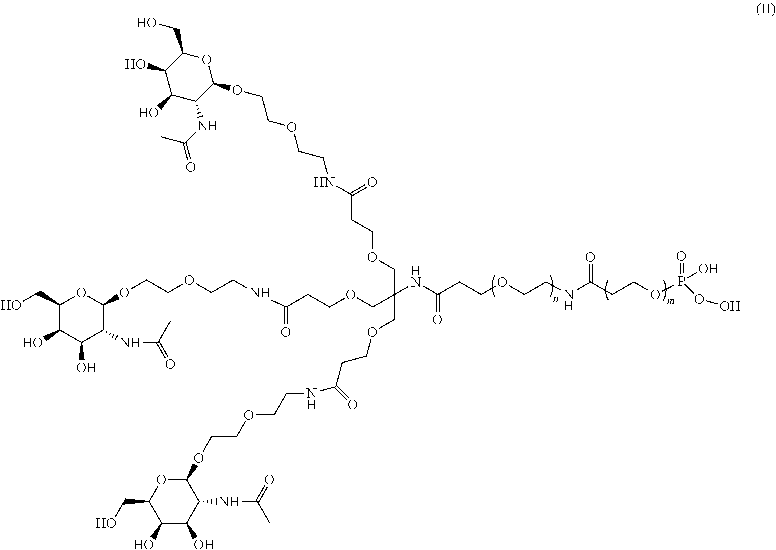

wherein J is an oligonucleotide; n is selected from any value from to 10; an m is selected from any value from 1 to 10. In some embodiments, n is 2. In some embodiments, m is 4. In some embodiments, the oligonucleotide (J) is attached at a 5′ end or a 3′ end of the oligonucleotide. In some embodiments, the oligonucleotide comprises DNA. In some embodiments, the oligonucleotide comprises RNA. In some embodiments, the oligonucleotide comprises one or more modified internucleoside linkages. In some embodiments, the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the oligonucleotide comprises 1, 2, 3, 4, 5, 6, 7, 8, 9, 10, 11, 12, 13, 14, 15, 16, 17, 18, 19, or 20 modified internucleoside linkages. In some embodiments, the oligonucleotide comprises one or more modified nucleosides. In some embodiments, the one or more modified nucleosides comprise a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof. In some embodiments, the one or more modified nucleosides comprise a 2′,4′ constrained ethyl nucleoside, a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, 2′-ara-F, 2′fluoro, or 2′ O-alkyl, or a combination thereof. In some embodiments, the oligonucleotide comprises 1, 2, 3, 4, 5, 6, 7, 8, 9, 10, 11, 12, 13, 14, 15, 16, 17, 18, 19, 20, 21, or more modified nucleosides. In some embodiments, the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof. In some embodiments, the oligonucleotide comprises an arginine-glycine-aspartic acid (RGD) peptide attached at a 3′ or 5′ terminus of the oligonucleotide. In some embodiments, the RGD peptide comprises Cyclo(-Arg-Gly-Asp-D-Phe-Cys), Cyclo(-Arg-Gly-Asp-D-Phe-Lys), Cyclo(-Arg-Gly-Asp-D-Phe-azido), an amino benzoic acid derived RGD, or a combination thereof. In some embodiments, the oligonucleotide comprises a small interfering RNA (siRNA) comprising a sense strand and an antisense strand. In some embodiments, the sense strand is 12-30 nucleosides in length. In some embodiments, the antisense strand is 12-30 nucleosides in length. In some embodiments, the sense strand and the antisense strand form a double-stranded RNA duplex. In some embodiments, a first base pair of the double-stranded RNA duplex is an AU base pair. In some embodiments, the sense strand or the antisense strand comprises a 3′ overhang. In some embodiments, the 3′ overhang comprises 1, 2, or more nucleosides. In some embodiments, the sense strand comprises any one of modification patterns 1 S to 6S, or 1 S #2 to 6S #2. In some embodiments, the antisense strand comprises any one of modification patterns 1AS to 9AS. In some embodiments, the oligonucleotide comprises an antisense oligonucleotide (ASO). In some embodiments, the ASO is 12-30 nucleosides in length. In some embodiments, the ASO comprises modification pattern ASOL. In some embodiments, the compound binds to an asialoglycoprotein receptor. In some embodiments, the compound targets a hepatocyte. In some embodiments, the compound, and a pharmaceutically acceptable carrier, excipient, or diluent. In some embodiments, the pharmaceutical composition is sterile. In some embodiments, the pharmaceutical composition comprises a pharmaceutically acceptable carrier. In some embodiments, the pharmaceutically acceptable carrier comprises water, a buffer, or a saline solution. In some embodiments, the oligonucleotide targets a target mRNA and when administered to a subject in an effective amount decreases the target mRNA or a target protein by at least 10%. Some embodiments relate to a method of decreasing a target mRNA or target protein in a subject in need thereof, comprising administering an effective amount of the pharmaceutical composition to the subject. In some embodiments, the effective amount decreases a measurement of the target mRNA or target protein in the subject, relative to a baseline target mRNA or target protein measurement. In some embodiments, the effective amount treats a disorder in the subject. In some embodiments, the effective amount decreases a measurement of a symptom or parameter related to the disorder in the subject, relative to a baseline symptom or parameter measurement. In some embodiments, the disorder comprises a metabolic disorder. In some embodiments, the disorder comprises a liver disorder.

### DETAILED DESCRIPTION

N-Acetylgalactosamine (GalNAc), is an amino sugar derivative of galactose. GalNAc and GalNAc-containing moieties may bind lectins such as asialoglycoprotein receptors. These receptors may be included on hepatocytes. Thus, GalNAc may target an oligonucleotide to a hepatocyte or to the liver.

Provided herein, are GalNAc moieties. These GalNAc moieties may be conjugated to an oligonucleotide such as a small interfering RNA (siRNA) or antisense oligonucleotide (ASO). The oligonucleotide conjugated to the GalNAc moiety may be administered to a subject, targeted to a liver or hepatocyte, or used to treat a liver related disorder in the subject.

### I. COMPOSITIONS

Provided herein, in some embodiments, are compositions comprising an oligonucleotide and an N-Acetylgalactosamine (GalNAc) moiety. In some embodiments, the composition comprises an oligonucleotide. The oligonucleotide may inhibit a target gene or oligonucleotide. The oligonucleotide may bind a target oligonucleotide. In some embodiments, the composition is used in a method described herein.

Provided herein, in some embodiments, are compounds comprising an oligonucleotide and a GalNAc moiety. In some embodiments, the compound comprises an oligonucleotide. The oligonucleotide may bind to a target oligonucleotide. In some embodiments, the compound is used in a method described herein. In some embodiments, the compound is included in a composition described herein.

The oligonucleotide of the compound or composition described herein may comprise a small interfering RNA (siRNA) or an antisense oligonucleotide (ASO).

Some embodiments include a composition comprising a GalNAc moiety, and an oligonucleotide that when administered to a subject in an effective amount decreases a target mRNA or protein level in a cell, fluid or tissue of the subject. Some embodiments include a composition comprising a GalNAc moiety, and an oligonucleotide that when administered to a subject in an effective amount decreases a target mRNA or protein level in liver tis sue or in a hepatocyte. In some embodiments, the composition comprises a GalNAc moiety, and an oligonucleotide that when administered to a subject in an effective amount decreases levels of a target e.g. mRNA in a cell or tissue. In some embodiments, the cell is a hepatocyte. In some embodiments, the tissue is liver tissue. Some embodiments include a composition comprising a GalNAc moiety, and an oligonucleotide that when administered to a subject in an effective amount decreases a target mRNA level in liver tissue. Some embodiments include a composition comprising a GalNAc moiety, and an oligonucleotide that when administered to a subject in an effective amount decreases a target mRNA level in a hepatocyte.

In some embodiments, the decrease in the target oligonucleotide level is specific to a hepatocyte in relation to another cell type. In some embodiments, the decrease in the target RNA level is specific to a hepatocyte in relation to another cell type. In some embodiments, the decrease in the target mRNA level is specific to a hepatocyte in relation to another cell type. In some embodiments, the decrease in the target protein level is specific to a hepatocyte in relation to another cell type. In some embodiments, the decrease in the target oligonucleotide level is specific to liver tissue in relation to another tissue type. In some embodiments, the decrease in the target RNA level is specific to liver in relation to another tissue type. In some embodiments, the decrease in the target mRNA level is specific to liver in relation to another tissue type. In some embodiments, the decrease in the target protein level is specific to liver in relation to another tissue type.

In some embodiments, the composition comprises a GalNAc moiety, and an oligonucleotide that binds to a target oligonucleotide, which when administered to a subject in an effective amount decreases the target oligonucleotide levels in a cell or tissue. In some embodiments, the target oligonucleotide levels are decreased by about 2.5% or more, about 5% or more, or about 7.5% or more, as compared to prior to administration. In some embodiments, the target oligonucleotide levels are decreased by about 10% or more, as compared to prior to administration. In some embodiments, the target oligonucleotide levels are decreased by about 20% or more, about 30% or more, about 40% or more, about 50% or more, about 60% or more, about 70% or more, about 80% or more, about 90% or more, or about 100%, as compared to prior to administration. In some embodiments, the target oligonucleotide levels are decreased by no more than about 2.5%, no more than about 5%, or no more than about 7.5%, as compared to prior to administration. In some embodiments, the target oligonucleotide levels are decreased by no more than about 10%, as compared to prior to administration. In some embodiments, the target oligonucleotide levels are decreased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the target oligonucleotide levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a GalNAc moiety, and an oligonucleotide that binds to a target mRNA, which when administered to a subject in an effective amount decreases the target mRNA levels in a cell or tissue. In some embodiments, the target mRNA levels are decreased by about 2.5% or more, about 5% or more, or about 7.5% or more, as compared to prior to administration. In some embodiments, the target mRNA levels are decreased by about 10% or more, as compared to prior to administration. In some embodiments, the target mRNA levels are decreased by about 20% or more, about 300% or more, about 40% or more, about 50% or more, about 60% or more, about 70% or more, about 80% or more, about 90% or more, or about 100%, as compared to prior to administration. In some embodiments, the target mRNA levels are decreased by no more than about 2.5%, no more than about 5%, or no more than about 7.5%, as compared to prior to administration. In some embodiments, the target mRNA levels are decreased by no more than about 10%, as compared to prior to administration. In some embodiments, the target mRNA levels are decreased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the target mRNA levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by arange defined by any of the two aforementioned percentages.

In some embodiments, the composition comprises a GalNAc moiety, and an oligonucleotide that binds to an oligonucleotide encoding a target protein, which when the composition is administered to a subject in an effective amount decreases the target protein levels in a cell or tissue. In some embodiments, the cell is a hepatocyte. In some embodiments, the tissue is liver tissue. In some embodiments, the target protein levels are decreased by about 2.5% or more, about 5% or more, or about 7.5% or more, as compared to prior to administration. In some embodiments, the target protein levels are decreased by about 10% or more, as compared to prior to administration. In some embodiments, the target protein levels are decreased by about 20% or more, about 30% or more, about 40% or more, about 50% or more, about 60% or more, about 70% or more, about 80% or more, about 90% or more, or about 100%, as compared to prior to administration. In some embodiments, the target protein levels are decreased by no more than about 2.5%, no more than about 5%, or no more than about 7.5%, as compared to prior to administration. In some embodiments, the target protein levels are decreased by no more than about 10%, as compared to prior to administration. In some embodiments, the target protein levels are decreased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the target protein levels are decreased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a GalNAc moiety, and an oligonucleotide that binds to a target oligonucleotide (e.g. mRNA) and when administered to a subject in an effective amount decreases an adverse phenotype (e.g. a symptom of a disorder associated with the target oligonucleotide). In some embodiments, the adverse phenotype is decreased by about 2.5% or more, about 5% or more, or about 7.5% or more, as compared to prior to administration. In some embodiments, the adverse phenotype is decreased by about 10% or more, as compared to prior to administration. In some embodiments, the adverse phenotype is decreased by about 20% or more, about 30% or more, about 40% or more, about 50% or more, about 60% or more, about 70% or more, about 80% or more, about 90% or more, or about 100%, as compared to prior to administration. In some embodiments, the adverse phenotype is decreased by no more than about 2.5%, no more than about 5%, or no more than about 7.5%, as compared to prior to administration. In some embodiments, the adverse phenotype is decreased by no more than about 10%, as compared to prior to administration. In some embodiments, the adverse phenotype is decreased by no more than about 20%, no more than about 30%, no more than about 40%, no more than about 50%, no more than about 60%, no more than about 70%, no more than about 80%, no more than about 90%, or no more than about 100%, as compared to prior to administration. In some embodiments, the adverse phenotype is decreased by 2.5%, 5%, 7.5% 10%, 15%, 20%, 25%, 30%, 35%, 40%, 45%, 50%, 55%, 60%, 65%, 70%, 75%, 80%, 85%, 90%, 95%, or 100%, or by a range defined by any of the two aforementioned percentages.

In some embodiments, the composition comprises a GalNAc moiety, and an oligonucleotide that binds to a target oligonucleotide (e.g. mRNA) and when administered to a subject in an effective amount increases a protective phenotype (e.g. protective against a disorder). In some embodiments, the protective phenotype is increased by about 2.5% or m ore, about 5% or more, or about 7.5% or more, as compared to prior to administration. In some embodiments, the protective phenotype is increased by about 10% or more, as compared to prior to administration. In some embodiments, the protective phenotype is increased by about 20% or more, about 30% or more, about 40% or more, about 50% or more, about 60% or more, about 70% or more, about 80% or more, about 90% or more, or about 100% or more, as compared to prior to administration. In some embodiments, the protective phenotype is increased by about 200% or more, about 300% or more, about 400% or more, about 500% or more, about 600% or more, about 700% or more, about 800% or more, about 900% or more, or about 1000% or more, as compared to prior to administration. In some embodiments, the protective phenotype is increased by no more than about 2.5%, no more than about 5%, or no more than about 7.5%, as compared to prior to administration. In some embodiments, the protective phenotype is increased by no more than about 10%, as compared to prior to administration. In some embodiments, the protective phenotype is increased by no more than about 20%, no more than about 300%, no more than about 40%, no more than about 50%, no more than about 60%, no more than about 70%, no more than about 80%, no more than about 90%, or no more than about 100%, as compared to prior to administration. In some embodiments, the protective phenotype is increased by no more than about 200%, no more than about 300%, no more than about 400%, no more than about 500%, no more than about 600%, no more than about 700%, no more than about 800%, no more than about 900%, or no more than about 1000%, as compared to prior to administration. In some embodiments, the protective phenotype is increased by 2.5%, 5%, 7.5%, 10%, 15%, 20%, 25%, 30%, 35%, 40%, 45%, 50%, 55%, 60%, 65%, 70%, 75%, 80%, 85%, 90%, 95%, 100%, 150%, 200%, 250%, 300%, 400%, 500%, 600%, 700%, 800%, 900%, or 1000%, or by a range defined by any of the two aforementioned percentages.

A. GalNAc Moieties and Compounds

Provided herein, in some embodiments, are compositions comprising a GalNAc moiety. Provided herein, in some embodiments, are compositions comprising a GalNAc moiety and an oligonucleotide. In some embodiments, a composition comprising GalNAc moiety and an oligonucleotide is described by a compound of Formula (I), Formula (II), or Formula (III). In some embodiments the oligonucleotide of Formula (I), Formula (II), or Formula (III) is J. In some embodiments the GalNAc moiety of Formula (I), Formula (II), or Formula (III) is the molecular moiety bound to J. The oligonucleotide may comprise a small interfering RNA (siRNA) or an antisense oligonucleotide (ASO).

Provided herein, in some embodiments, is a compound represented by Formula (I):

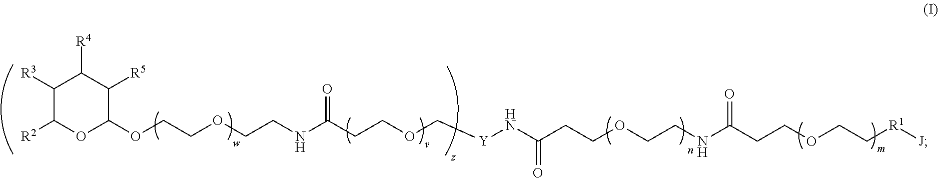

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
if z is 3, Y is C
if z is 2, Y is CR⁶, or
if z is 1, Y is C(R⁶)₂;
   R¹ is a linker selected from:
—O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
   each R² is independently selected from:
C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   R³ and R⁴ are each independently selected from:
—OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   each R⁵ is independently selected from:
—OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
   each R⁶ is independently selected from:
hydrogen;
halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
C₁₋₆ alkyl optionally substituted with one or more substituents independently selected from halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; each R⁷ is independently selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆haloalkyl.

In some embodiments, each w is independently selected from any value from 1 to 20. In some embodiments, each w is independently selected from any value from 1 to 15. In some embodiments, each w is independently selected from any value from 1 to 10. In some embodiments, each w is independently selected from any value from 1 to 5. In some embodiments, each w is independently selected from any value from 1 to 4. In some embodiments, each w is independently selected from any value from 1 to 3. In some embodiments, each w is independently selected from any value from 1 to 2. In some embodiments, each w is independently 1. In some embodiments, w is 1, 2, 3, 4, 5, 6, 7, 8, 9, 10, 11, 12, 13, 14, 15, 16, 17, 18, 19, or 20.

In some embodiments, each v is independently selected from any value from 1 to 20. In some embodiments, each v is independently selected from any value from 1 to 15. In some embodiments, each v is independently selected from any value from 1 to 10. In some embodiments, each v is independently selected from any value from 1 to 5. In some embodiments, each v is independently selected from any value from 1 to 4. In some embodiments, each v is independently selected from any value from 1 to 3. In some embodiments, each v is independently selected from any value from 1 to 2. In some embodiments, each v is independently 1. In some embodiments, v is 1, 2, 3, 4, 5, 6, 7, 8, 9, 10, 11, 12, 13, 14, 15, 16, 17, 18, 19, or 20.

In some embodiments, n is selected from any value from 1 to 20. In some embodiments, n is selected from any value from 1 to 15. In some embodiments, n is selected from any value from 1 to 10. In some embodiments, n is selected from any value from 1 to 9. In some embodiments, n is selected from any value from 1 to 8. In some embodiments, n is selected from any value from 1 to 7. In some embodiments, n is selected from any value from 1 to 6. In some embodiments, n is selected from any value from 1 to 5. In some embodiments, n is selected from any value from 1 to 4. In some embodiments, n is selected from any value from 2 to 4. In some embodiments, n is selected from any value from 1 to 3. In some embodiments, n is 2 or 3. In some embodiments, n is 3. In some embodiments, n is selected from any value from 1 to 2. In some embodiments, n is 2. In some embodiments, n is 1. In some embodiments, n is 1, 2, 3, 4, 5, 6, 7, 8, 9, 10, 11, 12, 13, 14, 15, 16, 17, 18, 19, or 20.

In some embodiments, m is selected from any value from 1 to 20. In some embodiments, m is selected from any value from 1 to 15. In some embodiments, m is selected from any value from 1 to 10. In some embodiments, m is selected from any value from 1 to 9. In some embodiments, m is selected from any value from 1 to 8. In some embodiments, m is selected from any value from 1 to 7. In some embodiments, m is selected from any value from 3 to 7. In some embodiments, m is selected from any value from 1 to 6. In some embodiments, m is selected from any value from 2 to 6. In some embodiments, m is selected from any value from 3 to 6. In some embodiments, m is selected from any value from 4 to 6. In some embodiments, m is 6. In some embodiments, m is selected from any value from 1 to 5. In some embodiments, m is selected from any value from 3 to 5. In some embodiments, m is 5. In some embodiments, m is 4 or 5. In some embodiments, m is selected from any value from 1 to 4. In some embodiments, m is 4. In some embodiments, m is 3 or 4. In some embodiments, m is selected from any value from 2 to 4. In some embodiments, m is selected from any value from 1 to 3. In some embodiments, m is 3. In some embodiments, m is selected from any value from 1 to 2. In some embodiments, m is 2. In some embodiments, m is 1. In some embodiments, m is 1, 2, 3, 4, 5, 6, 7, 8, 9, 10, 11, 12, 13, 14, 15, 16, 17, 18, 19, or 20.

In some embodiments, z is selected from any value from 1 to 3. In some embodiments, z is selected from any value from 2 to 4. In some embodiments, z is selected from any value from 1 to 5. In some embodiments, z is 5. In some embodiments, z is 4. In some embodiments, z is 3. In some embodiments, z is 2. In some embodiments, z is 1. In some embodiments, z is 3 and Y is C. In some embodiments, z is 2 and Y is CR⁶. In some embodiments, z is 1 and Y is C(R⁶)₂.

In some embodiments, R¹ is a linker selected from —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—. In some embodiments, R¹ is a linker selected from —O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—. In some embodiments, R¹ is a linker selected from —OP(O)(OH)O—, —SP(O)(OH)O—, —OP(S)(OH)O—, —OP(O)(SH)O—, —OP(O)(OH)S—, —OP(O)(O⁻)O—, —SP(O)(O⁻)O—, —OP(S)(O⁻)O—, —OP(O)(S⁻)O—, and —OP(O)(O⁻)S—. In some embodiments, R¹ is a selected from —OP(O)(OR⁷)O—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—. In some embodiments, R¹ is selected from —OP(O)(OR⁷)O—, —OP(OR⁷)N(R⁷)—, and —OPN(R⁷)₂O—. In some embodiments, R¹ is —OP(O)(OH)O—, —OP(O)(OCH₂CH₃)O—, —OP(OCH₂CH₂CN)N(CH(CH₃)₂)—, or —OPN(CH(CH₃)₂)₂O—. In some embodiments, R¹ is selected from —OP(O)(OH)O— and OP(O)(O⁻)O—. In some embodiments, R¹ comprises —O— or —S—. In some embodiments, R¹ comprises —O—. In some embodiments, R¹ comprises —S—. In some embodiments, R¹ is a linker selected from —O— or —S—. In some embodiments, R¹ is —O—. In some embodiments, R¹ is —S—.

In some embodiments, each R² is independently selected from C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R² is independently selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, each R² is independently selected from C₁₋₃ alkyl substituted with one or more substituents independently selected from —OR⁷, —OC(O)R⁷, —SR⁷, and —N(R⁷)₂. In some embodiments, each R² is independently selected from C₁ alkyl substituted with one or more substituents independently selected from —OR⁷ and —OC(O)R⁷. In some embodiments, each R² is independently selected from —CH₂OH and —CH₂OC(O)CH₃.

In some embodiments, each R³ is independently selected from —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R³ is independently selected from halogen, —OR⁷, —SR⁷, —N(R⁷)₂, —C(O)R⁷, —OC(O)R⁷, and —S(O)R⁷. In some embodiments, each R³ is independently selected from —OR⁷, —OC(O)R⁷, —SR⁷, and —N(R⁷)₂. In some embodiments, each R³ is independently selected from —OR⁷ and —OC(O)R⁷. In some embodiments, R³ is independently selected from —OH and —OC(O)CH₃.

In some embodiments, each R⁴ is independently selected from —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R⁴ is independently selected from halogen, —OR⁷, —SR⁷, —N(R⁷)₂, —C(O)R⁷, —OC(O)R⁷, and —S(O)R⁷. In some embodiments, each R⁴ is independently selected from —OR⁷, —OC(O)R⁷, —SR⁷, and —N(R⁷)₂. In some embodiments, each R⁴ is independently selected from —OR⁷ and —OC(O)R⁷. In some embodiments, R⁴ is independently selected from —OH and —OC(O)CH₃.

In some embodiments, each R⁵ is independently selected from—OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂. In some embodiments, each R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, each R⁵ is independently selected from —OC(O)R⁷ and —N(R⁷)C(O)R⁷. In some embodiments, each R⁵ is independently selected from —N(H)C(O)CH₃.

In some embodiments, each R⁶ is independently selected from hydrogen, halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and C₁₋₆ alkyl optionally substituted with one or more substituents independently selected from halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R⁶ is independently selected from hydrogen, halogen, —CN—OR⁷, —SR⁷, —N(R⁷)₂, and C₁₋₆ alkyl optionally substituted with one or more substituents independently selected from halogen, —OR⁷, —SR⁷, and —N(R⁷)₂. In some embodiments, each R⁶ is independently selected from hydrogen, halogen, —CN, —OH, —SH, and —NH₂.

In some embodiments, each R⁷ is independently selected from: hydrogen; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl.

In some embodiments, each R⁷ is independently selected from: hydrogen; and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from hydrogen. In some embodiments, each R⁷ is independently selected from C₁₋₃ alkyl optionally substituted with one or more substituents independently selected from halogen, —CN, —OH, —SH, —NO₂, —NH₂, —O—C₁₋₆alkyl, —S—C₁₋₆ alkyl, —N(C₁₋₆ alkyl)₂, and —NH(C₁₋₆ alkyl).

In some embodiments, a compound of Formula (I) is selected from

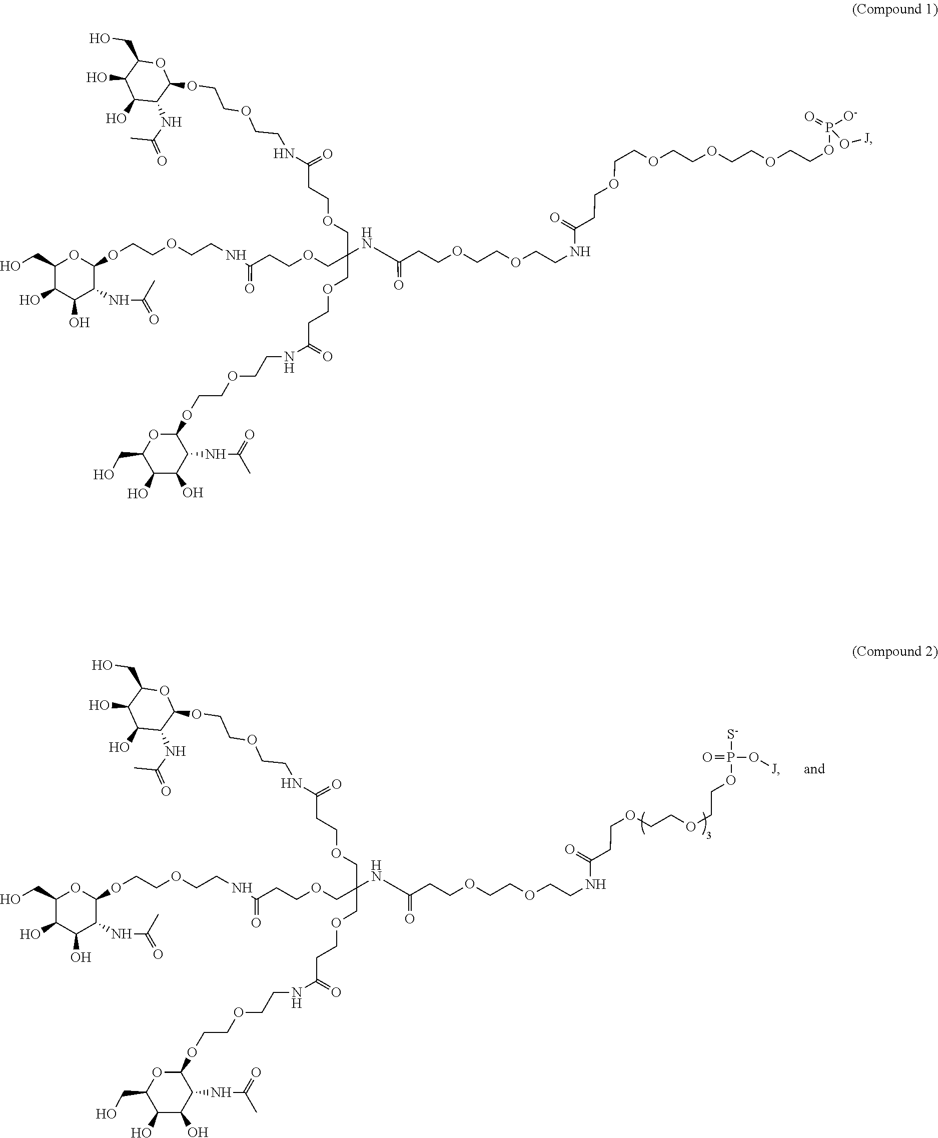

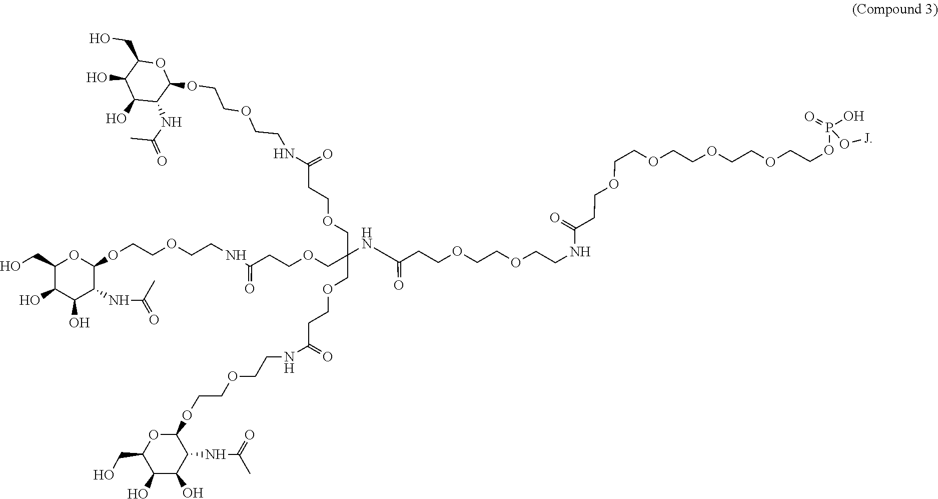

In some embodiments, the compound of Formula (I) comprises Compound 1. In some embodiments, the compound of Formula (I) comprises Compound 2. In some embodiments, the compound of Formula (I) comprises Compound 3.

In some embodiments, the GalNAc moiety comprises Compound 1. In some embodiments, the GalNAc moiety comprises Compound 2. In some embodiments, the GalNAc moiety comprises Compound 3.

Provided herein, in some embodiments, is a compound represented by Formula (II):

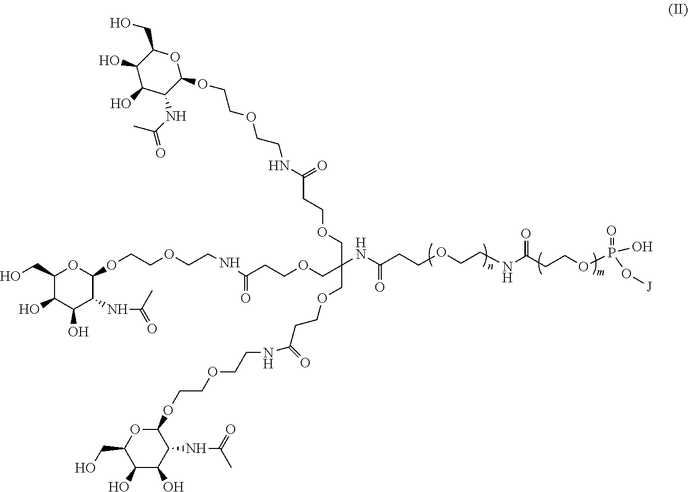

or a salt thereof, wherein
J is an oligonucleotide;
n is selected from any value from 1 to 10; and
m is selected from any value from 1 to 10.

In some embodiments, n is selected from any value from 1 to 10. In some embodiments, n is selected from any value from 1 to 9. In some embodiments, n is selected from any value from 1 to 8. In some embodiments, n is selected from any value from 1 to 7. In some embodiments, n is selected from any value from 1 to 6. In some embodiments, n is selected from any value from 1 to 5. In some embodiments, n is selected from any value from 1 to 4. In some embodiments, n is selected from any value from 2 to 4. In some embodiments, n is selected from any value from 1 to 3. In some embodiments, n is 2 or 3. In some embodiments, n is 3. In some embodiments, n is selected from any value from 1 to 2. In some embodiments, n is 2. In some embodiments, n is 1. In some embodiments, n is 1, 2, 3, 4, 5, 6, 7, 8, 9, or 10.

In some embodiments, m is selected from any value from 1 to 10. In some embodiments, m is selected from any value from 1 to 9. In some embodiments, m is selected from any value from 1 to 8. In some embodiments, m is selected from any value from 1 to 7. In some embodiments, m is selected from any value from 3 to 7. In some embodiments, m is selected from any value from 1 to 6. In some embodiments, m is selected from any value from 2 to 6. In some embodiments, m is selected from any value from 3 to 6. In some embodiments, m is selected from any value from 4 to 6. In some embodiments, m is 6. In some embodiments, m is selected from any value from 1 to 5. In some embodiments, m is selected from any value from 3 to 5. In some embodiments, m is 5. In some embodiments, m is 4 or 5. In some embodiments, m is selected from any value from 1 to 4. In some embodiments, m is 4. In some embodiments, m is 3 or 4. In some embodiments, m is selected from any value from 2 to 4. In some embodiments, m is selected from any value from 1 to 3. In some embodiments, m is 3. In some embodiments, m is selected from any value from 1 to 2. In some embodiments, m is 2. In some embodiments, m is 1. In some embodiments, m is selected from any value from 4 to 6. In some embodiments, m is 1, 2, 3, 4, 5, 6, 7, 8, 9, or 10.

In some embodiments, the phosphate is deprotonated to form a salt of Formula (II). In some embodiments, the cation is a metal ion such as a metal cation. Non limiting examples of metal cations include Na⁺, K⁺, Mg²⁺, and Ca²⁺. In some embodiments, the metal cation comprises Na⁺. In some embodiments, the metal cation comprises K⁺. In some embodiments, the metal cation comprises Mg²⁺. In some embodiments, the metal cation comprises Ca²⁺. In some embodiments, the cation is an organic or inorganic small molecule. In some embodiments, the cation is a positively charged nucleic acid present in the oligonucleotide.

In some embodiments, the compound of Formula (II) comprises Compound 1. In some embodiments, the compound of Formula (II) comprises Compound 2. In some embodiments, the compound of Formula (II) comprises Compound 3.

Provided herein, in some embodiments, is a compound represented by Formula (III):

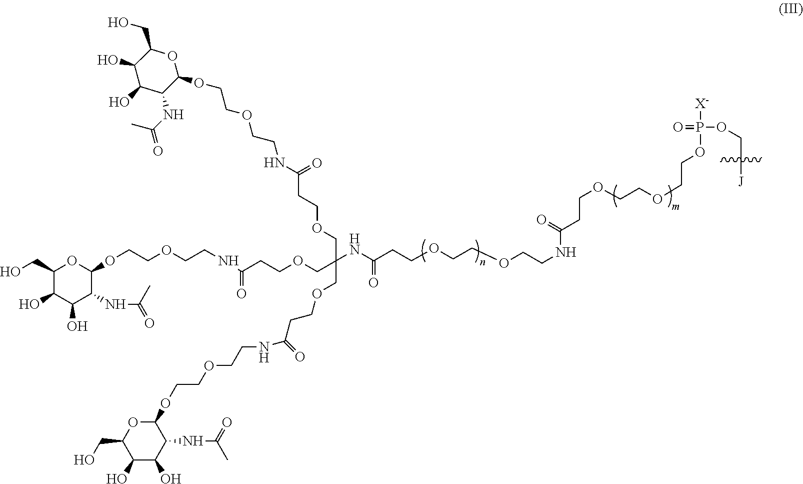

or a salt thereof, wherein
J is an oligonucleotide;
X is an oxygen (O) or sulfur (S);
n is selected from any value from 1 to 10; and
m is selected from any value from 1 to 10.

In some embodiments, Xis O. In some embodiments, S.

In some embodiments, n is selected from any value from 1 to 10. In some embodiments, n is selected from any value from 1 to 9. In some embodiments, n is selected from any value from 1 to 8. In some embodiments, n is selected from any value from 1 to 7. In some embodiments, n is selected from any value from 1 to 6. In some embodiments, n is selected from any value from 1 to 5. In some embodiments, n is selected from any value from 1 to 4. In some embodiments, n is selected from any value from 2 to 4. In some embodiments, n is selected from any value from 1 to 3. In some embodiments, n is 2 or 3. In some embodiments, n is 3. In some embodiments, n is selected from any value from 1 to 2. In some embodiments, n is 2. In some embodiments, n is 1. In some embodiments, n is 1, 2, 3, 4, 5, 6, 7, 8, 9, or 10.

In some embodiments, m is selected from any value from 1 to 10. In some embodiments, m is selected from any value from 1 to 9. In some embodiments, m is selected from any value from 1 to 8. In some embodiments, m is selected from any value from 1 to 7. In some embodiments, m is selected from any value from 3 to 7. In some embodiments, m is selected from any value from 1 to 6. In some embodiments, m is selected from any value from 2 to 6. In some embodiments, m is selected from any value from 3 to 6. In some embodiments, m is selected from any value from 4 to 6. In some embodiments, m is 6. In some embodiments, m is selected from any value from 1 to 5. In some embodiments, m is selected from any value from 3 to 5. In some embodiments, m is 4 or 5. In some embodiments, m is 5. In some embodiments, m is selected from any value from 1 to 4. In some embodiments, m is 4. In some embodiments, m is 3 or 4. In some embodiments, m is selected from any value from 2 to 4. In some embodiments, m is selected from any value from 1 to 3. In some embodiments, m is 3. In some embodiments, m is selected from any value from 1 to 2. In some embodiments, m is 2. In some embodiments, m is 1. In some embodiments, m is 1, 2, 3, 4, 5, 6, 7, 8, 9, or 10.

In some embodiments, the compound of Formula (III) comprises Compound 1. In some embodiments, the compound of Formula (III) comprises Compound 2. In some embodiments, the compound of Formula (III) comprises Compound 3.

In some embodiments, compositions described herein comprise a GalNAc compound. In some embodiments, a GalNAc compound describes a compound of Formula (A) or Formula (B). In some embodiments, compositions described herein comprise a GalNAc synthesis compound, such as a synthesis compound of Formula (C).

In some embodiments, the compound of Formula (I), (II), or (III) binds to a lectin. In some embodiments, the compound binds to an asialoglycoprotein receptor. In some embodiments, the compound binds to a liver cell receptor. In some embodiments, the compound binds to a hepatocyte receptor. In some embodiments, the compound targets a liver cell.

Provided herein, in some embodiments, is a compound represented by Formula (A):

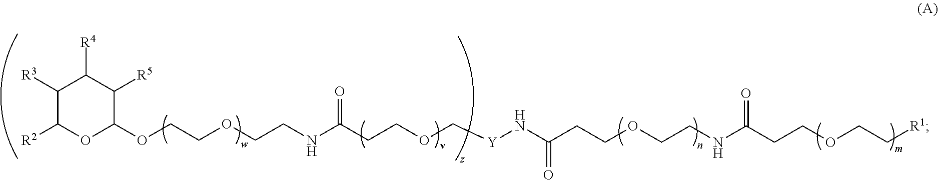

or a salt thereof, wherein
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
if z is 3, Y is C
if z is 2, Y is CR⁶, or
if z is 1, Y is C(R⁶)₂;
   R¹ is a selected from:
—OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, —S(O)R⁷, —S(O)₂R⁷, —OS(O)₂R⁷, —OP(O)(OR⁷)₂, —OP(O)(OR⁷)N(R⁷)₂, —OP(O)(N(R⁷)₂)₂, —OP(OR⁷)₂, —OP(OR⁷)N(R⁷)₂, and —OP(N(R⁷)₂)₂;
   each R² is independently selected from:
C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   R³ and R⁴ are each independently selected from:
—OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   each R⁵ is independently selected from:
—OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
   each R⁶ is independently selected from:
hydrogen;
halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
C₁₋₆ alkyl optionally substituted with one or more substituents independently selected from halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   each R⁷ is independently selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆haloalkyl.

In some embodiments, each w is independently selected from any value from 1 to 20. In some embodiments, each w is independently selected from any value from 1 to 15. In some embodiments, each w is independently selected from any value from 1 to 10. In some embodiments, each w is independently selected from any value from 1 to 5. In some embodiments, each w is independently selected from any value from 1 to 4. In some embodiments, each w is independently selected from any value from 1 to 3. In some embodiments, each w is independently selected from any value from 1 to 2. In some embodiments, W is 1. In some embodiments, w is 1, 2, 3, 4, 5, 6, 7, 8, 9, 10, 11, 12, 13, 14, 15, 16, 17, 18, 19, or 20.

In some embodiments, each v is independently selected from any value from 1 to 20. In some embodiments, each v is independently selected from any value from 1 to 15. In some embodiments, each v is independently selected from any value from 1 to 10. In some embodiments, each v is independently selected from any value from 1 to 5. In some embodiments, each v is independently selected from any value from 1 to 4. In some embodiments, each v is independently selected from any value from 1 to 3. In some embodiments, each v is independently selected from any value from 1 to 2. In some embodiments, each v is independently 1. In some embodiments, v is 1, 2, 3, 4, 5, 6, 7, 8, 9, 10, 11, 12, 13, 14, 15, 16, 17, 18, 19, or 20.

In some embodiments, n is selected from any value from 1 to 20. In some embodiments, n is selected from any value from 1 to 15. In some embodiments, n is selected from any value from 1 to 10. In some embodiments, n is selected from any value from 1 to 9. In some embodiments, n is selected from any value from 1 to 8. In some embodiments, n is selected from any value from 1 to 7. In some embodiments, n is selected from any value from 1 to 6. In some embodiments, n is selected from any value from 1 to 5. In some embodiments, n is selected from any value from 1 to 4. In some embodiments, n is selected from any value from 2 to 4. In some embodiments, n is selected from any value from 1 to 3. In some embodiments, n is 2 or 3. In some embodiments, n is 3. In some embodiments, n is selected from any value from 1 to 2. In some embodiments, n is 2. In some embodiments, n is 1. In some embodiments, n is 1, 2, 3, 4, 5, 6, 7, 8, 9, 10, 11, 12, 13, 14, 15, 16, 17, 18, 19, or 20.

In some embodiments, m is selected from any value from 1 to 20. In some embodiments, m is selected from any value from 1 to 15. In some embodiments, m is selected from any value from 1 to 10. In some embodiments, m is selected from any value from 1 to 9. In some embodiments, m is selected from any value from 1 to 8. In some embodiments, m is selected from any value from 1 to 7. In some embodiments, m is selected from any value from 3 to 7. In some embodiments, m is selected from any value from 1 to 6. In some embodiments, m is selected from any value from 2 to 6. In some embodiments, m is selected from any value from 3 to 6. In some embodiments, m is selected from any value from 4 to 6. In some embodiments, m is 6. In some embodiments, m is selected from any value from 1 to 5. In some embodiments, m is selected from any value from 3 to 5. In some embodiments, m is 5. In some embodiments, m is 4 or 5. In some embodiments, m is selected from any value from 1 to 4. In some embodiments, m is 4. In some embodiments, m is 3 or 4. In some embodiments, m is selected from any value from 2 to 4. In some embodiments, m is selected from any value from 1 to 3. In some embodiments, m is 3. In some embodiments, m is selected from any value from 1 to 2. In some embodiments, m is 2. In some embodiments, m is 1. In some embodiments, m is 1, 2, 3, 4, 5, 6, 7, 8, 9, 10, 11, 12, 13, 14, 15, 16, 17, 18, 19, or 20.

In some embodiments, z is selected from any value from 1 to 3. In some embodiments, z is selected from any value from 2 to 4. In some embodiments, z is selected from any value from 1 to 5. In some embodiments, z is 5. In some embodiments, z is 4. In some embodiments, z is 3. In some embodiments, z is 2. In some embodiments, z is 1. In some embodiments, z is 3 and Y is C. In some embodiments, z is 2 and Y is CR⁶. In some embodiments, z is 1 and Y is C(R⁶)₂.

In some embodiments, R¹ is a selected from —OP(O)(OR⁷)₂, —OP(O)(OR⁷)N(R⁷)₂, —OP(O)(N(R⁷)₂)₂, —OP(OR⁷)₂, —OP(OR⁷)N(R⁷)₂, and —OP((NR⁷)₂)₂. In some embodiments, R¹ is a selected from —OP(O)(OR⁷)₂ and —OP(OR⁷)N(R⁷)₂. In some embodiments, R¹ is selected from —OP(O)(OCH₂CH₃)OH and —OP(OCH₂CH₂CN)N(CH(CH₃)₂)₂.

In some embodiments, each R² is independently selected from C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R² is independently selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷. In some embodiments, each R² is independently selected from C₁₋₃ alkyl substituted with one or more substituents independently selected from —OR⁷, —OC(O)R⁷, —SR⁷, and —N(R⁷)₂. In some embodiments, each R² is independently selected from C₁ alkyl substituted with one or more substituents independently selected from —OR⁷ and —OC(O)R⁷. In some embodiments, each R² is independently selected from —CH₂OH and —CH₂OC(O)CH₃.

In some embodiments, each R³ is independently selected from —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R³ is independently selected from halogen, —OR⁷, —SR⁷, —N(R⁷)₂, —C(O)R⁷, —OC(O)R⁷, and —S(O)R⁷. In some embodiments, each R³ is independently selected from —OR⁷, —OC(O)R⁷, —SR⁷, and —N(R⁷)₂. In some embodiments, each R³ is independently selected from —OR⁷ and —OC(O)R⁷. In some embodiments, R³ is independently selected from —OH and —OC(O)CH₃.

In some embodiments, each R⁴ is independently selected from —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R⁴ is independently selected from halogen, —OR⁷, —SR⁷, —N(R⁷)₂, —C(O)R⁷, —OC(O)R⁷, and —S(O)R⁷. In some embodiments, each R⁴ is independently selected from —OR⁷, —OC(O)R⁷, —SR⁷, and —N(R⁷)₂. In some embodiments, each R⁴ is independently selected from —OR⁷ and —OC(O)R⁷. In some embodiments, R⁴ is independently selected from —OH and —OC(O)CH₃.

In some embodiments, each R⁵ is independently selected from—OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂. In some embodiments, each R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷. In some embodiments, each R⁵ is independently selected from —OC(O)R⁷ and —N(R⁷)C(O)R⁷. In some embodiments, each R⁵ is independently selected from —N(H)C(O)CH₃.

In some embodiments, each R⁶ is independently selected from hydrogen, halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and C₁₋₆ alkyl optionally substituted with one or more substituents independently selected from halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷. In some embodiments, each R⁶ is independently selected from hydrogen, halogen, —CN—OR⁷, —SR⁷, —N(R⁷)₂, and C₁₋₆ alkyl optionally substituted with one or more substituents independently selected from halogen, —OR⁷, —SR⁷, and —N(R⁷)₂. In some embodiments, each R⁶ is independently selected from hydrogen, halogen, —CN, —OH, —SH, and —NH₂.

In some embodiments, each R⁷ is independently selected from: hydrogen; C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and C₃₋₁₀ carbocycle, and 3- to 10-membered heterocycle, each of which is optionally substituted with one or more substituents independently selected from halogen, —CN, —OH, —SH, —NO₂, —NH₂, =0, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆ haloalkyl.

In some embodiments, each R⁷ is independently selected from: hydrogen; and C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, 3- to 10-membered heterocycle. In some embodiments, each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl). In some embodiments, each R⁷ is independently selected from hydrogen. In some embodiments, each R⁷ is independently selected from C₁₋₃ alkyl optionally substituted with one or more substituents independently selected from halogen, —CN, —OH, —SH, —NO₂, —NH₂, —O—C₁₋₆alkyl, —S—C₁₋₆ alkyl, —N(C₁₋₆ alkyl)₂, and —NH(C₁₋₆ alkyl).

In some embodiments, a compound of Formula (A) is selected from:

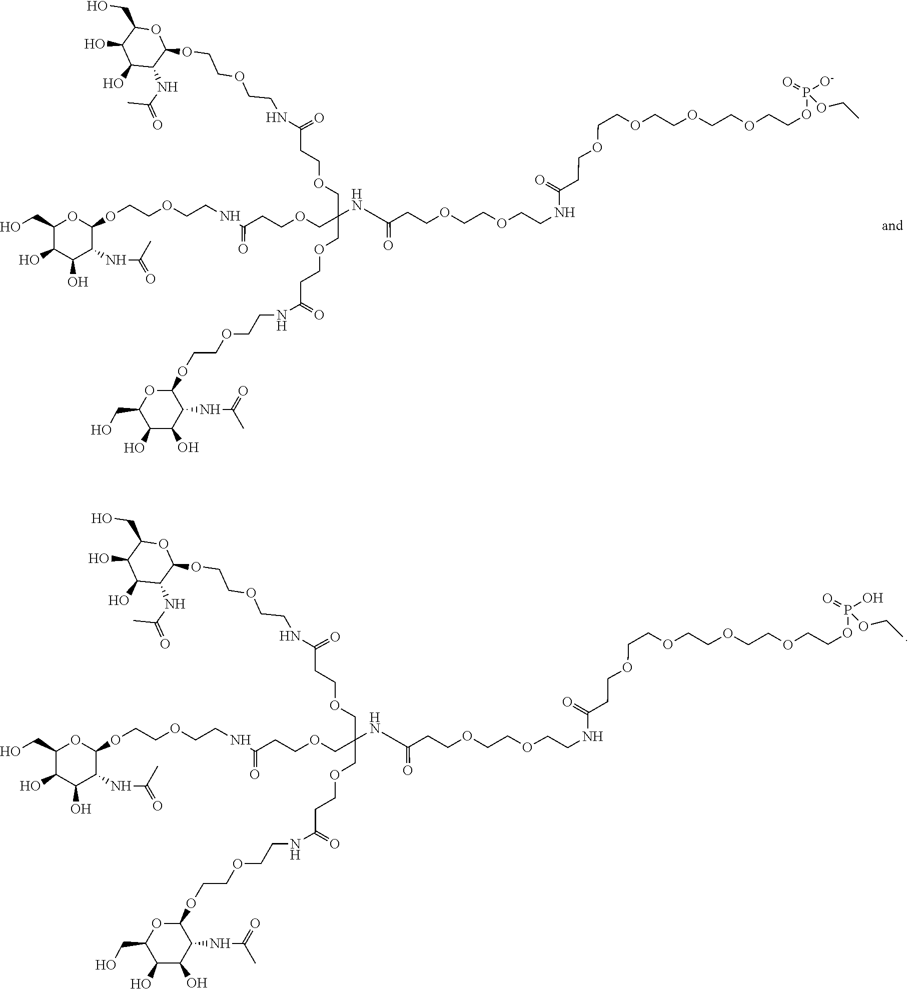 [SMILES: CCOP(=O)([O-])OCCOCCOCCOCCOCCC(=O)NCCOCCOCCC(=O)NC(COCCC(=O)NCCOCCO[C@@H]1O[C@H](CO)[C@H](O)[C@H](O)[C@H]1NC(C)=O)(COCCC(=O)NCCOCCO[C@@H]1O[C@H](CO)[C@H](O)[C@H](O)[C@H]1NC(C)=O)COCCC(=O)NCCOCCO[C@@H]1O[C@H](CO)[C@H](O)[C@H](O)[C@H]1NC(C)=O ; CCOP(=O)(O)OCCOCCOCCOCCOCCC(=O)NCCOCCOCCC(=O)NC(COCCC(=O)NCCOCCO[C@@H]1O[C@H](CO)[C@H](O)[C@H](O)[C@H]1NC(C)=O)(COCCC(=O)NCCOCCO[C@@H]1O[C@H](CO)[C@H](O)[C@H](O)[C@H]1NC(C)=O)COCCC(=O)NCCOCCO[C@@H]1O[C@H](CO)[C@H](O)[C@H](O)[C@H]1NC(C)=O]

Provided herein, in some embodiments, is a compound represented by Formula (B):

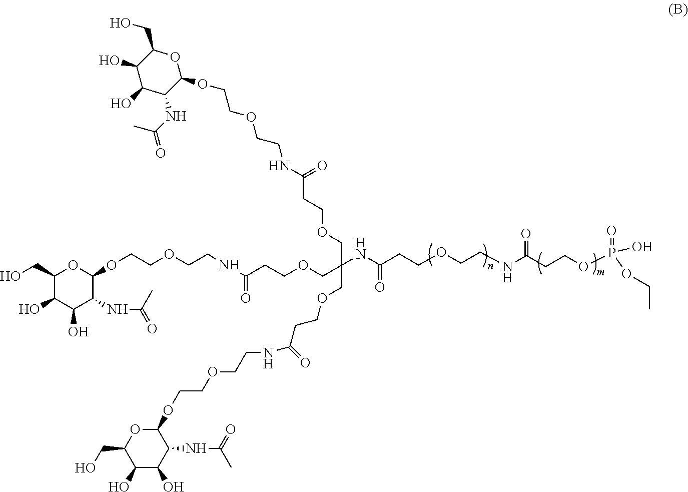

or a salt thereof, wherein
n is selected from any value from 1 to 10; and
m is selected from any value from 1 to 10.

In some embodiments, n is selected from any value from 1 to 10. In some embodiments, n is selected from any value from 1 to 9. In some embodiments, n is selected from any value from 1 to 8. In some embodiments, n is selected from any value from 1 to 7. In some embodiments, n is selected from any value from 1 to 6. In some embodiments, n is selected from any value from 1 to 5. In some embodiments, n is selected from any value from 1 to 4. In some embodiments, n is selected from any value from 2 to 4. In some embodiments, n is selected from any value from 1 to 3. In some embodiments, n is 2 or 3. In some embodiments, n is 3. In some embodiments, n is selected from any value from 1 to 2. In some embodiments, n is 2. In some embodiments, n is 1. In some embodiments, n is 1, 2, 3, 4, 5, 6, 7, 8, 9, or 10.

In some embodiments, m is selected from any value from 1 to 10. In some embodiments, m is selected from any value from 1 to 9. In some embodiments, m is selected from any value from 1 to 8. In some embodiments, m is selected from any value from 1 to 7. In some embodiments, m is selected from any value from 3 to 7. In some embodiments, m is selected from any value from 1 to 6. In some embodiments, m is selected from any value from 2 to 6. In some embodiments, m is selected from any value from 3 to 6. In some embodiments, m is selected from any value from 4 to 6. In some embodiments, m is 6. In some embodiments, m is selected from any value from 1 to 5. In some embodiments, m is selected from any value from 3 to 5. In some embodiments, m is 5. In some embodiments, m is selected from any value from 1 to 4. In some embodiments, m is 4. In some embodiments, m is 3 or 4. In some embodiments, m is selected from any value from 2 to 4. In some embodiments, m is selected from any value from 1 to 3. In some embodiments, m is 3. In some embodiments, m is selected from any value from 1 to 2. In some embodiments, m is 2. In some embodiments, m is 1. In some embodiments, m is 1, 2, 3, 4, 5, 6, 7, 8, 9, or 10.

In some embodiments, the phosphate is deprotonated to form a salt of Formula (B). In some embodiments, the cation is a metal ion. Non limiting examples of metal cations include Na⁺, K⁺, Mg²⁺, and Ca²⁺. In some embodiments, the metal cation comprises Na⁺. In some embodiments, the metal cation comprises K⁺. In some embodiments, the metal cation comprises Mg²⁺. In some embodiments, the metal cation comprises Ca²⁺. In some embodiments, the cation is an organic or inorganic small molecule. In some embodiments, the cation is a positively charged nucleic acid present in the oligonucleotide.

Provided herein, in some embodiments, is a synthesis compound represented by Formula (C):

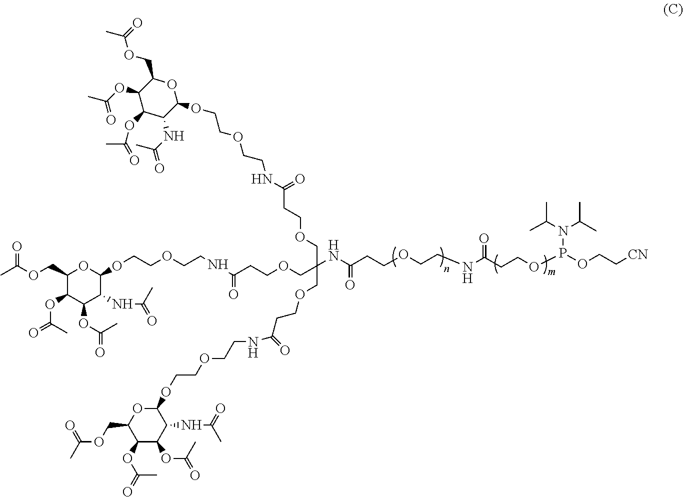

or a salt thereof, wherein
n is selected from any value from 1 to 10; and
m is selected from any value from 1 to 10.

In some embodiments, n is selected from any value from 1 to 10. In some embodiments, n is selected from any value from 1 to 9. In some embodiments, n is selected from any value from 1 to 8. In some embodiments, n is selected from any value from 1 to 7. In some embodiments, n is selected from any value from 1 to 6. In some embodiments, n is selected from any value from 1 to 5. In some embodiments, n is selected from any value from 1 to 4. In some embodiments, n is selected from any value from 2 to 4. In some embodiments, n is selected from any value from 1 to 3. In some embodiments, n is 2 or 3. In some embodiments, n is 3. In some embodiments, n is selected from any value from 1 to 2. In some embodiments, n is 2. In some embodiments, n is 1. In some embodiments, n is 1, 2, 3, 4, 5, 6, 7, 8, 9, or 10.

In some embodiments, m is selected from any value from 1 to 10. In some embodiments, m is selected from any value from 1 to 9. In some embodiments, m is selected from any value from 1 to 8. In some embodiments, m is selected from any value from 1 to 7. In some embodiments, m is selected from any value from 3 to 7. In some embodiments, m is selected from any value from 1 to 6. In some embodiments, m is selected from any value from 2 to 6. In some embodiments, m is selected from any value from 3 to 6. In some embodiments, m is selected from any value from 4 to 6. In some embodiments, m is 6. In some embodiments, m is selected from any value from 1 to 5. In some embodiments, m is selected from any value from 3 to 5. In some embodiments, m is 5. In some embodiments, m is selected from any value from 1 to 4. In some embodiments, m is 4. In some embodiments, m is 3 or 4. In some embodiments, m is selected from any value from 2 to 4. In some embodiments, m is selected from any value from 1 to 3. In some embodiments, m is 3. In some embodiments, m is selected from any value from 1 to 2. In some embodiments, m is 2. In some embodiments, m is 1. In some embodiments, m is 1, 2, 3, 4, 5, 6, 7, 8, 9, or 10.

In some embodiments, a compound of Formula (A), (B), or (C) is an intermediate in the formation of a compound of Formula (I), (II), or (III).

Disclosed herein, in some embodiments are methods of making a compound described herein. In some embodiments, a synthesis compound is used to make a compound of Formula (I).

In some embodiments, a synthesis compound is used to make a compound of Formula (II). In some embodiments, a synthesis compound is used to make a compound of Formula (III). In some embodiments, a synthesis compound is used to make a compound of Formula (A). In some embodiments, a synthesis compound is used to make a compound of Formula (B). In some embodiments, the synthesis compound comprises a phosphoramidite. In some embodiments, the synthesis compound comprises a phosphoramidite version of a compound of Formula (I), (II), (III), (A), or (B). In some embodiments, the synthesis compound comprises a cyano group. The cyano group may comprise a protecting group. In some embodiments, the synthesis compound comprises a cyanoalkyl group. In some embodiments, the synthesis compound comprises a cyanomethylgroup. In some embodiments, the synthesis compound comprises a cyanoethylgroup. In some embodiments, the synthesis compound comprises a synthesis compound of Formula (C). In some embodiments, the synthesis compound comprises:

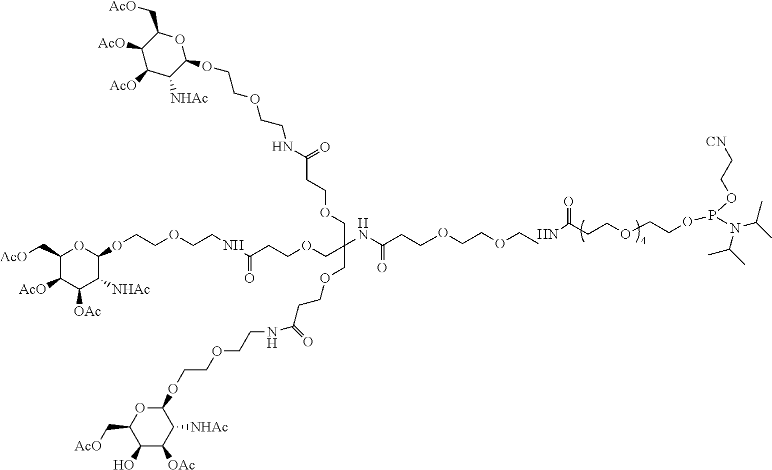

In some embodiments, the synthesis compound comprises

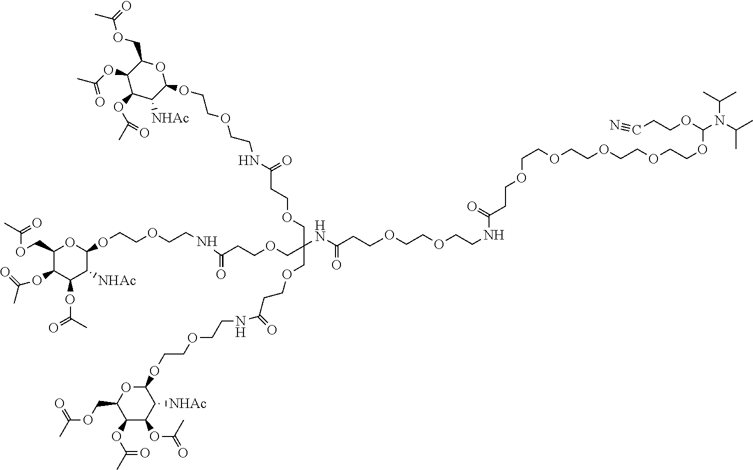 [SMILES: CC(=O)N[C@H]1[C@H](OCCOCCNC(=O)CCOCC(COCCC(=O)NCCOCCO[C@@H]2O[C@H](COC(C)=O)[C@H](OC(C)=O)[C@H](OC(C)=O)[C@H]2NC(C)=O)(COCCC(=O)NCCOCCO[C@@H]2O[C@H](COC(C)=O)[C@H](OC(C)=O)[C@H](OC(C)=O)[C@H]2NC(C)=O)NC(=O)CCOCCOCCNC(=O)CCOCCOCCOCCOCCOC(OCCC#N)N(C(C)C)C(C)C)O[C@H](COC(C)=O)[C@H](OC(C)=O)[C@@H]1OC(C)=O]

1. Analogues

Chemical entities having carbon-carbon double bonds or carbon-nitrogen double bonds may exist in Z- or E-form (or cis- or trans-form). Furthermore, some chemical entities may exist in various tautomeric forms. Unless otherwise specified, compounds described herein are intended to include all Z-, E- and tautomeric forms as well.

A “tautomer” refers to a molecule or moiety wherein a proton shift from one atom of a molecule to another atom of the same molecule is possible. The compounds presented herein, in certain embodiments, exist as tautomers. In circumstances where tautomerization is possible, a chemical equilibrium of the tautomers will exist. The exact ratio of the tautomers depends on several factors, including physical state, temperature, solvent, and pH. Some examples of tautomeric equilibrium include:

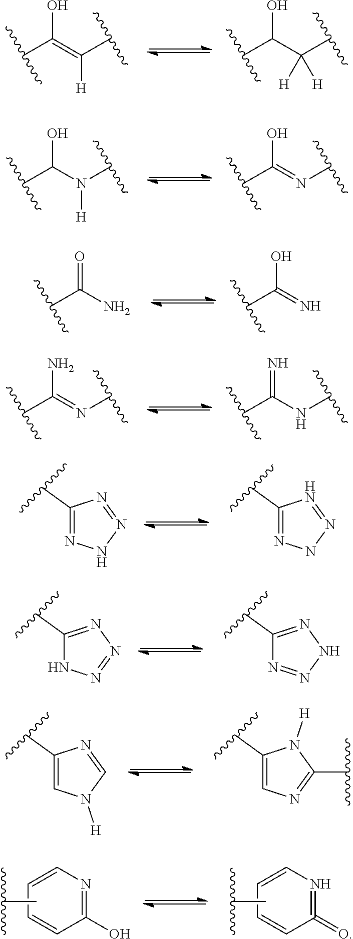

The compounds and moieties disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of d rugs.

Unless otherwise stated, compounds described herein and moieties are intended to include compounds and moieties which differ only in the presence of one or more isotopically enriched atoms. For example, compounds having the present structures except for the replacement of a hydrogen by a deuterium or tritium, or the replacement of a carbon by ¹³C- or 14C-enriched carbon are within the scope of the present disclosure.

The compounds and moieties of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds and moieties of the present invention, whether radioactive or not, are encompassed within the scope of the present invention.

In certain embodiments, the compounds and moieties disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated or solvated forms with pharmaceutically acceptable solvents such as water, ethanol, and the like. The solvated forms of the compounds presented herein are also considered to be disclosed herein.

B. Oligonucleotides

Provided herein, in some embodiments, are compositions or compounds comprising an oligonucleotide. The oligonucleotide may be conjugated to a GalNAc moiety. The oligonucleotide may be directly connected to a linker connected to the GalNAc moiety. The oligonucleotide may be used in a method described herein.

In some embodiments, the oligonucleotide binds to a target oligonucleotide. Examples of target oligonucleotides include a target RNA or a target DNA. In some embodiments, the oligonucleotide binds to a target DNA. In some embodiments, the oligonucleotide binds to a target DNA, and inhibits RNA (e.g. mRNA) expression from the target DNA. In some embodiments, the oligonucleotide binds to a target RNA. The target RNA may include a target mRNA. In some embodiments, the oligonucleotide binds to a target mRNA. In some embodiments, the oligonucleotide inhibits the target mRNA such as by reducing an amount of the target mRNA, causing degradation of the target mRNA, or decreasing or preventing translation of the target mRNA. In some embodiments, the oligonucleotide reduces an amount of a target protein produced from the target mRNA, for example by inhibiting the target mRNA. The oligonucleotide may include a small interfering RNA (siRNA). The oligonucleotide may include an antisense oligonucleotide (ASO).

In some embodiments, the composition comprises an oligonucleotide that binds to a target oligonucleotide and inhibits expression of a target protein encoded by the target oligonucleotide. In some embodiments, the composition comprises an oligonucleotide that binds to a target RNA and inhibits expression of a target protein encoded by the target RNA. In some embodiments, the composition comprises an oligonucleotide that binds to a target mRNA and inhibits expression of a target protein encoded by the target mRNA.

In some embodiments, the composition comprises an oligonucleotide that binds to a target oligonucleotide and inhibits expression of a second oligonucleotide encoded by the target oligonucleotide. In some embodiments, the composition comprises an oligonucleotide that binds to a target DNA and inhibits expression of a target RNA encoded by the target DNA. In some embodiments, the composition comprises an oligonucleotide that binds to a target DNA and inhibits expression of a target mRNA encoded by the target DNA.

Target oligonucleotides may be identified by a variety of ways. In some instances, the target oligonucleotide comprises an mRNA that has expression levels that are associated with incidence of a disorder (e.g. a liver disorder). In some instances, the target oligonucleotide comprises an mRNA that is encoded by a gene that has a particular genotype associated with the disorder. Large-scale human genetic data can improve the success rate of pharmaceutical discovery and development. A Genome Wide Association Study (GWAS) may detect associations between genetic variants and traits in a population sample. A GWAS may enable better understanding of the biology of disease, and provide applicable treatments. A GWAS can utilize genotyping and/or sequencing data, and often involves an evaluation of millions of genetic variants that are relatively evenly distributed across the genome. The most common GWAS design is the case-control study, which involves comparing variant frequencies in cases versus controls. If a variant has a significantly different frequency in cases versus controls, that variant is said to be associated with disease. Association statistics that may be used in a GWAS are p-values, as a measure of statistical significance; odds ratios (OR), as a measure of effect size; or beta coefficients (beta), as a measure of effect size. Researchers often assume an additive genetic model and calculate an allelic odds ratio, which is the increased (or decreased) risk of disease conferred by each additional copy of an allele (compared to carrying no copies of that allele). An additional concept in design and interpretation of GWAS is that of linkage disequilibrium, which is the non-random association of alleles. The presence of linkage disequilibrium can obfuscate which variant is “causal.”

Functional annotation of variants and/or wet lab experimentation can identify the causal genetic variant identified via GWAS, and in many cases may lead to the identification of disease-causing genes. In particular, understanding the functional effect of a causal gen etic variant (for example, loss of protein function, gain of protein function, increase in gene expression, or decrease in gene expression) may allow that variant to be used as a proxy for therapeutic modulation of the target gene, or to gain insight into potential therapeutic efficacy and safety of a therapeutic that modulates that target.

Identification of such gene-disease associations has provided insights into disease biology and may be used to identify novel therapeutic targets for the pharmaceutical industry. In order to translate the therapeutic insights derived from human genetics, disease biology in patients may be exogenously ‘programmed’ into replicating the observation from human genetics. There are several potential options for therapeutic modalities that may be brought to bear in translating therapeutic targets identified via human genetics into novel medicines. These may include well established therapeutic modalities such as small molecules and monoclonal antibodies, maturing modalities such as oligonucleotides, and emerging modalities such as gene therapy and gene editing. The choice of therapeutic modality can depend on several factors including the location of a target (for example, intracellular, extracellular, or secreted), a relevant tissue (for example, liver) and a relevant indication. Such studies may be conducted to identify specific liver disorder-related targets for siRNA or ASO inhibition by a composition or compound described herein.

Some embodiments include a method of making an oligonucleotide or siRNA u sing a method disclosed herein. For example, any aspect of Examples 3, 5, 6, or 13 may be used. Some embodiments include making a GalNAc moiety, or making an oligonucleotide with a GalNAc moiety.

1. siRNAs

In some embodiments, the composition comprises an oligonucleotide that binds to a target oligonucleotide (e.g. mRNA), wherein the oligonucleotide comprises a small interfering RNA (siRNA). In some embodiments, the composition comprises an oligonucleotide that binds to a target oligonucleotide (e.g. mRNA), wherein the oligonucleotide comprises an siRNA comprising a sense strand and an antisense strand. In some embodiments, the sense strand comprises RNA. In some embodiments, the antisense strand comprises RNA.

In some embodiments, the sense strand is 12-30 nucleosides in length. In some embodiments, the sense strand is 14-30 nucleosides in length. In some embodiments, the sense strand is at least about 10, 11, 12, 13, 14, 15, 16, 17, 18, 19, 20, 21, 22, 23, 24, 25, 26, 27, 28, 29, or 30 nucleosides in length. In some embodiments, the sense strand is at least 12 nucleotides in length. In some embodiments, the sense strand is at least 14 nucleotides in length. In some embodiments, the sense strand is at least 16 nucleotides in length. In some embodiments, the sense strand is at least 18 nucleotides in length. In some embodiments, the sense strand is at least 20 nucleotides in length. In some embodiments, the sense strand is at least 22 nucleotides in length. In some embodiments, the sense strand is at least 24 nucleotides in length. In some embodiments, the sense strand is at least 26 nucleotides in length. In some embodiments, the sense strand is at least 28 nucleotides in length. In some embodiments, the sense strand is at least 30 nucleotides in length. In some embodiments, the sense strand is no more than about 10, 11, 12, 13, 14, 15, 16, 17, 18, 19, 20, 21, 22, 23, 24, 25, 26, 27, 28, 29, or 30 nucleosides in length. In some embodiments, the sense strand is no more than 12 nucleotides in length. In some embodiments, the sense strand is no more than 14 nucleotides in length. In some embodiments, the sense strand is no more than 16 nucleotides in length. In some embodiments, the sense strand is no more than 18 nucleotides in length. In some embodiments, the sense strand is no more than 20 nucleotides in length. In some embodiments, the sense strand is no more than 22 nucleotides in length. In some embodiments, the sense strand is no more than 24 nucleotides in length. In some embodiments, the sense strand is no more than 26 nucleotides in length. In some embodiments, the sense strand is no more than 28 nucleotides in length. In some embodiments, the sense strand is no more than 30 nucleotides in length.

In some embodiments, the antisense strand is 12-30 nucleosides in length. In some embodiments, the antisense strand is 14-30 nucleosides in length. In some embodiments, the antisense strand is at least about 10, 11, 12, 13, 14, 15, 16, 17, 18, 19, 20, 21, 22, 23, 24, 25, 26, 27, 28, 29, or 30 nucleosides in length. In some embodiments, the antisense strand is at least 12 nucleotides in length. In some embodiments, the antisense strand is at least 14 nucleotides in length. In some embodiments, the antisense strand is at least 16 nucleotides in length. In some embodiments, the antisense strand is at least 18 nucleotides in length. In some embodiments, the antisense strand is at least 20 nucleotides in length. In some embodiments, the antisense strand is at least 22 nucleotides in length. In some embodiments, the antisense strand is at least 24 nucleotides in length. In some embodiments, the antisense strand is at least 26 nucleotides in length. In some embodiments, the antisense strand is at least 28 nucleotides in length. In some embodiments, the antisense strand is at least 30 nucleotides in length. In some embodiments, the antisense strand is no more than about 10, 11, 12, 13, 14, 15, 16, 17, 18, 19, 20, 21, 22, 23, 24, 25, 26, 27, 28, 29, or 30 nucleosides in length. In some embodiments, the antisense strand is no more than 12 nucleotides in length. In some embodiments, the antisense strand is no more than 14 nucleotides in length. In some embodiments, the antisense strand is no more than 16 nucleotides in length. In some embodiments, the antisense strand is no more than 18 nucleotides in length. In some embodiments, the antisense strand is no more than 20 nucleotides in length. In some embodiments, the antisense strand is no more than 22 nucleotides in length. In some embodiments, the antisense strand is no more than 24 nucleotides in length. In some embodiments, the antisense strand is no more than 26 nucleotides in length. In some embodiments, the antisense strand is no more than 28 nucleotides in length. In some embodiments, the antisense strand is no more than 30 nucleotides in length. In some embodiments, the antisense strand is the same length as the sense strand.

In some embodiments, the composition comprises an oligonucleotide that inhibits the expression of a target oligonucleotide (e.g. mRNA), wherein the oligonucleotide comprises an siRNA comprising a sense strand and an antisense strand, wherein the sense strand is 12-30 nucleosides in length. In some embodiments, the composition comprises a sense strange that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the composition comprises an antisense strand is 12-30 nucleosides in length. In some embodiments, the composition comprises an antisense strange that is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers.

In some embodiments, the composition comprises an oligonucleotide that inhibits the expression of a target oligonucleotide (e.g. mRNA), wherein the oligonucleotide comprises an siRNA comprising a sense strand and an antisense strand, each strand is independently about 14-30 nucleosides in length, and at least one of the sense strand and the antisense strand comprises a nucleoside sequence comprising about 12-30 contiguous nucleosides of a full-length human target mRNA sequence. In some embodiments, at least one of the sense strand and the antisense strand comprise a nucleoside sequence comprising at least about 10, 11, 12, 13, 14, 15, 16, 17, 18, 19, 20, 21, 22, 23, 24, 25, 26, 27, 28, 29, 30, or more contiguous nucleosides of one of the full-length human target mRNA sequence.

In some embodiments, the composition comprises an oligonucleotide that inhibits the expression of a target protein, wherein the oligonucleotide comprises an siRNA comprising a sense strand and an antisense strand, wherein the sense strand and the antisense strand form a double-stranded RNA duplex. In some embodiments, the first base pair of the double-stranded RNA duplex is an AU base pair.

In some embodiments, the sense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the sense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the antisense strand further comprises a 3′ overhang. In some embodiments, the 3′ overhang comprises 1, 2, 3, 4, 5, 6, 7, 8, 9, or 10 nucleosides, or a range of nucleotides defined by any two of the aforementioned numbers. In some embodiments, the 3′ overhang comprises 1, 2, or more nucleosides. In some embodiments, the 3′ overhang comprises 2 nucleosides. In some embodiments, the antisense strand further comprises a 5′ overhang. In some embodiments, the 5′ overhang comprises 1, 2, 3, 4, 5, 6, 7, 8, 9, or 10 nucleosides, or a range of nucleotides defined by any two of the aforementioned numbers. In some embodiments, the 5′ overhang comprises 1, 2, or more nucleosides. In some embodiments, the 5′ overhang comprises 2 nucleosides.

In some embodiments, the composition comprises an oligonucleotide that inhibits the expression of a target protein, wherein the oligonucleotide comprises an siRNA comprising a sense strand and an antisense strand, wherein the siRNA binds with a 19mer in a human target mRNA encoding the target protein. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a human target mRNA.

In some embodiments, the composition comprises an oligonucleotide that inhibits the expression of a target protein, wherein the oligonucleotide comprises an siRNA comprising a sense strand and an antisense strand, wherein the siRNA binds with a 17mer in a non-human primate target mRNA encoding the target protein. In some embodiments, the siRNA binds with a 12mer, a 13mer, a 14mer, a 15mer, a 16mer, a 17mer, a 18mer, a 19mer, a 20mer, a 21mer, a 22mer, a 23mer, a 24mer, or a 25mer in a non-human primate target mRNA.

In some embodiments, the composition comprises an oligonucleotide that inhibits the expression of a target protein, wherein the oligonucleotide comprises an siRNA comprising a sense strand and an antisense strand, wherein the siRNA binds with a 19mer in a human target mRNA encoding the target protein, or a combination thereof. In some embodiments, the siRNA binds with a 12mer, a 13mer, a 14mer, a 15mer, a 16mer, a 17mer, and 18mer, a 19mer, a 20mer, a 21mer, a 22mer, a 23mer, a 24mer, or a 25mer in a human target mRNA.

2. ASOs

In some embodiments, the composition comprises an oligonucleotide that inhibits expression of a target oligonucleotide (e.g. mRNA), wherein the oligonucleotide comprises an antisense oligonucleotide (ASO). In some embodiments, the ASO is 12-30 nucleosides in length. In some embodiments, the ASO is 14-30 nucleosides in length. In some embodiments, the ASO is 10, 11, 12, 13, 14, 15, 16, 17, 18, 19, 20, 21, 22, 23, 24, 25, 26, 27, 28, 29, or 30 nucleosides in length, or a range defined by any of the two aforementioned numbers. In some embodiments, the ASO is 15-25 nucleosides in length. In some embodiments, the ASO is 20 nucleosides in length. In some embodiments, the ASO comprises DNA.

In some embodiments, the ASO is 12-30 nucleosides in length. In some embodiments, the ASO is 14-30 nucleosides in length. In some embodiments, the ASO is at least about 10, 11, 12, 13, 14, 15, 16, 17, 18, 19, 20, 21, 22, 23, 24, 25, 26, 27, 28, 29, or 30 nucleosides in length. In some embodiments, the ASO is at least 12 nucleotides in length. In some embodiments, the ASO is at least 14 nucleotides in length. In some embodiments, the ASO is at least 16 nucleotides in length. In some embodiments, the ASO is at least 18 nucleotides in length. In some embodiments, the ASO is at least 20 nucleotides in length. In some embodiments, the ASO is at least 22 nucleotides in length. In some embodiments, the ASO is at least 24 nucleotides in length. In some embodiments, the ASO is at least 26 nucleotides in length. In some embodiments, the ASO is at least 28 nucleotides in length. In some embodiments, the ASO is at least 30 nucleotides in length. In some embodiments, the ASO is no more than about 10, 11, 12, 13, 14, 15, 16, 17, 18, 19, 20, 21, 22, 23, 24, 25, 26, 27, 28, 29, or 30 nucleosides in length. In some embodiments, the ASO is no more than 12 nucleotides in length. In some embodiments, the ASO is no more than 14 nucleotides in length. In some embodiments, the ASO is no more than 16 nucleotides in length. In some embodiments, the ASO is no more than 18 nucleotides in length. In some embodiments, the ASO is no more than 20 nucleotides in length. In some embodiments, the ASO is no more than 22 nucleotides in length. In some embodiments, the ASO is no more than 24 nucleotides in length. In some embodiments, the ASO is no more than 26 nucleotides in length. In some embodiments, the ASO is no more than 28 nucleotides in length. In some embodiments, the ASO is no more than 30 nucleotides in length.

In some embodiments, the composition comprises an oligonucleotide that inhibits the expression of a target protein, wherein the oligonucleotide comprises an ASO about 12-30 nucleosides in length and comprising a nucleoside sequence complementary to about 12-30 contiguous nucleosides of a full-length human pre-mRNA target sequence encoding the target protein; wherein (i) the oligonucleotide comprises a modification comprising a modified nucleoside and/or a modified internucleoside linkage, and/or (ii) the composition comprises a pharmaceutically acceptable carrier.

In some embodiments, the composition comprises an oligonucleotide that inhibits the expression of a target protein, wherein the oligonucleotide comprises an ASO about 12-30 nucleosides in length and comprising a nucleoside sequence complementary to about 12-30 contiguous nucleosides of a full-length human target mRNA sequence encoding the target protein; wherein (i) the oligonucleotide comprises a modification comprising a modified nucleoside and/or a modified internucleoside linkage, and/or (ii) the composition comprises a pharmaceutically acceptable carrier.

1. Oligonucleotide Modification Patterns

In some embodiments, the composition comprises an oligonucleotide that binds to a target oligonucleotide, wherein the oligonucleotide comprises a modification comprising a modified nucleoside and/or a modified internucleoside linkage, and/or (ii) the composition comprises a pharmaceutically acceptable carrier. In some embodiments, the oligonucleotide comprises a modification comprising a modified nucleoside and/or a modified internucleoside linkage. In some embodiments, the oligonucleotide comprises a modified internucleoside linkage. In some embodiments, the modified internucleoside linkage comprises alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof. In some embodiments, the modified internucleoside linkage comprises one or more phosphorothioate linkages. Benefits of the modified internucleoside linkage may include decreased toxicity or improved pharmacokinetics.

In some embodiments, the composition comprises an oligonucleotide that binds to a target oligonucleotide, wherein the oligonucleotide comprises a modified internucleoside linkage, wherein the oligonucleotide comprises 1, 2, 3, 4, 5, 6, 7, 8, 9, 10, 11, 12, 13, 14, 15, 16, 17, 18, 19, or 20 modified internucleoside linkages, or a range of modified internucleoside linkages defined by any two of the aforementioned numbers. In some embodiments, the oligonucleotide comprises no more than 18 modified internucleoside linkages. In some embodiments, the oligonucleotide comprises no more than 20 modified internucleoside linkages. In some embodiments, the oligonucleotide comprises 2 or more modified internucleoside linkages, 3 or more modified internucleoside linkages, 4 or more modified internucleoside linkages, 5 or more modified internucleoside linkages, 6 or more modified internucleoside linkages, 7 or more modified internucleoside linkages, 8 or more modified internucleoside linkages, 9 or more modified internucleoside linkages, 10 or more modified internucleoside linkages, 11 or more modified internucleoside linkages, 12 or more modified internucleoside linkages, 13 or more modified internucleoside linkages, 14 or more modified internucleoside linkages, 15 or more modified internucleoside linkages, 16 or more modified internucleoside linkages, 17 or more modified internucleoside linkages, 18 or more modified internucleoside linkages, 19 or more modified internucleoside linkages, or 20 or more modified internucleoside linkages.

In some embodiments, the composition comprises an oligonucleotide that binds to a target oligonucleotide, wherein the oligonucleotide comprises the modified nucleoside. In some embodiments, the modified nucleoside comprises a locked nucleic acid (LNA), hexitol nucleic acid (HLA), cyclohexene nucleic acid (CeNA), 2′-methoxyethyl, 2′-O-alkyl, 2′-O-allyl, 2′-fluoro, or 2′-deoxy, or a combination thereof. In some embodiments, the modified nucleoside comprises a LNA. In some embodiments, the modified nucleoside comprises a 2′,4′ constrained ethyl nucleic acid. In some embodiments, the modified nucleoside comprises HLA. In some embodiments, the modified nucleoside comprises CeNA. In some embodiments, the modified nucleoside comprises a 2′-methoxyethyl group. In some embodiments, the modified nucleoside comprises a 2′-O-alkyl group. In some embodiments, the modified nucleoside comprises a 2′-O-allyl group. In some embodiments, the modified nucleoside comprises a 2′-fluoro group. In some embodiments, the modified nucleoside comprises a 2′-deoxy group. In some embodiments, the modified nucleoside comprises a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, or 2′-ara-F, or a combination thereof. In some embodiments, the modified nucleoside comprises a 2′-O-methyl nucleoside. In some embodiments, the modified nucleoside comprises a 2′-deoxyfluoro nucleoside. In some embodiments, the modified nucleoside comprises a 2′-O-NMA nucleoside. In some embodiments, the modified nucleoside comprises a 2′-O-DMAEOE nucleoside. In some embodiments, the modified nucleoside comprises a 2′-O-aminopropyl (2′-O-AP) nucleoside. In some embodiments, the modified nucleoside comprises 2′-ara-F. In some embodiments, the modified nucleoside comprises one or more 2′fluoro modified nucleosides. In some embodiments, the modified nucleoside comprises a 2′ O-alkyl modified nucleoside. Benefits of the modified nucleoside may include decreased toxicity or improved pharmacokinetics.

In some embodiments, the oligonucleotide comprises 1, 2, 3, 4, 5, 6, 7, 8, 9, 10, 11, 12, 13, 14, 15, 16, 17, 18, 19, 20, or 21 modified nucleosides, or a range of nucleosides defined by any two of the aforementioned numbers. In some embodiments, the oligonucleotide comprises no more than 19 modified nucleosides. In some embodiments, the oligonucleotide comprises no more than 21 modified nucleosides. In some embodiments, the oligonucleotide comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, or 21 or more modified nucleosides.

In some embodiments, the composition comprises an oligonucleotide that binds to a target oligonucleotide, wherein the oligonucleotide comprises a lipid attached at a 3′ or 5′ terminus of the oligonucleotide. In some embodiments, the lipid comprises cholesterol, myristoyl, palmitoyl, stearoyl, lithocholoyl, docosanoyl, docosahexaenoyl, myristyl, palmityl stearyl, or α-tocopherol, or a combination thereof.

In some embodiments, the composition comprises an oligonucleotide that binds to a target oligonucleotide, wherein the oligonucleotide comprises an N-acetylgalactosamine (GalNAc) moiety for hepatocyte targeting. In some embodiments, the composition comprises GalNAc. In some embodiments, the composition comprises a GalNAc derivative.

2. siRNA Modification Patterns

In some embodiments, the sense strand comprises 1, 2, 3, 4, 5, 6, 7, 8, 9, 10, 11, 12, 13, 14, 15, 16, 17, 18, 19, 20, 21, 22, 23, 24, 25, 26, 27, 28, or 29 modified internucleoside linkages, or a range of modified internucleoside linkages defined by any two of the aforementioned integers. In some embodiments, the sense strand comprises 1-11 modified internucleoside linkages. In some embodiments, the sense strand comprises 2-6 modified internucleoside linkages. In some embodiments, the sense strand comprises 5 modified internucleoside linkages. In some embodiments, the sense strand comprises 4 modified internucleoside linkages.

In some embodiments, the antisense strand comprises 1, 2, 3, 4, 5, 6, 7, 8, 9, 10, 11, 12, 13, 14, 15, 16, 17, 18, 19, 20, 21, 22, 23, 24, 25, 26, 27, 28, or 29 modified internucleoside linkages, or a range of modified internucleoside linkages defined by any two of the aforementioned integers. In some embodiments, the antisense strand comprises 1-11 modified internucleoside linkages. In some embodiments, the antisense strand comprises 2-6 modified internucleoside linkages. In some embodiments, the antisense strand comprises 5 modified internucleoside linkages. In some embodiments, the antisense strand comprises 4 modified internucleoside linkages.

In some embodiments, the sense strand comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 modified nucleosides, or a range of modified nucleosides defined by any two of the aforementioned integers. In some embodiments, the sense strand comprises 12-19 modified nucleosides. In some embodiments, the sense strand comprises 12-21 modified nucleosides. In some embodiments, the sense strand comprises 19 modified nucleosides. In some embodiments, the sense strand comprises 21 modified nucleosides.

In some embodiments, the antisense strand comprises 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 modified nucleosides, or a range of modified nucleosides defined by any two of the aforementioned integers. In some embodiments, the antisense strand comprises 12-19 modified nucleosides. In some embodiments, the antisense strand comprises 12-21 modified nucleosides. In some embodiments, the antisense strand comprises 19 modified nucleosides. In some embodiments, the antisense strand comprises 21 modified nucleosides.

In some embodiments, the sense strand or the antisense strand further comprises at least 2 additional nucleosides attached to a 3′ terminus of the sense strand or the antisense strand. In some embodiments, the sense strand or the antisense strand comprises 2 additional nucleosides attached to a 3′ terminus of the sense strand or the antisense strand. As part of the sense strand, the additional nucleosides may or may not be complementary to a target mRNA. The additional nucleosides of the antisense strand may include a uracil. The 2 additional nucleosides of the antisense strand may both include uracil.

In some embodiments, the sense strand or the sense strand further comprises at least 2 additional nucleosides attached to a 3′ terminus of the sense strand or the sense strand. In some embodiments, the sense strand or the sense strand comprises 2 additional nucleosides attached to a 3′ terminus of the sense strand or the sense strand. The additional nucleosides of the sense strand may include a uracil. The 2 additional nucleosides of the sense strand may both include uracil.

In some embodiments, the composition comprises an oligonucleotide that binds to a target oligonucleotide, wherein the oligonucleotide comprises an siRNA comprising a sense strand and an antisense strand, wherein the sense strand comprises a modification pattern. In some embodiments, the sense strand comprises modification pattern 1 S: 5′-NfsnsNfnNfnNfNfNfnNfnNfnNfnNfnNfsnsn-3′ (SEQ ID NO: 1), wherein “Nf” is a 2′ fluoro-modified nucleoside, “n” is a 2′ O-methyl modified nucleoside, and “s” is a phosphorothioate linkage. In some embodiments, the sense strand comprises modification pattern 1 S #2: 5′-NfnNfnNfnNfNfNfnNfnNfnNfnNfnNfsnsn-3′ (SEQ ID NO: 2), wherein “Nf” is a 2′ fluoro-modified nucleoside, “n” is a 2′ O-methyl modified nucleoside, and “s” is a phosphorothioate linkage. In some embodiments, the sense strand comprises modification pattern 2S: 5′-nsnsnnNfnNfNfNfnnnnnnnnnnsnsn-3′ (SEQ ID NO: 3), wherein “Nf” is a 2′ fluoro-modified nucleoside, “n” is a 2′ O-methyl modified nucleoside, and “s” is a phosphorothioate linkage. In some embodiments, the sense strand comprises modification pattern 2S #2: 5′-nnnnNfnNfNfNfnnnnnnnnnnsnsn-3′ (SEQ ID NO: 4), wherein “Nf” is a 2′ fluoro-modified nucleoside, “n” is a 2′ O-methyl modified nucleoside, and “s” is a phosphorothioate linkage. In some embodiments, the sense strand comprises modification pattern 3 S: 5′-nsnsnnNfnNfnNfnnnnnnnnnnsnsn-3′ (SEQ ID NO: 5), wherein “Nf” is a 2′ fluoro-modified nucleoside, “n” is a 2′ O-methyl modified nucleoside, and “s” is a phosphorothioate linkage. In some embodiments, the sense strand comprises modification pattern 3 S #2: 5′-nnnnNfnNfnNfnnnnnnnnnnsnsn-3′ (SEQ ID NO: 6), wherein “Nf” is a 2′ fluoro-modified nucleoside, “n” is a 2′ O-methyl modified nucleoside, and “s” is a phosphorothioate linkage. In some embodiments, the sense strand comprises modification pattern 4S: 5′-NfsnsNfnNfnNfNfNfnNfnNfnNfnNfnNfsnsnN-3′ (SEQ ID NO: 7), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 4S #2: 5′-NfnNfnNfnNfNfNfnNfnNfnNfnNfnNfsnsnN-3′ (SEQ ID NO: 8), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S: 5′-nsnsnnNfnNfNfNfnnnnnnnnnnsnsnN-3′ (SEQ ID NO: 9), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 5S #2: 5′-nnnnNfnNfNfNfnnnnnnnnnnsnsnN-3′ (SEQ ID NO: 10), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 6S: 5′-NfsnsNfnNfnNfnNfnNfnNfnNfnNfnNfsnsn-3′ (SEQ ID NO: 11), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises modification pattern 6S #2: 5′-NfnNfnNfnNfnNfnNfnNfnNfnNfnNfsnsn-3′ (SEQ ID NO: 12), wherein “Nf” is a 2′ fluoro-modified nucleoside, “n” is a 2′ O-methyl modified nucleoside, “s” is a phosphorothioate linkage, and N comprises one or more nucleosides. In some embodiments, the sense strand comprises any one of modification patterns 1S, 2S, 3S, 4S, 5S, or 6S. In some embodiments, the sense strand comprises any one of modification patterns 1S #2, 2S #2, 3 S #2, 4S #2, 5S #2, or 6S #2. In some embodiments, the sense strand comprises any one of modification patterns 1S, 3S, 4S, or 6S. In some embodiments, the sense strand comprises any one of modification patterns 1S #2, 3 S #2, 4 S #2, or 6 S #2. Any one of modification patterns 1S-6S may include a GalNAc ligand attached to the 3′ end. Any one of modification patterns 1S-6S may include a GalNAc ligand attached to the 5′ end. Any one of modification patterns 1S-6S #2 may include a GalNAc ligand attached to the 3′ end. Any one of modification patterns 1 S-6S #2 may include a GalNAc ligand attached to the 5′ end.

In some embodiments, the composition comprises an oligonucleotide that binds to a target oligonucleotide, wherein the oligonucleotide comprises an siRNA comprising a sense strand and an antisense strand, wherein the antisense strand comprises a modification pattern. In some embodiments, the antisense strand comprises modification pattern 1AS: 5′-nsNfsnNfnNfnNfnNfnnnNfnNfnNfnsnsn-3′ (SEQ ID NO: 13), wherein “Nf” is a 2′ fluoro-modified nucleoside, “n” is a 2′ O-methyl modified nucleoside, and “s” is a phosphorothioate linkage. In some embodiments, the antisense strand comprises modification pattern 2AS: 5′-nsNfsnnnNfnNfNfnnnnNfnNfnnnsnsn-3′ (SEQ ID NO: 14), wherein “Nf” is a 2′ fluoro-modified nucleoside, “n” is a 2′ O-methyl modified nucleoside, and “s” is a phosphorothioate linkage. In some embodiments, the antisense strand comprises modification pattern 3AS: 5′-nsNfsnnnNfnnnnnnnNfnNfnnnsnsn-3′ (SEQ ID NO: 15), wherein “Nf” is a 2′ fluoro-modified nucleoside, “n” is a 2′ O-methyl modified nucleoside, and “s” is a phosphorothioate linkage. In some embodiments, the antisense strand comprises modification pattern 4AS: 5′-nsNfsnNfnNfnnnnnnnNfnNfnnnsnsn-3′ (SEQ ID NO: 16), wherein “Nf” is a 2′ fluoro-modified nucleoside, “n” is a 2′ O-methyl modified nucleoside, and “s” is a phosphorothioate linkage. In some embodiments, the antisense strand comprises modification pattern 5AS: 5′-nsNfsnnnNfnNfnnnnnNfnNfnnnsnsn-3′ (SEQ ID NO: 17), wherein “Nf” is a 2′ fluoro-modified nucleoside, “n” is a 2′ O-methyl modified nucleoside, and “s” is a phosphorothioate linkage. In some embodiments, the antisense strand comprises modification pattern 6AS: 5′-nsNfsnNfnNfnNfnNfnNfnNfnNfnNfnsnsn-3′ (SEQ ID NO: 18), wherein “Nf” is a 2′ fluoro-modified nucleoside, “n” is a 2′ O-methyl modified nucleoside, and “s” is a phosphorothioate linkage. In some embodiments, the antisense strand comprises modification pattern 7AS: 5′-nsNfsnNfnNfnNfNfnnnnNfnNfnnnsnsn-3′ (SEQ ID NO: 19), wherein “Nf” is a 2′ fluoro-modified nucleoside, “n” is a 2′ O-methyl modified nucleoside, and “s” is a phosphorothioate linkage. In some embodiments, the antisense strand comprises modification pattern 8AS: 5′-nsNfsnnnnnnnnnnnNfnnnnnsnsn-3′ (SEQ ID NO: 20), wherein “Nf” is a 2′ fluoro-modified nucleoside, “n” is a 2′ O-methyl modified nucleoside, and “s” is a phosphorothioate linkage. In some embodiments, the antisense strand comprises modification pattern 9AS: 5′-nsNfsnnnNfnnnnnnnNfnNfnNfnsnsn-3′ (SEQ ID NO: 21), wherein “Nf” is a 2′ fluoro-modified nucleoside, “n” is a 2′ O-methyl modified nucleoside, and “s” is a phosphorothioate linkage. Any one of modification patterns 1AS-9AS may include a GalNAc ligand attached to the 3′ end. Any one of modification patterns 1AS-9AS may include a GalNAc ligand attached to the 5′ end.

The modifications in any of the modification patterns may be optional. For example, 1, 2, 3, or more phosphorothioate linkages in any of modification patterns 1S-6S, 1 S #2-6S #2, or 1AS-9AS may be replaced with an unmodified linkage, or with a different modified linkage. In some cases, 1, 2, 3, or more modified nucleosides in any of modification patterns 1S-6S or 1AS-9AS may be replaced with an unmodified nucleoside, or with a different modified nucleoside.

In some embodiments, the composition comprises an oligonucleotide that inhibits the expression of a target mRNA wherein the oligonucleotide comprises an siRNA comprising a sense strand and an antisense strand, wherein the sense strand comprises modification pattern 1S and the antisense strand comprises modification pattern 1AS, 2AS, 3AS, 4AS, 5AS, 6AS, 7AS, 8AS, or 9AS. In some embodiments, the sense strand comprises modification pattern 2S and the antisense strand comprises modification pattern 1AS, 2AS, 3AS, 4AS, 5AS, 6AS, 7AS, 8AS, or 9AS. In some embodiments, the sense strand comprises modification pattern 3 S and the antisense strand comprises modification pattern 1AS, 2AS, 3AS, 4AS, 5AS, 6AS, 7AS, 8AS, or 9AS. In some embodiments, the sense strand comprises modification pattern 4S and the antisense strand comprises modification pattern 1AS, 2AS, 3AS, 4AS, 5AS, 6AS, 7AS, 8AS, or 9AS. In some embodiments, the sense strand comprises modification pattern 5S and the antisense strand comprises modification pattern 1AS, 2AS, 3AS, 4AS, 5AS, 6AS, 7AS, 8AS, or 9AS. In some embodiments, the sense strand comprises modification pattern 6S and the antisense strand comprises modification pattern 1AS, 2AS, 3AS, 4AS, 5AS, 6AS, 7AS, 8AS, or 9AS. In some embodiments, the sense strand comprises modification pattern 1AS, 2AS, 3AS, 4AS, 5AS, 6AS, 7AS, 8AS, or 9AS. In some embodiments, the sense strand comprises modification pattern 1 S #2 and the antisense strand comprises modification pattern 1AS, 2AS, 3AS, 4AS, 5AS, 6AS, 7AS, 8AS, or 9AS. In some embodiments, the sense strand comprises modification pattern 2S #2 and the antisense strand comprises modification pattern 1AS, 2AS, 3AS, 4AS, 5AS, 6AS, 7AS, 8AS, or 9AS. In some embodiments, the sense strand comprises modification pattern 3 S #2 and the antisense strand comprises modification pattern 1AS, 2AS, 3AS, 4AS, 5AS, 6AS, 7AS, 8AS, or 9AS. In some embodiments, the sense strand comprises modification pattern 4S #2 and the antisense strand comprises modification pattern 1AS, 2AS, 3AS, 4AS, 5AS, 6AS, 7AS, 8AS, or 9AS. In some embodiments, the sense strand comprises modification pattern 5S #2 and the antisense strand comprises modification pattern 1AS, 2AS, 3AS, 4AS, 5AS, 6AS, 7AS, 8AS, or 9AS. In some embodiments, the sense strand comprises modification pattern 6S #2 and the antisense strand comprises modification pattern 1AS, 2AS, 3AS, 4AS, 5AS, 6AS, 7AS, 8AS, or 9AS. In some embodiments, the antisense strand comprises modification pattern 1AS, 2AS, 3AS, 4AS, 5AS, 6AS, 7AS, 8AS, or 9AS. In some embodiments, the sense strand or the antisense strand comprises modification pattern ASO1.

Any combination of sense and antisense modification patterns may be used. In some embodiments, the sense strand comprises modification pattern 1S, and the antisense strand comprises modification pattern 1AS. In some embodiments, the sense strand comprises modification pattern 2S, and the antisense strand comprises modification pattern 2AS. In some embodiments, the sense strand comprises modification pattern 2S, and the antisense strand comprises modification pattern 3AS. In some embodiments, the sense strand comprises modification pattern 3 S, and the antisense strand comprises modification pattern 1AS. In some embodiments, the sense strand comprises modification pattern 3 S, and the antisense strand comprises modification pattern 4AS. In some embodiments, the sense strand comprises modification pattern 3 S, and the antisense strand comprises modification pattern 5AS. In some embodiments, the sense strand comprises modification pattern 3 S, and the antisense strand comprises modification pattern 6AS. In some embodiments, the sense strand comprises modification pattern 3 S, and the antisense strand comprises modification pattern 7AS. In some embodiments, the sense strand comprises modification pattern 3 S, and the antisense strand comprises modification pattern 8AS. In some embodiments, the sense strand comprises modification pattern 6S, and the antisense strand comprises modification pattern 1AS. In some embodiments, the sense strand comprises modification pattern 6S, and the antisense strand comprises modification pattern 4AS. In some embodiments, the sense strand comprises modification pattern 6S, and the antisense strand comprises modification pattern 5AS. In some embodiments, the sense strand comprises modification pattern 6S, and the antisense strand comprises modification pattern 6AS. In some embodiments, the sense strand comprises modification pattern 6S, and the antisense strand comprises modification pattern 7AS. In some embodiments, the sense strand comprises modification pattern 6S, and the antisense strand comprises modification pattern 8AS. In some embodiments, the sense strand comprises modification pattern 1S #2, and the antisense strand comprises modification pattern 1AS. In some embodiments, the sense strand comprises modification pattern 2S #2, and the antisense strand comprises modification pattern 2AS. In some embodiments, the sense strand comprises modification pattern 2S #2, and the antisense strand comprises modification pattern 3AS. In some embodiments, the sense strand comprises modification pattern 3 S #2, and the antisense strand comprises modification pattern 1AS. In some embodiments, the sense strand comprises modification pattern 3 S #2, and the antisense strand comprises modification pattern 4AS. In some embodiments, the sense strand comprises modification pattern 3 S #2, and the antisense strand comprises modification pattern 5AS. In some embodiments, the sense strand comprises modification pattern 3 S #2, and the antisense strand comprises modification pattern 6AS. In some embodiments, the sense strand comprises modification pattern 3 S #2, and the antisense strand comprises modification pattern 7AS. In some embodiments, the sense strand comprises modification pattern 3 S #2, and the antisense strand comprises modification pattern 8AS. In some embodiments, the sense strand comprises modification pattern 6S #2, and the antisense strand comprises modification pattern 1AS. In some embodiments, the sense strand comprises modification pattern 6S #2, and the antisense strand comprises modification pattern 4AS. In some embodiments, the sense strand comprises modification pattern 6S #2, and the antisense strand comprises modification pattern 5AS. In some embodiments, the sense strand comprises modification pattern 6S #2, and the antisense strand comprises modification pattern 6AS. In some embodiments, the sense strand comprises modification pattern 6S #2, and the antisense strand comprises modification pattern 7AS. In some embodiments, the sense strand comprises modification pattern 6S #2, and the antisense strand comprises modification pattern 8AS. 3. ASO modification patterns

In some embodiments, the composition comprises an oligonucleotide that binds to a target oligonucleotide, wherein the oligonucleotide comprises an antisense oligonucleotide (ASO). In some embodiments, the ASO comprises modification pattern ASO1: 5′-nsnsnsnsnsdNsdNsdNsdNsdNsdNsdNsdNsdNsdNsnsnsnsnsn-3′ (SEQ ID NO: 22), wherein “dN” is any deoxynucleotide, “n” is a 2′O-methyl or 2′O-methoxyethyl-modified nucleoside, and “s” is a phosphorothioate linkage. In some embodiments, the ASO comprises modification pattern 1S, 2S, 3S, 4S, 5S, 6S, 1AS, 2AS, 3AS, 4AS, 5AS, 6AS, 7AS, 8AS, or 9AS.

In some embodiments, the ASO is conjugated to a GalNAc moiety. The GalNAc moiety may be conjugated to the ASO at a 5′ terminus or the 3′ terminus. In some embodiments, the GalNAc moiety is conjugated to the 5′ terminus of the ASO. In some embodiments, the GalNAc moiety is conjugated to the 3′ terminus of the ASO.

C. Formulations

In some embodiments, the composition is a pharmaceutical composition. In some embodiments, the composition is sterile. In some embodiments, the composition further comprises a pharmaceutically acceptable carrier. The formulation may include a compound such as a GalNAc moiety, and an oligonucleotide conjugated to a GalNAc moiety described herein.

In some embodiments, the pharmaceutically acceptable carrier comprises water.

In some embodiments, the pharmaceutically acceptable carrier comprises a buffer. In some embodiments, the pharmaceutically acceptable carrier comprises a saline solution. In some embodiments, the pharmaceutically acceptable carrier comprises water, a buffer, or a saline solution. In some embodiments, the composition comprises a liposome. In some embodiments, the pharmaceutically acceptable carrier comprises liposomes, lipids, nanoparticles, proteins, protein-antibody complexes, peptides, cellulose, nanogel, or a combination thereof.

### II. METHODS AND USES

Disclosed herein, in some embodiments, are methods of administering a composition described herein to a subject. Some embodiments relate to use a composition described herein, such as administering the compositionto a subject.

Some embodiments relate to a method of treating a disorder in a subject in need thereof. Some embodiments relate to use of a composition described herein in the m ethod of treatment. Some embodiments include administering a composition described herein to a subject with the disorder. In some embodiments, the administration treats the disorder in the subject. In some embodiments, the composition treats the disorder in the subject.

In some embodiments, the treatment comprises prevention, inhibition, or reversion of the disorder in the subject. Some embodiments relate to use of a composition described herein in the method of preventing, inhibiting, or reversing the disorder. Some embodiments relate to a method of preventing, inhibiting, or reversing a disorder a disorder in a subject in need thereof. Some embodiments include administering a composition described herein to a subject with the disorder. In some embodiments, the administration prevents, inhibits, or reverses the disorder in the subject. In some embodiments, the composition prevents, inhibits, or reverses the disorder in the subject.

Some embodiments relate to a method of preventing a disorder a disorder in a subject in need thereof. Some embodiments relate to use of a composition described herein in the method of preventing the disorder. Some embodiments include administering a composition described herein to a subject with the disorder. In some embodiments, the administration prevents the disorder in the subject. In some embodiments, the composition prevents the disorder in the subject.

Some embodiments relate to a method of inhibiting a disorder a disorder in a subject in need thereof. Some embodiments relate to use of a composition described herein in the method of inhibiting the disorder. Some embodiments include administering a composition described herein to a subjectwith the disorder. In some embodiments, the administration inhibits the disorder in the subject. In some embodiments, the composition inhibits the disorder in the subject.

Some embodiments relate to a method of reversing a disorder a disorder in a subject in need thereof. Some embodiments relate to use of a composition described herein in the method of reversing the disorder. Some embodiments include administering a composition described herein to a subject with the disorder. In some embodiments, the administration reverses the disorder in the subject. In some embodiments, the composition reverses the disorder in the subject.

A. Disorders

Some embodiments of the methods described herein include treating a disorder in a subject in need thereof. In some embodiments, the disorder is a liver disorder. Non-limiting examples of liver disorders include liver inflammation, liver cancer, liver fibrosis, cholestasis, a gall bladder disease, a biliary tree disease, alcoholic liver disease, non-alcoholic steatohepatitis, a liver infection, or an inherited liver disorder. In some embodiments, the liver disorder comprises liver inflammation. In some embodiments, the liver disorder comprises liver cancer. In some embodiments, the liver disorder comprises liver fibrosis. In some embodiments, the liver disorder comprises cholestasis. In some embodiments, the liver disorder comprises a gall bladder disease. In some embodiments, the liver disorder comprises a biliary tree disease. In some embodiments, the liver disorder comprises alcoholic liver disease. In some embodiments, the liver disorder comprises non-alcoholic steatohepatitis.

In some embodiments, the liver disorder comprises a liver infection. In some embodiments, the liver infection comprises hepatitis A. In some embodiments, the liver infection comprises hepatitis B. In some embodiments, the liver infection comprises hepatitis C.

In some embodiments, the liver disorder comprises an inherited liver disorder. In some embodiments, the inherited liver disorder comprises hemochromatosis. In some embodiments, the inherited liver disorder comprises Wilson disease.

B. Subjects

Some embodiments of the methods described herein include treatment of a subject. Non-limiting examples of subjects include vertebrates, animals, mammals, dogs, cats, cattle, rodents, mice, rats, primates, monkeys, and humans. In some embodiments, the subject is a vertebrate. In some embodiments, the subject is an animal. In some embodiments, the subject is a mammal. In some embodiments, the subject is a dog. In some embodiments, the subject is a cat.

In some embodiments, the subject is a cattle. In some embodiments, the subject is a mouse. In some embodiments, the subject is a rat. In some embodiments, the subject is a primate. In some embodiments, the subject is a monkey. In some embodiments, the subject is an animal, a mammal, a dog, a cat, cattle, a rodent, a mouse, a rat, a primate, or a monkey. In some embodiments, the subject is a human.

In some embodiments, the subject is male. In some embodiments, the subject is female.

In some embodiments, the subject has a body mass index (BMI) of 25, 26, 27, 28, 29, 30, 31, 32, 33, 34, 35, 36, 37, 38, 39, 40, 41, 42, 43, 44, 45, 46, 47, 48, 49, 50, or more, or a range defined by any two of the aforementioned integers. In some embodiments, the subject is overweight. In some embodiments, the subject has a BMI of 25 or more. In some embodiments, the subject has a BMI of 25-29. In some embodiments, the subject is obese. In some embodiments, the subject has a BMI of 30 or more. In some embodiments, the subject has a BMI of 30-39. In some embodiments, the subject has a BMI of 40-50. In some embodiments, the subject has a BMI of 25-50.

In some embodiments, the subject is ≥90 years of age. In some embodiments, the subject is ≥85 years of age. In some embodiments, the subject is ≥80 years of age. In some embodiments, the subject is 270 years of age. In some embodiments, the subject is 60 years of age. In some embodiments, the subject is ≥50 years of age. In some embodiments, the subject is ≥40 years of age. In some embodiments, the subject is ≥30 years of age. In some embodiments, the subject is ≥20 years of age. In some embodiments, the subject is ≥10 years of age. In some embodiments, the subject is ≥1 years of age. In some embodiments, the subject is 20 years of age.

In some embodiments, the subject is ≤100 years of age. In some embodiments, the subject is ≤90 years of age. In some embodiments, the subject is 85 years of age. In some embodiments, the subject is ≤80 years of age. In some embodiments, the subject is 70 years of age. In some embodiments, the subject is 60 years of age. In some embodiments, the subject is ≤50 years of age. In some embodiments, the subject is 40 years of age. In some embodiments, the subject is 30 years of age. In some embodiments, the subject is 20 years of age. In some embodiments, the subject is 10 years of age. In some embodiments, the subject is ≤1 years of age.

In some embodiments, the subject is between 0 and 100 years of age. In some embodiments, the subject is between 20 and 90 years of age. In some embodiments, the subject is between 30 and 80 years of age. In some embodiments, the subject is between 40 and 75 years of age. In some embodiments, the subject is between 50 and 70 years of age. In some embodiments, the subject is between 40 and 85 years of age.

C. Baseline Measurements

Some embodiments of the methods described herein include obtaining a baseline measurement from a subject. For example, in some embodiments, a baseline measurement is obtained from the subject prior to treating the subject. Non-limiting examples of baseline measurements include a baseline symptom (e.g. a liver disorder symptom) measurement, a baseline protective phenotype measurement, a baseline target oligonucleotide (e.g. mRNA) measurement or a baseline target protein measurement.

In some embodiments, the baseline measurement is obtained directly from the subject. In some embodiments, the baseline measurement is obtained by observation, for example by observation of the subject or of the subject's tissue. In some embodiments, the baseline measurement is obtained noninvasively using an imaging device. In some embodiments, the baseline measurement is obtained in a sample from the subject. In some embodiments, the baseline measurement is obtained in one or more histological tissue sections. In some embodiments, the baseline measurement is obtained by performing an assay such as an immunoassay, a colorimetric assay, or a fluorescence assay, on the sample obtained from the subject. In some embodiments, the baseline measurement is obtained by an immunoassay, a colorimetric assay, or a fluorescence assay. In some embodiments, the baseline measurement is obtained by PCR.

In some embodiments, the baseline measurement is a baseline symptom measurement. The symptom may be a symptom of a disorder associated with a target oligonucleotide. The disorder may be a liver disorder.

In some embodiments, the baseline measurement is a baseline protective phenotype measurement. The protective phenotype may protect a subject from having a disorder associated with a target oligonucleotide. The protective phenotype may be inversely correlated with an incidence of the disorder.

In some embodiments, the baseline measurement is a baseline target protein measurement. In some embodiments, the baseline target protein measurement comprises a baseline target protein level. In some embodiments, the baseline target protein level is indicated as a mass or percentage of target protein per sample weight. In some embodiments, the baseline target protein level is indicated as a mass or percentage of target protein per sample volume. In some embodiments, the baseline target protein level is indicated as a mass or percentage of target protein per total protein within the sample. In some embodiments, the baseline target protein measurement is a baseline liver or hepatocyte target protein measurement. In some embodiments, the baseline target protein measurement is a baseline circulating target protein measurement. In some embodiments, the baseline target protein measurement is obtained by an assay such as an immunoassay, a colorimetric assay, or a fluorescence assay.

In some embodiments, the baseline measurement is a baseline target mRNA measurement. In some embodiments, the baseline target mRNA measurement comprises a baseline target mRNA level. In some embodiments, the baseline target mRNA level is indicated as a mass or percentage of target mRNA per sample weight. In some embodiments, the baseline target mRNA level is indicated as a mass or percentage of target mRNA per sample volume. In some embodiments, the baseline target mRNA level is indicated as a mass or percentage of target mRNA per total mRNA within the sample. In some embodiments, the baseline target mRNA level is indicated as a mass or percentage of target mRNA per total nucleic acids within the sample. In some embodiments, the baseline target mRNA level is indicated relative to another mRNA level, such as an mRNA level of a housekeeping gene, within the sample. In some embodiments, the baseline target mRNA measurement is a baseline liver or hepatocyte target mRNA measurement. In some embodiments, the baseline target mRNA measurement is obtained by an assay such as a polymerase chain reaction (PCR) assay. In some embodiments, the PCR comprises quantitative PCR (qPCR). In some embodiments, the PCR comprises reverse transcription of the target mRNA.

Some embodiments of the methods described herein include obtaining a sample from a subject. In some embodiments, the baseline measurement is obtained in a sample obtained from the subject. In some embodiments, the sample is obtained from the subject prior to administration or treatment of the subject with a composition described herein. In some embodiments, a baseline measurement is obtained in a sample obtained from the subject prior to administering the composition to the subject. In some embodiments, the sample is obtained from the subject in a fasted state. In some embodiments, the sample is obtained from the subject after an overnight fasting period. In some embodiments, the sample is obtained from the subject in a fed state.

In some embodiments, the sample comprises a fluid. In some embodiments, the sample is a fluid sample. In some embodiments, the sample is a blood, plasma, or serum sample. In some embodiments, the sample comprises blood. In some embodiments, the sample is a blood sample. In some embodiments, the sample is a whole-blood sample. In some embodiments, the blood is fractionated or centrifuged. In some embodiments, the sample comprises plasma. In some embodiments, the sample is a plasma sample. In some embodiments, the sample comprises serum. In some embodiments, the sample is a serum sample.

In some embodiments, the sample comprises a tissue. In some embodiments, the sample is a tissue sample. In some embodiments, the sample comprises liver tissue. In some embodiments, the sample is a liver sample. In some embodiments, the sample comprises hepatocytes. In some embodiments, the sample consists of hepatocytes. For example, the baseline target mRNA measurement, or the baseline target protein measurement, may be obtained in a liver or hepatocyte sample from the patient. In some embodiments, the sample comprises adipose tissue. In some embodiments, the sample is an adipose sample. The adipose sample may comprise or consist of white adipose tissue. The adipose sample may comprise or consist of brown adipose tissue. In some embodiments, the sample comprises kidney tissue. In some embodiments, the sample is an kidney sample. In some embodiments, the sample comprises cardiac tissue such as ventricular or atrial tissue. In some embodiments, the sample is a cardiac sample. In some embodiments, the sample comprises intestinal tissue such as small intestinal tissue. In some embodiments, the sample is a small intestine sample. In some embodiments, the sample comprises lymph node tissue such as mesenteric lymph node tissue. In some embodiments, the sample is a mesenteric lymph node sample. In some embodiments, the sample comprises muscle tissue. In some embodiments, the sample is an muscle sample. In some embodiments, the tissue sample comprises liver, adipose, kidney, or cardiac tissue. In some embodiments, the tissue sample comprises brown adipose tissue, white adipose tissue, kidney tissue, intestinal tissue, mesenteric lymph node, or muscle tissue.

D. Effects

In some embodiments, the composition or administration of the composition affects a measurement such as symptom (e.g. a liver disorder symptom) measurement, a protective phenotype measurement, a target oligonucleotide (e.g. mRNA) measurement or a target protein measurement (e.g. liver tissue target protein levels), relative to the baseline measurement.

Some embodiments of the methods described herein include obtaining the measurement from a subject. For example, the measurement may be obtained from the subject after treating the subject. In some embodiments, the measurement is obtained in a second sample (such as a fluid or tissue sample described herein) obtained from the subject after the composition is administered to the subject. In some embodiments, the measurement is an indication that the disorder has been treated.

In some embodiments, the measurement is obtained directly from the subject. In some embodiments, the measurement is obtained noninvasively using an imaging device. In some embodiments, the measurement is obtained in a second sample from the subject. In some embodiments, the measurement is obtained in one or more histological tissue sections. In some embodiments, the measurement is obtained by performing an assay on the second sample obtained from the subject. In some embodiments, the measurement is obtained by an assay, such as an assay described herein. In some embodiments, the assay is an immunoassay, a colorimetric assay, a fluorescence assay, or a PCR assay. In some embodiments, the measurement is obtained by an assay such as an immunoassay, a colorimetric assay, or a fluorescence assay. In some embodiments, the measurement is obtained by PCR. In some embodiments, the measurement is obtained by histology. In some embodiments, the measurement is obtained by observation. In some embodiments, additional measurements are made, such as in a 3rd sample, a 4th sample, or a fifth sample.

In some embodiments, the measurement is obtained within 1 hour, within 2 hours, within 3 hours, within 4 hours, within 5 hours, within 6 hours, within 12 hours, within 18 hours, or within 24 hours after the administration of the composition. In some embodiments, the measurement is obtained within 1 day, within 2 days, within 3 days, within 4 days, within 5 days, within 6 days, or within 7 days after the administration of the composition. In some embodiments, the measurement is obtained within 1 week, within 2 weeks, within 3 weeks, within 1 month, within 2 months, within 3 months, within 6 months, within 1 year, within 2 years, within 3 years, within 4 years, or within 5 years after the administration of the composition. In some embodiments, the measurement is obtained after 1 hour, after 2 hours, after 3 hours, after 4 hours, after 5 hours, after 6 hours, after 12 hours, after 18 hours, or after 24 hours after the administration of the composition. In some embodiments, the measurement is obtained after 1 day, after 2 days, after 3 days, after 4 days, after 5 days, after 6 days, or after 7 days after the administration of the composition. In some embodiments, the measurement is obtained after 1 week, after 2 weeks, after 3 weeks, after 1 month, after 2 months, after 3 months, after 6 months, after 1 year, after 2 years, after 3 years, after 4 years, or after 5 years, following the administration of the composition.

In some embodiments, the composition reduces the symptom measurement relative to the baseline symptom measurement. In some embodiments, the reduction is measured in a second tissue sample obtained from the subject after administering the composition to the subject. In some embodiments, the reduction is measured directly in the subject after administering the composition to the subject. In some embodiments, the symptom measurement is decreased by about 2.5% or more, about 5% or more, or about 7.5% or more, relative to the baseline symptom measurement. In some embodiments, the symptom measurement is decreased by about 10% or more, relative to the baseline symptom measurement. In some embodiments, the symptom measurement is decreased by about 20% or more, about 30% or more, about 40% or more, about 50% or more, about 60% or more, about 70% or more, about 80% or more, about 90% or more, relative to the baseline symptom measurement. In some embodiments, the symptom measurement is decreased by no more than about 2.5%, no more than about 5%, or no more than about 7.5%, relative to the baseline symptom measurement. In some embodiments, the symptom measurement is decreased by no more than about 10%, relative to the baseline symptom measurement. In some embodiments, the symptom measurement is decreased by no more than about 20%, no more than about 30%, no more than about 40%, no more than about 50%, no more than about 60%, no more than about 70%, no more than about 80%, no more than about 90%, or no more than about 100% relative to the baseline symptom measurement. In some embodiments, the symptom measurement is decreased by 2.5%, 5%, 7.5%, 10%, 20%, 30%, 40%, 50%, 60%, 70%, 80%, 90%, or 100%, or by a range defined by any of the two aforementioned percentages.

In some embodiments, the composition increases the protective phenotype measurement relative to the baseline protective phenotype measurement. In some embodiments, the increase is measured in a second tissue sample obtained from the subject after administering the composition to the subject. In some embodiments, the increase is measured directly in the subject after administering the composition to the subject. In some embodiments, the protective phenotype measurement is increased by about 2.5% or more, about 5% or more, or about 7.5% or more, relative to the baseline protective phenotype measurement. In some embodiments, the protective phenotype measurement is increased by about 10% or more, relative to the baseline protective phenotype measurement. In some embodiments, the protective phenotype measurement is increased by about 20% or more, about 30% or more, about 40% or more, about 50% or more, about 60% or more, about 70% or more, about 80% or more, about 90% or more, relative to the baseline protective phenotype measurement. In some embodiments, the protective phenotype measurement is increased by about 100% or more, increased by about 250% or more, increased by about 500% or more, increased by about 750% or more, or increased by about 1000% or more, relative to the baseline protective phenotype measurement. In some embodiments, the protective phenotype measurement is increased by no more than about 2.5%, no more than about 5%, or no more than about 7.5%, relative to the baseline protective phenotype measurement. In some embodiments, the protective phenotype measurement is increased by no more than about 10%, relative to the baseline protective phenotype measurement. In some embodiments, the protective phenotype measurement is increased by no more than about 20%, no more than about 30%, no more than about 40%, no more than about 50%, no more than about 60%, no more than about 70%, no more than about 80%, no more than about 90%, or no more than about 100% relative to the baseline protective phenotype measurement. In some embodiments, the protective phenotype measurement is increased by no more than about 100%, increased by no more than about 250%, increased by no more than about 500%, increased by no more than about 750%, or increased by no more than about 1000%, relative to the baseline protective phenotype measurement. In some embodiments, the protective phenotype measurement is increased by 2.5%, 5%, 7.5%, 10%, 20%, 30%, 40%, 50%, 60%, 70%, 80%, 90%, 100%, 250%, 500%, 750%, or 1000%, or by a range defined by any of the two aforementioned percentages.

In some embodiments, the measurement is a target protein measurement. In some embodiments, the target protein measurement comprises a target protein level. In some embodiments, the target protein level is indicated as a mass or percentage of target protein per sample weight. In some embodiments, the target protein level is indicated as a mass or percentage of target protein per sample volume. In some embodiments, the target protein level is indicated as a mass or percentage of target protein per total protein within the sample. In some embodiments, the target protein measurement is a cell (e.g. hepatocyte) target protein measurement. In some embodiments, the target protein measurement is a tissue (e.g. liver tissue) target protein measurement. In some embodiments, the target protein measurement is a circulating target protein measurement. In some embodiments, the baseline target protein measurement is obtained by an assay such as an immunoassay, a colorimetric assay, or a fluorescence assay.

In some embodiments, the composition reduces the target protein measurement relative to the baseline target protein measurement. In some embodiments, the composition reduces tissue target protein levels (such as, but not limited to, liver tissue target protein levels) relative to the baseline target protein measurement. In some embodiments, the composition reduces cell target protein levels (such as, but not limited to, hepatocyte target protein levels) relative to the baseline target protein measurement. In some embodiments, the composition reduces circulating target protein levels relative to the baseline target protein measurement. In some embodiments, the reduced target protein levels are measured in a second sample obtained from the subject after administering the composition to the subject.

In some embodiments, the target protein measurement is decreased by about 2.5% or more, about 5% or more, or about 7.5% or more, relative to the baseline target protein measurement. In some embodiments, the target protein measurement is decreased by about 10% or more, relative to the baseline target protein measurement. In some embodiments, the target protein measurement is decreased by about 20% or more, about 30% or more, about 40% or more, about 50% or more, about 60% or more, about 70% or more, about 80% or more, about 90% or more, or about 100% relative to the baseline target protein measurement. In some embodiments, the target protein measurement is decreased by no more than about 2.5%, no more than about 5%, or no more than about 7.5%, relative to the baseline target protein measurement. In some embodiments, the target protein measurement is decreased by no more than about 10%, relative to the baseline target protein measurement. In some embodiments, the target protein measurement is decreased by no more than about 20%, no more than about 30%, no more than about 40%, no more than about 50%, no more than about 60%, no more than about 70%, no more than about 80%, no more than about 90%, or about 100% relative to the baseline target protein measurement. In some embodiments, the target protein measurement is decreased by 2.5%, 5%, 7.5%, 19%, 20%, 30%, 40%, 50%, 60%, 70%, 80%, 90%, or 100%, or by a range defined by any of the two aforementioned percentages.

In some embodiments, the measurement is a target mRNA measurement. In some embodiments, the target mRNA measurement comprises a target mRNA level. In some embodiments, the target mRNA level is indicated as a mass or percentage of target mRNA per sample weight. In some embodiments, the target mRNA level is indicated as a mass or percentage of target mRNA per sample volume. In some embodiments, the target mRNA level is indicated as a mass or percentage of target mRNA per total mRNA within the sample. In some embodiments, the target mRNA level is indicated as a mass or percentage of target mRNA per total nucleic acids within the sample. In some embodiments, the target mRNA level is indicated relative to another mRNA level, such as an mRNA level of a housekeeping gene, within the sample. In some embodiments, the target mRNA measurement is obtained by an assay such as a PCR assay. In some embodiments, the PCR comprises qPCR. In some embodiments, the PCR comprises reverse transcription of the target mRNA.

In some embodiments, the composition reduces the target mRNA measurement relative to the baseline target mRNA measurement. In some embodiments, the target mRNA measurement is obtained in a second sample obtained from the subject after administering the composition to the subject. In some embodiments, the composition reduces target mRNA levels relative to the baseline target mRNA levels. In some embodiments, the reduced target mRNA levels are measured in a second sample obtained from the subject after administering the composition to the subject. In some embodiments, the second sample is a second liver sample. In some embodiments, the second sample is second hepatocyte sample.

In some embodiments, the target mRNA measurement is reduced by about 2.5% or more, about 5% or more, or about 7.5% or more, relative to the baseline target mRNA measurement. In some embodiments, the target mRNA measurement is decreased by about 10% or more, relative to the baseline target mRNA measurement. In some embodiments, the target mRNA measurement is decreased by about 20% or more, about 30% or more, about 40% or more, about 50% or more, about 60% or more, about 70% or more, about 80% or more, about 90% or more, or about 100% relative to the baseline target mRNA measurement. In some embodiments, the target mRNA measurement is decreased by no more than about 2.5%, no more than about 5%, or no more than about 7.5%, relative to the baseline target mRNA measurement. In some embodiments, the target mRNA measurement is decreased by no more than about 10%, relative to the baseline target mRNA measurement. In some embodiments, the target mRNA measurement is decreased by no more than about 20%, no more than about 30%, no more than about 40%, no more than about 50%, no more than about 60%, no more than about 70%, no more than about 80%, no more than about 90%, or about 100% relative to the baseline target mRNA measurement. In some embodiments, the target mRNA measurement is decreased by 2.5%, 5%, 7.5%, 10%, 20%, 30%, 40%, 50%, 60%, 70%, 80%, 90%, 100%, or by a range defined by any of the two aforementioned percentages.

### III. DEFINITIONS

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

Throughout this application, various embodiments may be presented in a range format. It should be understood that the description in range format is merely for convenience and brevity and should not be construed as an inflexible limitation on the scope of the disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

As used in the specification and claims, the singular forms “a”, “an” and “the” include plural references unless the context clearly dictates otherwise. For example, the term “a sample” includes a plurality of samples, including mixtures thereof.

The terms “determining,” “measuring,” “evaluating,” “assessing,” “assaying,” and “analyzing” are often used interchangeably herein to refer to forms of measurement. The terms include determining if an element is present or not (for example, detection). These terms can include quantitative, qualitative or quantitative and qualitative determinations. Assessing can be relative or absolute. “Detecting the presence of” can include determining the amount of something present in addition to determining whether it is present or absent depending on the context.

The terms “subject,” and “patient” may be used interchangeably herein. A “subject” can be a biological entity containing expressed genetic materials. The biological entity can be a plant, animal, or microorganism, including, for example, bacteria, viruses, fungi, and protozoa. The subject can be a mammal. The mammal can be a human. The subject may be diagnosed or suspected of being at high risk for a disease. In some cases, the subject is not necessarily diagnosed or suspected of being at high risk for the disease.

As used herein, the term “about” a number refers to that number plus or minus 10% of that number. The term “about” a range refers to that range minus 10% of its lowest value and plus 10% of its greatest value.

As used herein, the terms “treatment” or “treating” are used in reference to a pharmaceutical or other intervention regimen for obtaining beneficial or desired results in the recipient. Beneficial or desired results include but are not limited to a therapeutic benefit and/or a prophylactic benefit. A therapeutic benefit may refer to eradication or amelioration of symptoms or of an underlying disorder being treated. Also, a therapeutic benefit can be achieved with the eradication or amelioration of one or more of the physiological symptoms associated with the underlying disorder such that an improvement is observed in the subject, notwithstanding that the subject may still be afflicted with the underlying disorder. A prophylactic effect includes delaying, preventing, or eliminating the appearance of a disease or condition, delaying or eliminating the onset of symptoms of a disease or condition, slowing, halting, or reversing the progression of a disease or condition, or any combination thereof. For prophylactic benefit, a subject at risk of developing a particular disease, or to a subject reporting one or more of the physiological symptoms of a disease may undergo treatment, even though a diagnosis of this disease may not have been made.

The section headings used herein are for organizational purposes only and are not to be construed as limiting the subject matter described.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicyclic rings, e.g., 5 to 12-membered spiro bicycles, such as 2-oxa-6-azaspiro[3.3]heptane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, benzo[d]thiazolyl, benzothiadiazolyl, benzo[b][1,4]dioxepinyl, benzo[b][1,4]oxazinyl, 1,4-benzodioxanyl, benzonaphthofuranyl, benzoxazolyl, benzodioxolyl, benzodioxinyl, benzopyranyl, benzopyranonyl, benzofuranyl, benzofuranonyl, benzothienyl (benzothiophenyl), benzothieno[3,2-d]pyrimidinyl, benzotriazolyl,benzo[4,6]imidazo[1,2-a]pyridinyl, carbazolyl, cinnolinyl, cyclopenta[d]pyrimidinyl, 6,7-dihydro-5H-cyclopenta[4,5]thieno[2,3-d]pyrimidinyl, 5,6-dihydrobenzo[h]quinazolinyl, 5,6-dihydrobenzo[h]cinnolinyl, 6,7-dihydro-5H-benzo[6,7]cyclohepta[1,2-c]pyridazinyl, dibenzofuranyl, dibenzothiophenyl, furanyl, furanonyl, furo[3,2-c]pyridinyl, 5,6,7,8,9,10-hexahydrocycloocta[d]pyrimidinyl, 5,6,7,8,9,10-hexahydrocycloocta[d]pyridazinyl, 5,6,7,8,9,10-hexahydrocycloocta[d]pyridinyl, isothiazolyl, imidazolyl, indazolyl, indolyl, indazolyl, isoindolyl, indolinyl, isoindolinyl, isoquinolyl, indolizinyl, isoxazolyl, 5,8-methano-5,6,7,8-tetrahydroquinazolinyl, naphthyridinyl, 1,6-naphthyridinonyl, oxadiazolyl, 2-oxoazepinyl, oxazolyl, oxiranyl, 5,6,6a, 7,8,9,10,10a-octahydrobenzo[h]quinazolinyl, 1-phenyl-1H-pyrrolyl, phenazinyl, phenothiazinyl, phenoxazinyl, phthalazinyl, pteridinyl, purinyl, pyrrolyl, pyrazolyl, pyrazolo[3,4-d]pyrimidinyl, pyridinyl, pyrido[3,2-d]pyrimidinyl, pyrido[3,4-d]pyrimidinyl, pyrazinyl, pyrimidinyl, pyridazinyl, pyrrolyl, quinazolinyl, quinoxalinyl, quinolinyl, isoquinolinyl, tetrahydroquinolinyl, 5,6,7,8-tetrahydroquinazolinyl, 5,6,7,8-tetrahydrobenzo[4,5]thieno[2,3-d]pyrimidinyl, 6,7,8,9-tetrahydro-5H-cyclohepta[4,5]thieno[2,3-d]pyrimidinyl, 5,6,7,8-tetrahydropyrido[4,5-c]pyridazinyl, thiazolyl, thiadiazolyl, triazolyl, tetrazolyl, triazinyl, thieno[2,3-d]pyrimidinyl, thieno[3,2-d]pyrimidinyl, thieno[2,3-c]pyridinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatomsin the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, 2-oxa-6-azaspiro[3.3]heptane, and 1,1-dioxo-thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R is a straight or branched alkylene, alkenylene or alkynylene chain.

Double bonds to oxygen atoms, such as oxo groups, are represented herein as both “═O” and “(O)”. Double bonds to nitrogen atoms are represented as both“=NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

### VI. NUMBERED EMBODIMENTS

Any aspect of the following may be included in an embodiment:

1. A compound represented by Formula (I):

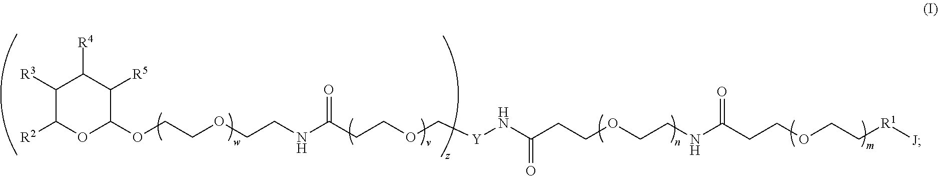

or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
if z is 3, Y is C
if z is 2, Y is CR⁶, or
if z is 1, Y is C(R⁶)₂;
   R¹ is a linker selected from:
—O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
   each R² is independently selected from:
C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   R³ and R⁴ are each independently selected from:
—OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   each R⁵ is independently selected from:
—OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
   each R⁶ is independently selected from:
hydrogen;
halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
C₁₋₆ alkyl optionally substituted with one or more substituents independently selected from halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
   each R⁷ is independently selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆haloalkyl.

2. The compound or salt of embodiment 1, wherein each w is independently selected from any value from 1 to 10.

3. The compound or salt of any one of embodiments 1 to 2, wherein each w is independently selected from any value from 1 to 5.

4. The compound or salt of any one of embodiments 1 to 3, wherein each w is 1.

5. The compound or salt of any one of embodiments 1 to 4, wherein each v is independently selected from any value from 1 to 10.

6. The compound or salt of any one of embodiments 1 to 5, wherein each v is independently selected from any value from 1 to 5.

7. The compound or salt of any one of embodiments 1 to 6, wherein each v is 1.

8. The compound or salt of any one of embodiments 1 to 7, wherein n is selected from any value from 1 to 10.

9. The compound or salt of any one of embodiments 1 to 8, wherein n is selected from any value from 1 to 5.

10. The compound or salt of any one of embodiments 1 to 9, wherein n is 2.

11. The compound or salt of any one of embodiments 1 to 10, wherein m is selected from any value from 1 to 10.

12. The compound or salt of any one of embodiments 1 to 11, wherein m is selected from any value from 1 to 5.

13. The compound or salt of any one of embodiments 1 to 12, wherein m is 4.

14. The compound or salt of any one of embodiments 1 to 13, wherein z is 3 and Y is C.

15. The compound or salt of any one of embodiments 1 to 14, wherein R¹ is —OP(O)(OR⁷)O—.

16. The compound or salt of any one of embodiments 1 to 15, wherein R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷.

17. The compound or salt of any one of embodiments 1 to 16, wherein R² is selected from C₁₋₃ alkyl substituted with one or more substituents independently selected from —OR⁷, —OC(O)R⁷, —SR⁷, and —N(R⁷)₂.

18. The compound or salt of any one of embodiments 1 to 17, wherein R³ is selected from halogen, —OR⁷, —SR⁷, —N(R⁷)₂, —C(O)R⁷, —OC(O)R⁷, and —S(O)R⁷.

19. The compound or salt of any one of embodiments 1 to 18, wherein R³ is selected from —OR⁷, —SR⁷, —OC(O)R⁷, and —N(R⁷)₂.

20. The compound or salt of any one of embodiments 1 to 19, wherein R⁴ is selected from halogen, —OR⁷, —SR⁷, —N(R⁷)₂, —C(O)R⁷, —OC(O)R⁷, and —S(O)R⁷.

21. The compound or salt of any one of embodiments 1 to 20, wherein R⁴ is selected from —OR⁷, —SR⁷, —OC(O)R⁷, and —N(R⁷)₂.

22. The compound or salt of any one of embodiments 1 to 21, wherein R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷.

23. The compound or salt of any one of embodiments 1 to 22, wherein R⁵ is selected from —OC(O)R⁷ and —N(R⁷)C(O)R⁷.

24. The compound or salt of any one of embodiments 1 to 23, wherein each R⁷ is independently selected from:
hydrogen; and
C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle.

25. The compound or salt of any one of embodiments 1 to 24, wherein each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl).

26. The compound of any one of embodiments 1-25, wherein the compound comprises:

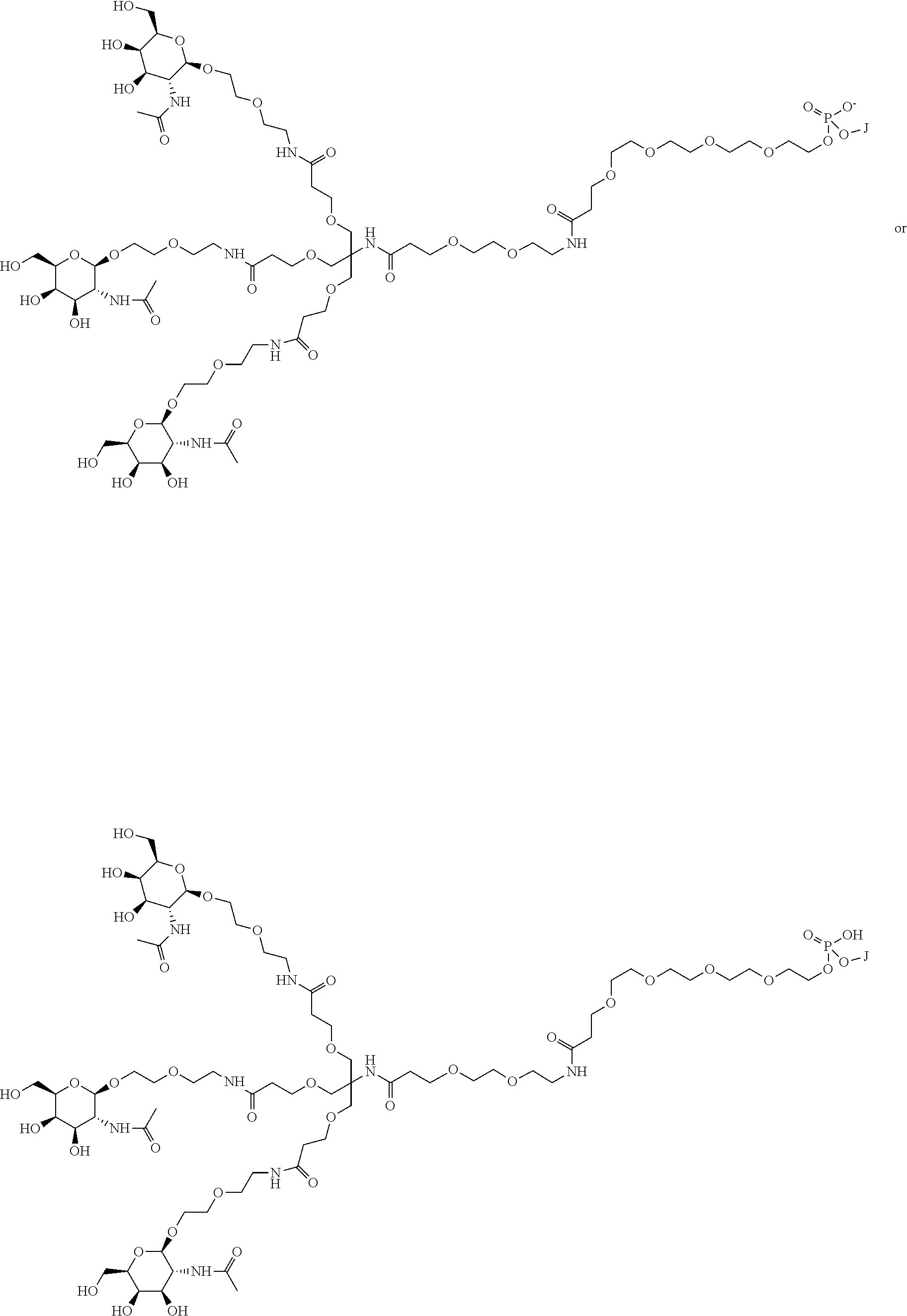

27. The compound or salt of embodiment 1 represented by Formula (II):

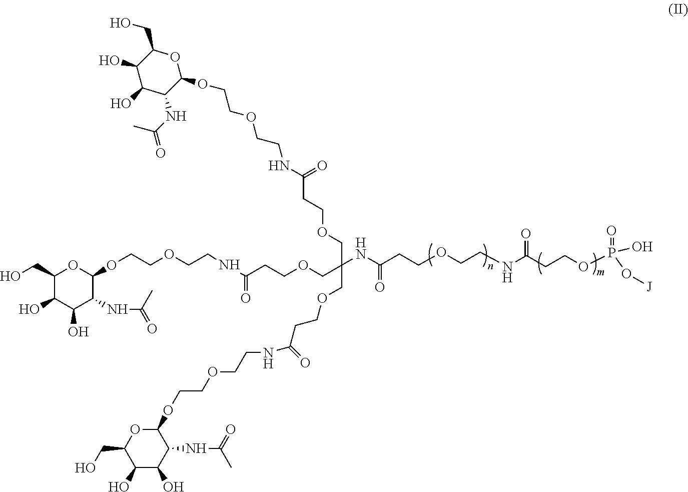

wherein
J is an oligonucleotide;
n is selected from any value from 1 to 10; and
m is selected from any value from 1 to 10.

28. The compound or salt of embodiment 27, wherein n is 2.

29. The compound or salt of embodiment 27 or 28, wherein m is 4.

30. The compound of any one of embodiments 1 to 29, wherein the oligonucleotide (J) is attached at a 5′ end or a 3′ end of the oligonucleotide.

31. The compound of any one of embodiments 1 to 30, wherein the oligonucleotide comprises DNA.

32. The compound of any one of embodiments 1 to 31, wherein the oligonucleotide comprises RNA.

33. The compound of any one of embodiments 1 to 32, wherein the oligonucleotide comprises one or more modified internucleoside linkages.

34. The compound of embodiment 33, wherein the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

35. The compound of any one of embodiments 1 to 34, wherein the oligonucleotide comprises 1, 2, 3, 4, 5, 6, 7, 8, 9, 10, 11, 12, 13, 14, 15, 16, 17, 18, 19, or 20 modified internucleoside linkages.

36. The compound of any one of embodiments 1 to 35, wherein the oligonucleotide comprises one or more modified nucleosides.

37. The compound of embodiment 36, wherein the one or more modified nucleosides comprise a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

38. The compound of embodiment 36 or 37, wherein the one or more modified nucleosides comprise a 2′,4′ constrained ethyl nucleoside, a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, 2′-ara-F, 2′fluoro, or 2′ O-alkyl, or a combination thereof.

39. The compound of any one of embodiments 1 to 38, wherein the oligonucleotide comprises 1, 2, 3, 4, 5, 6, 7, 8, 9, 10, 11, 12, 13, 14, 15, 16, 17, 18, 19, 20, 21, or more modified nucleosides.

40. The compound of any one of embodiments 1 to 39, wherein the oligonucleotide comprises a lipid attached at a 3′ or 5′ terminus of the oligonucleotide.

41. The compound of embodiment 40, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof.

42. The compound of any one of embodiments 1 to 41, wherein the oligonucleotide comprises an arginine-glycine-aspartic acid (RGD) peptide attached at a 3′ or 5′ terminus of the oligonucleotide.

43. The compound of embodiment 42, wherein the RGD peptide comprises Cyclo(-Arg-Gly-Asp-D-Phe-Cys), Cyclo(-Arg-Gly-Asp-D-Phe-Lys), Cyclo(-Arg-Gly-Asp-D-Phe-azido), an amino benzoic acid derived RGD, or a combination thereof.

44. The compound of any one of embodiments 1 to 43, wherein the oligonucleotide comprises a small interfering RNA (siRNA) comprising a sense strand and an antisense strand.

45. The compound of embodiment 44, wherein the sense strand is 12-30 nucleosides in length.

46. The compound of embodiment 44 or 45, wherein the antisense strand is 12-30 nucleosides in length.

47. The compound of any one of embodiments 44 to 46, wherein the sense strand and the antisense strand form a double-stranded RNA duplex.

48. The compound of embodiment 47, wherein a first base pair of the double-stranded RNA duplex is an AU base pair.

49. The compound of any one of embodiments 44 to 48, wherein the sense strand or the antisense strand comprises a 3′ overhang.

50. The compound of embodiment 49, wherein the 3′ overhang comprises 1, 2, or more nucleosides.

51. The compound of any one of embodiments 44 to 50, wherein the sense strand comprises any one of modification patterns 1 S to 6S, or 1S #2 to 6S #2.

52. The compound of any one of embodiments 44 to 51, wherein the antisense strand comprises any one of modification patterns 1AS to 9AS.

53. The compound of any one of embodiments 1 to 43, wherein the oligonucleotide comprises an antisense oligonucleotide (ASO).

54. The compound of embodiment 53, wherein the ASO is 12-30 nucleosides in length.

55. The compound of embodiment 53 or 54, wherein the ASO comprises modification pattern ASO1.

56. The compound of any one of embodiments 1 to 55, wherein the compound binds to an asialoglycoprotein receptor.

57. The compound of any one of embodiments 1 to 56, wherein the compound targets a hepatocyte.

58. A pharmaceutical composition comprising the compound of any one of embodiments 1 to 57, and a pharmaceutically acceptable carrier, excipient, or diluent.

59. The pharmaceutical composition of embodiment 58, wherein the pharmaceutical composition is sterile.

60. The pharmaceutical composition of embodiment 58 or 59, wherein the pharmaceutical composition comprises a pharmaceutically acceptable carrier.

61. The pharmaceutical composition of embodiment 60, wherein the pharmaceutically acceptable carrier comprises water, a buffer, or a saline solution.

62. The pharmaceutical composition of any one of embodiments 58 to 61, wherein the oligonucleotide targets a target mRNA and when administered to a subject in an effective amount decreases the target mRNA or a target protein by at least 10%.

63. A method of decreasing a target mRNA or target protein in a subject in need thereof, comprising administering an effective amount of the pharmaceutical composition of any one of embodiments 58 to 62 to the subject.

64. The method of embodiment 63, wherein the effective amount decreases a measurement of the target mRNA or target protein in the subject, relative to a baseline target mRNA or target protein measurement.

65. The method of embodiment 63 or 64, wherein the effective amount treats a disorder in the subject.

66. The method of embodiment 65, wherein the effective amount decreases a measurement of a symptom or parameter related to the disorder in the subject, relative to a baseline symptom or parameter measurement.

67. The method of embodiment 65 or 66, wherein the disorder comprises a metabolic disorder.

68. The method of any one of embodiments 65 to 67, wherein the disorder comprises a liver disorder.

### VII. EXAMPLES

### Example 1: Identification of Variants in a Target Oligonucleotide Associated with Increased or Decreased Risk of a Disorder

Approximately 30,000,000 imputed variants are to be analyzed in ˜375,000 individuals from a Biobank cohort for associations with liver disorders such as non-alcoholic steatohepatitis.

Protective or maladaptive associations are observed between specific allelic variants of various target genes and liver diseases. The associations will suggest that in some cases therapeutic inhibition or modulation of a target protein encoded by any of the target genes may be an effective genetically-informed method of treatment for any of the liver disorders.

### Example 2: Bioinformatic Selection of Sequences in Order to Identify Therapeutic siRNAs to Downmodulate Expression of a Target mRNA

Screening sets are to be defined based on bioinformatic analysis. Therapeutic siRNAs are designed to bind human target mRNAs, and the target mRNA sequence of at least one toxicology-relevant species such as a non-human primate (NHP) species (e.g. a rhesus or cynomolgus monkey). Drivers for the design of the screening set are predicted specificity of the siRNAs against the transcriptome of the relevant species as well as cross-reactivity between species. Predicted specificity in human, rhesus monkey, cynomolgus monkey, mouse and rat are determined for sense (S) and antisense (AS) strands. These are assigned a “specificity score” which considers the likelihood of unintended downregulation of any other transcript by full or partial complementarity of an siRNA strand (up to 4 mismatches within positions 2-18) as well as the number and positions of mismatches. Thus, off-target(s) for antisense and sense strands of each siRNA are identified. In addition, the number of potential off-targets are used as an additional specificity factor in the specificity score. As identified, siRNAs with high specificity and a low number of predicted off-targets provide a benefit of increased targeting specificity.

In addition to selecting siRNA sequences with high sequence specificity to the target mRNA, siRNA sequences within a seed region are analyzed for similarity to seed regions of known miRNAs. siRNAs can function in a miRNA like manner via base-pairing with complementary sequences within the 3′-UTR of mRNA molecules. The complementarity typically encompasses the 5′-bases at positions 2-7 of the miRNA (seed region). To circumvent siRNAs to act via functional miRNA binding sites, siRNA strands containing natural miRNA seed regions are avoided. Seed regions identified in miRNAs from human, mouse, rat, rhesus monkey, dog, rabbit and pig are referred to as “conserved”. Combining the “specificity score” with miRNA seed analysis yields a “specificity category”. This is divided into categories 1-4, with 1 having the highest specificity and 4 having the lowest specificity. Each strand of the siRNA is assigned to a specificity category.

Species cross-reactivity are assessed for human, cynomolgus monkey, rhesus monkey, mouse and rat. The analysis is based on a canonical siRNA design using 19 bases and 17 bases (without considering positions 1 and 19) for cross-reactivity. Full match as well as single mismatch analyses are included.

Analysis of the human Single Nucleotide Polymorphism (SNP) database (NCBI-DB-SNP) to identify siRNAs targeting regions with known SNPs are also carried out to identify siRNAs that may be non-functional in individuals containing the SNP. Information regarding the positions of SNPs within the target sequence as well as minor allele frequency (MAF) in case data are obtained in this analysis.

The above methods can be used to identify therapeutic siRNAs to downmodulate expression of a target mRNA. Bioinformatic methods may also be used to identify ASOs that bind and downmodulate expression of a target mRNA.

### Example 3: Chemically Modified siRNAs

siRNAs that bind a target mRNA can be synthesized with chemical modifications with the sense strand having modification pattern 1 S and the antisense strand having the pattern 1AS. In addition, adenosine can be placed at position 19 in the sense strand and uridine at position 1 in the antisense strand.

The siRNAs that bind a target mRNA can also be synthesized with chemical modifications with the sense strand having modification pattern 2S and the antisense strand having modification pattern 3AS. In addition, adenosine can be placed at position 19 in the sense strand and uridine at position 1 in the antisense strand.

The siRNAs that bind a target mRNA can also be synthesized with chemical modifications with the sense strand having modification pattern 2S and the antisense strand having modification pattern 9AS. In addition, adenosine can be placed at position 19 in the sense strand and uridine at position 1 in the antisense strand.

The siRNAs targeting that bind a target mRNA can also be synthesized with chemical modifications with the sense strand having modification pattern 3 S and the antisense strand having modification pattern 3AS. In addition, adenosine can be placed at position 19 in the sense strand and uridine at position 1 in the antisense strand.

### Example 4: Screening siRNAs for Activity in Cells in Culture

The chemically modified siRNAs derived from sequences in examples 2 and 3 will be assayed for target mRNA knockdown activity in cells in culture. A cell line that expresses the target mRNA is to be seeded in 96-well tissue culture plates at a cell density of 10,000 cells per well in DMEM supplemented with 10% fetal bovine serum and incubated overnight in a water-jacketed, humidified incubator at 37° C. in an atmosphere composed of air plus 5% carbon dioxide. The siRNAs are individually transfected into cells in duplicate wells at 10 nM final concentration using 0.3 μL Lipofectamine RNAiMax (Fisher) per well. Silencer Select Negative Control #1 (ThermoFisher, Catalog #4390843) and a positive control siRNA are transfected at 10 nM final concentration as controls. After incubation for 48 hours at 37° C., total RNA is harvested from each well and cDNA prepared using TaqMan® Fast Advanced Cells-to-CT™ Kit (ThermoFisher, Catalog #A35374) according to the manufacturer's instructions. The level of target mRNA in each well will be measured in triplicate by real-time qPCR on an Applied Biosystems 7500 Fast Real-Time PCR machine using TaqMan Gene Expression Assay for the human target mRNA. The level of PPIA mRNA will be measured using TaqMan Gene Expression Assay (ThermoFisher) and used to determine relative target mRNA levels in each well using the delta-delta Ct method. Data will be normalized to relative target mRNA levels in untreated cells.

The siRNAs showing the greatest degree of knockdown of target mRNA at 10 nM will be tested in a second screen for activity at 1 nM concentration using the transfection procedures as described above. Similar experiments may be performed using ASOs. Thus, siRNAs and ASOs may be identified that most effectively downmodulate expression of the target mRNA.

### Example 5: GalNAc Ligands for Hepatocyte Targeting of Oligonucleotides

Without limiting the disclosure to these individual methods, there are at least two general methods for attachment of multivalent N-acetylgalactosamine (GalNAc) ligands to oligonucleotides: solid or solution-phase conjugations. GalNAc ligands may be attached to solid phase resin for 3′ conjugation or at the 5′ terminus using GalNAc phosphoramidite reagents. GalNAc phosphoramidites may be coupled on solid phase as for other nucleosides in the oligonucleotide sequence at any position in the sequence. A non-limiting example of a phosphoramidite reagent for GalNAc conjugation to a 5′ end oligonucleotide is shown in Table 1.

TABLE 1
GalNAc Conjugation Reagent

| Type of | |
|---|---|
| conjugation | Structure |
| Solid phase 5′ attach- ment phosphora- midite | 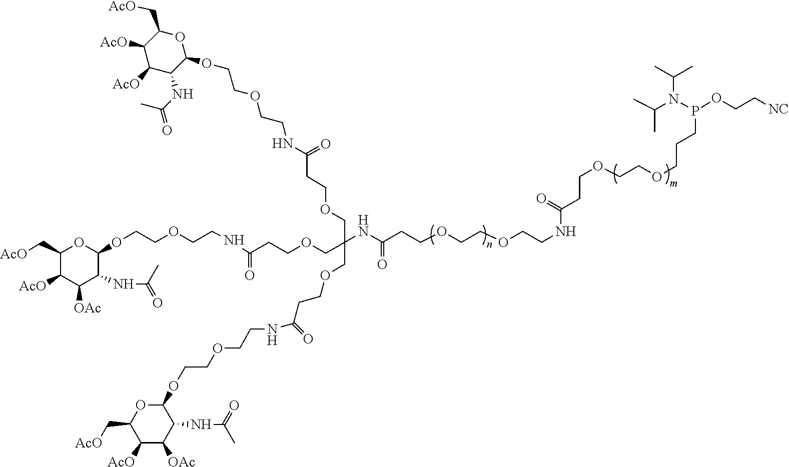 |

The following is a non-limiting example of a scheme for synthesizing a phosphoramidite in Table 1. For the phosphoramidite in Table 1, m or n may be any value from 1 to 10. For example, m may be 4, and n may be 2.

### Example 6: Synthesis of GalNAc Ligands

### Scheme for the Preparation of NAcegal-Linker-TMSOTf

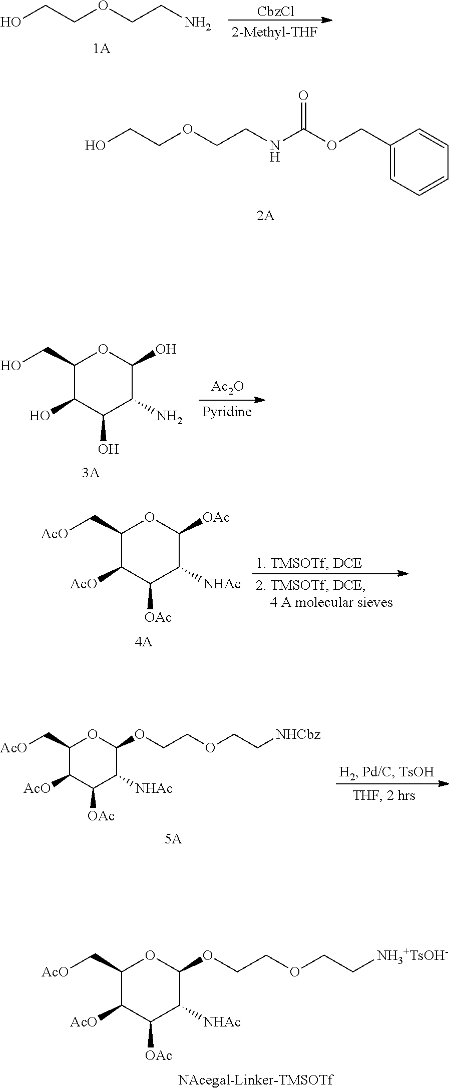

### General Procedure for Preparation of Compound 2A

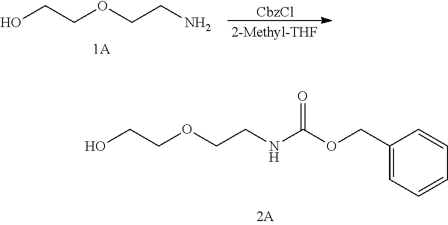

To a solution of Compound 1A (500 g, 4.76 mol, 476 mL) in 2-Methly-THF (2.00 L) was added CbzCl (406 g, 2.38 mol, 338 mL) in 2-Methly-THF (750 mL) dropwise at 0° C. The mixture was stirred at 25° C. for 2 hrs under N₂ atmosphere. TLC (DCM:MeOH=20:1, PMA) indicated CbzCl was consumed completely and one new spot (R_{f}=0.43) formed. The reaction mixture was added HCl/EtOAc (1 N, 180 mL) and stirred for 30 mins, white solid was removed by filtration through celite, the filtrate was concentrated under vacuum to give Compound 2A (540 g, 2.26 mol, 47.5% yield) as a pale yellow oil and used into the next step without further purification. ¹H NMR: δ 7.28-7.41 (m, 5H), 5.55 (br s, 1H), 5.01-5.22 (m, 2H), 3.63-3.80 (m, 2H), 3.46-3.59 (m, 4H), 3.29-3.44 (m, 2H), 2.83-3.02 (m, 1H).

### General Procedure for Preparation of Compound 4A

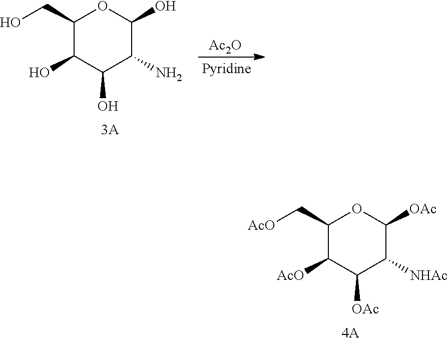 [SMILES: "Ac2O" ; N[C@@H]1[C@@H](O)[C@@H](O)[C@@H](CO)O[C@H]1O ; CC(=O)N[C@H]1[C@H](OC(C)=O)O[C@H](COC(C)=O)[C@H](OC(C)=O)[C@@H]1OC(C)=O]

To a solution of Compound 3A (1.00 kg, 4.64 mol, HCl) in pyridine (5.00 L) was added acetyl acetate (4.73 kg, 46.4 mol, 4.34 L) dropwise at 0° C. under N₂ atmosphere. The mixture was stirred at 25° C. for 16 hrs under N₂ atmosphere. TLC (DCM:MeOH=20:1, PMA) indicated Compound 3A was consumed completely and two new spots (R_{f}=0.35) formed. The reaction mixture was added to cold water (30.0 L) and stirred at 0° C. for 0.5 hr, white solid formed, filtered and dried to give Compound 4A (1.55 kg, 3.98 mol, 85.8% yield) as a white solid and used in the next step without further purification. ¹H NMR: δ 7.90 (d, J=9.29 Hz, 1H), 5.64 (d, J=8.78 Hz, 1H), 5.26 (d, J=3.01 Hz, 1H), 5.06 (dd, J=11.29, 3.26 Hz, 1H), 4.22 (t, J=6.15 Hz, 1H), 3.95-4.16 (m, 3H), 2.12 (s, 3H), 2.03 (s, 3H), 1.99 (s, 3H), 1.90 (s, 3H), 1.78 (s, 3H).

### General Procedure for Preparation of Compound 5 A

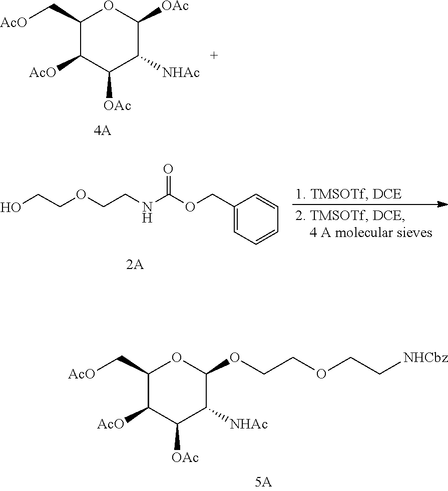

To a solution of Compound 4A (300 g, 771 mmol) in DCE (1.50 L) was added TMSOTf (257 g, 1.16 mol, 209 mL) and stirred for 2 hrs at 60° C., and then stirred for 1 hr at 25° C. Compound 2A (203 g, 848 mmol) was dissolved in DCE (1.50 L) and added 4 Å powder molecular sieves (150 g) stirring for 30 mins under N₂ atmosphere. Then the solution of Compound 4A in DCE was added dropwise to the mixture at 0° C. The mixture was stirred at 25° C. for 16 hrs under N₂ atmosphere. TLC (DCM:MeOH=25:1, PMA) indicated Compound 4A was consumed completely and new spot (R_{f}=0.24) formed. The reaction mixture was filtered and washed with sat. NaHCO₃ (2.00 L), water (2.00 L) and sat. brine (2.00 L). The organic layer was dried over anhydrous Na₂SO4, filtered and concentrated under reduced pressure to give a residue. The residue was triturated with 2-Me-THE/heptane (5/3, v/v, 1.80 L) for 2 hrs, filtered and dried to give Compound 5A (225 g, 389 mmol, 50.3% yield, 98.4% purity) as a white solid. ¹H NMR: δ 7.81 (d, J=9.29 Hz, 1H), 7.20-7.42 (m, 6H), 5.21 (d, J=3.26 Hz, 1H), 4.92-5.05 (m, 3H), 4.55 (d, J=8.28 Hz, 1H), 3.98-4.07 (m, 3H), 3.82-3.93 (m, 1H), 3.71-3.81 (m, 1H), 3.55-3.62 (m, 1H), 3.43-3.53 (m, 2H), 3.37-3.43 (m, 2H), 3.14 (q, J=5.77 Hz, 2H), 2.10 (s, 3H), 1.99 (s, 3H), 1.89 (s, 3H), 1.77 (s, 3H).

### General Procedure for Preparation of NAcegal-Linker-TMSOTf

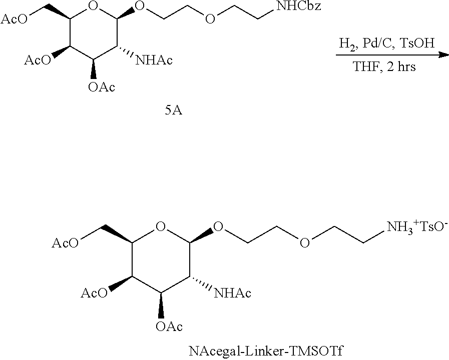

To a solution of Compound 5A (200 g, 352 mmol) in THF (1.0 L) was added dry Pd/C (15.0 g, 10% purity) and TsOH (60.6 g, 352 mmol) under N₂ atmosphere. The suspension was degassed under vacuum and purged with H₂ several times. The mixture was stirred at 25° C. for 3 hrs under H₂ (45 psi) atmosphere. TLC (DCM:MeOH=10:1, PMA) indicated Compound 5A was consumed completely and one new spot (R_{f}=0.04) was formed. The reaction mixture was filtered and concentrated (≤40° C.) under reduced pressure to give a residue. Diluted with anhydrous DCM (500 mL, dried overnight with 4 Å molecular sieves (dried at 300° C. for 12 hrs)) and concentrate to give a residue and run Karl Fisher (KF) to check for water content. This was repeated 3 times with anhydrous DCM (500 mL) dilutions and concentration to give NAcegal-Linker-TMSOTf (205 g, 95.8% yield, TsOH salt) as a foamy white solid. ¹H NMR: δ 7.91 (d, J=9.03 Hz, 1H), 7.53-7.86 (m, 2H), 7.49 (d, J=8.03 Hz, 2H), 7.13 (d, J=8.03 Hz, 2H), 5.22 (d, J=3.26 Hz, 1H), 4.98 (dd, J=11.29, 3.26 Hz, 1H), 4.57 (d, J=8.53 Hz, 1H), 3.99-4.05 (m, 3H), 3.87-3.94 (m, 1H), 3.79-3.85 (m, 1H), 3.51-3.62 (m, 5H), 2.96 (br t, J=5.14 Hz, 2H), 2.29 (s, 3H), 2.10 (s, 3H), 2.00 (s, 3H), 1.89 (s, 3H), 1.78 (s, 3H).

### Scheme for the Preparation of TRIS-PEG2-CBZ

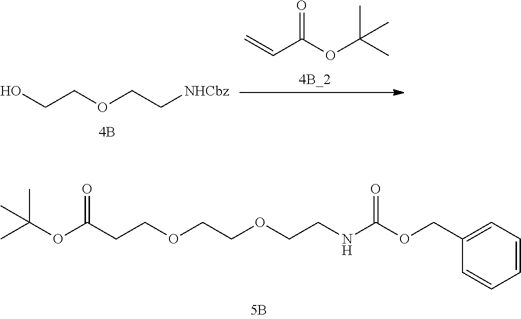

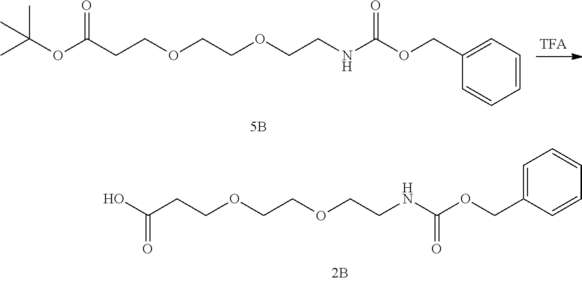 [SMILES: CC(C)(C)OC(=O)CCOCCOCCNC(=O)OCc1ccccc1 ; O=C(O)CCOCCOCCNC(=O)OCc1ccccc1]

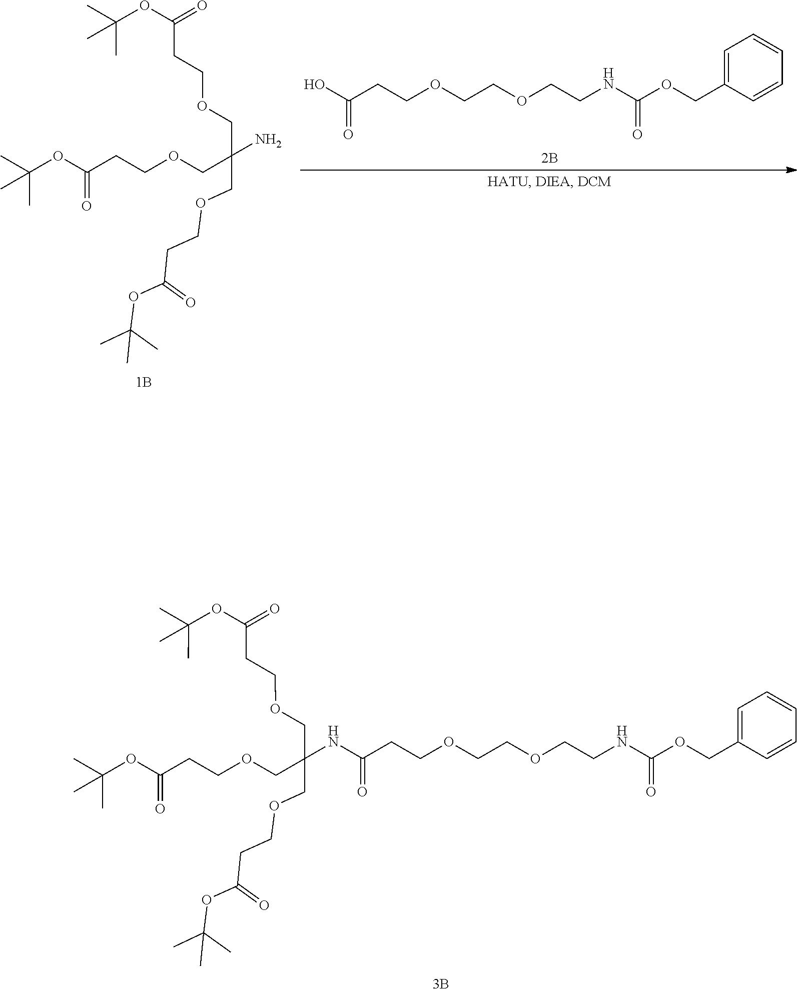 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

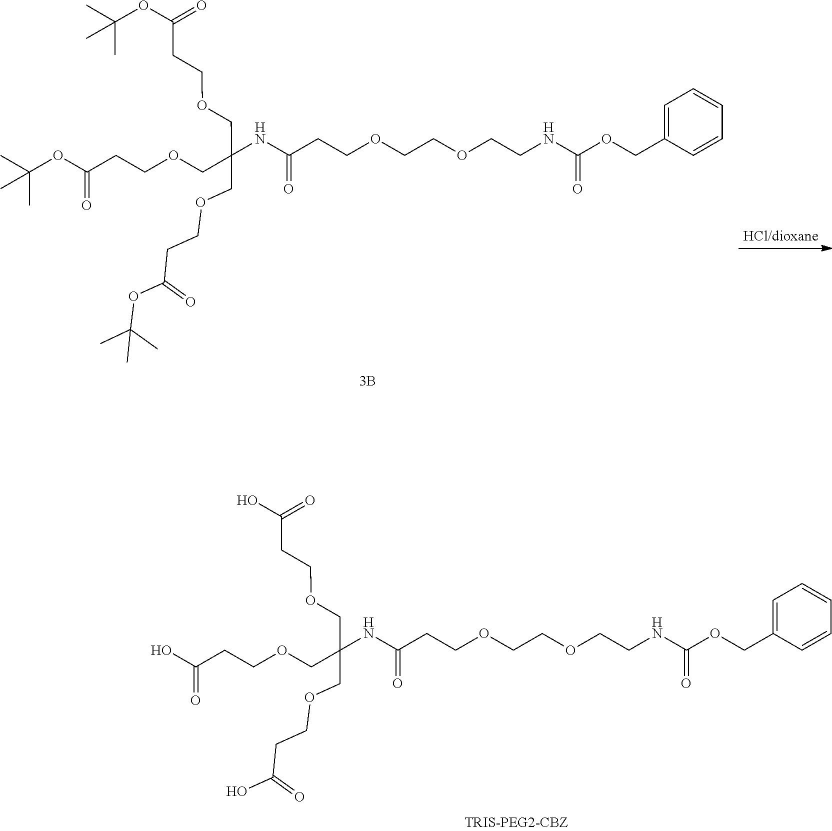 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

### General Procedure for Preparation of Compound 5B

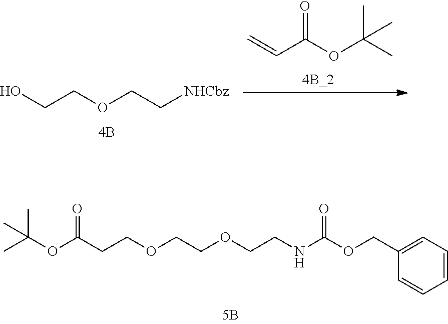

To a solution of Compound 4B (400 g, 1.67 mol, 1.00 eq) and NaOH (10 M, 16.7 mL, 0.10 eq) in THE (2.00 L) was added Compound 4B_2 (1.07 kg, 8.36 mol, 1.20 L, 5.00 eq), the mixture was stirred at 30° C. for 2 hrs. LCMS showed the desired MS was given. Five batches of solution were combined to one batch, then the mixture was diluted with water (6.00 L), extracted with ethyl acetate (3.00 L*3), the combined organic layer was washed with brine (3.00 L), dried over Na₂SO₄, filtered and concentrated under vacuum. The crude was purified by column chromatography (SiO₂, petroleum ether:ethyl acetate=100:1-10:1, R_{f}=0.5) to give Compound 5B (2.36 kg, 6.43 mol, 76.9% yield) as light yellow oil. HNMR: δ 7.31-7.36 (m, 5H), 5.38 (s, 1H), 5.11-5.16 (m, 2H), 3.75 (t, J=6.4 Hz), 3.54-3.62 (m, 6H), 3.39 (d, J=5.2 Hz), 2.61 (t, J=6.0 Hz).

### General Procedure for Preparation of 3-oxo-1-phenyl-2,7,10-trioxa-4-azatridecan-13-oic acid (Compound 2B Below)

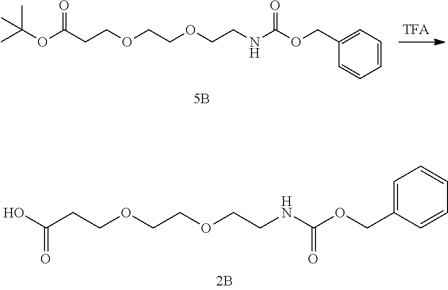 [SMILES: CC(C)(C)OC(=O)CCOCCOCCNC(=O)OCc1ccccc1 ; O=C(O)CCOCCOCCNC(=O)OCc1ccccc1]

To a solution of Compound 5B (741 g, 2.02 mol, 1.00 eq) in DCM (2.80 L) was added TFA (1.43 kg, 12.5 mol, 928 mL, 6.22 eq), the mixture was stirred at 25° C. for 3 hrs. LCMS showed the desired MS was given. The mixture was diluted with DCM (5.00 L), washed with water (3.00 L*3), brine (2.00 L), the combined organic layer was dried over Na₂SO₄, filtered and concentrated under vacuum to give Compound 2B (1800 g, crude) as light yellow oil. HNMR: δ 9.46 (s, 5H), 7.27-7.34 (m, 5H), 6.50-6.65 (m, 1H), 5.71 (s, 1H), 5.10-5.15 (m, 2H), 3.68-3.70 (m, 14H), 3.58-3.61 (m, 6H), 3.39 (s, 2H), 2.55 (s, 6H), 2.44 (s, 2H).

### General Procedure for Preparation of Compound 3B

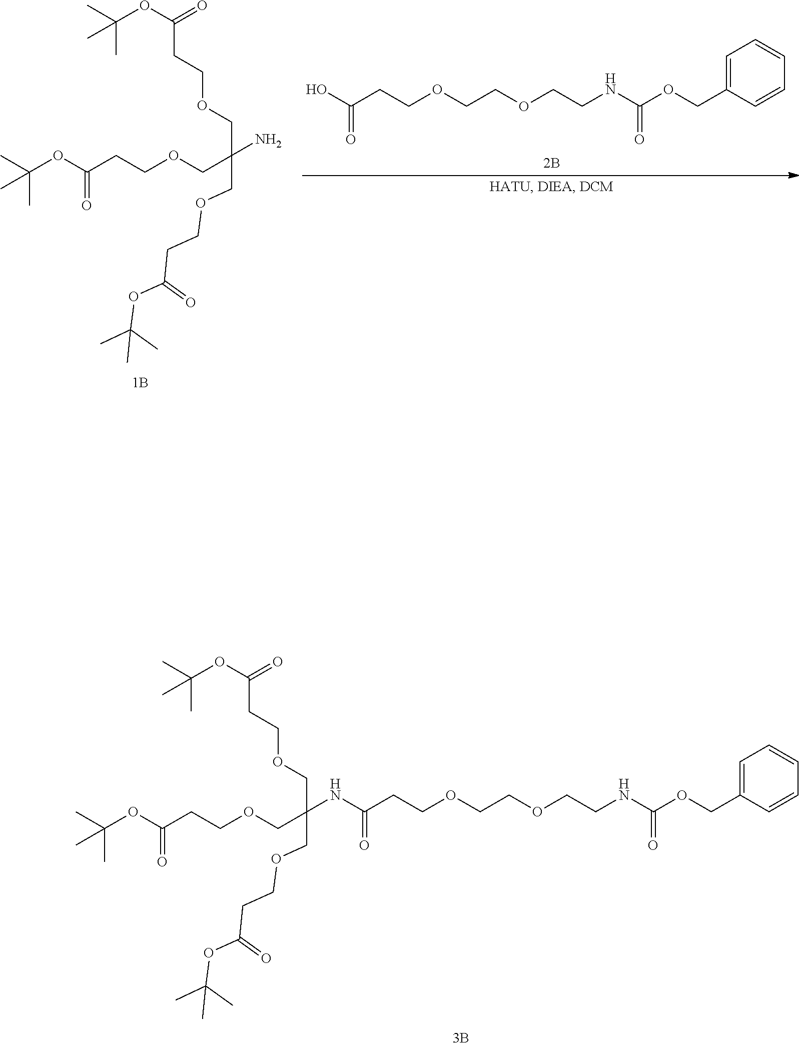 [SMILES: CC(C)(C)OC(=O)CCOCC(N)(COCCC(=O)OC(C)(C)C)COCCC(=O)OC(C)(C)C ; O=C(O)CCOCCOCCNC(=O)OCc1ccccc1 ; CC(C)(C)OC(=O)CCOCC(COCCC(=O)OC(C)(C)C)(COCCC(=O)OC(C)(C)C)NC(=O)CCOCCOCCNC(=O)OCc1ccccc1]

To a solution of Compound 2B (375 g, 999 mmol, 83.0% purity, 1.00 eq) in DCM (1.80 L) was added HATU (570 g, 1.50 mol, 1.50 eq) and DIEA (258 g, 2.00 mol, 348 mL, 2.00 eq) at 0° C., the mixture was stirred at 0° C. for 30 min, then Compound 1B (606 g, 1.20 mol, 1.20 eq) was added, the mixture was stirred at 25° C. for 1 hr. LCMS showed desired MS was given. The mixture was combined to one batch, then the mixture was diluted with DCM (5.00 L), washed with 1 N HCl aqueous solution (2.00 L*2), then the organic layer was washed with saturated Na₂CO₃ aqueous solution (2.00 L*2) and brine (2.00 L), the organic layer was dried over Na₂SO₄, filtered and concentrated under vacuum to give Compound 3B (3.88 kg, crude) as yellow oil.

### General Procedure for Preparation of TRIS-PEG2-CBZ

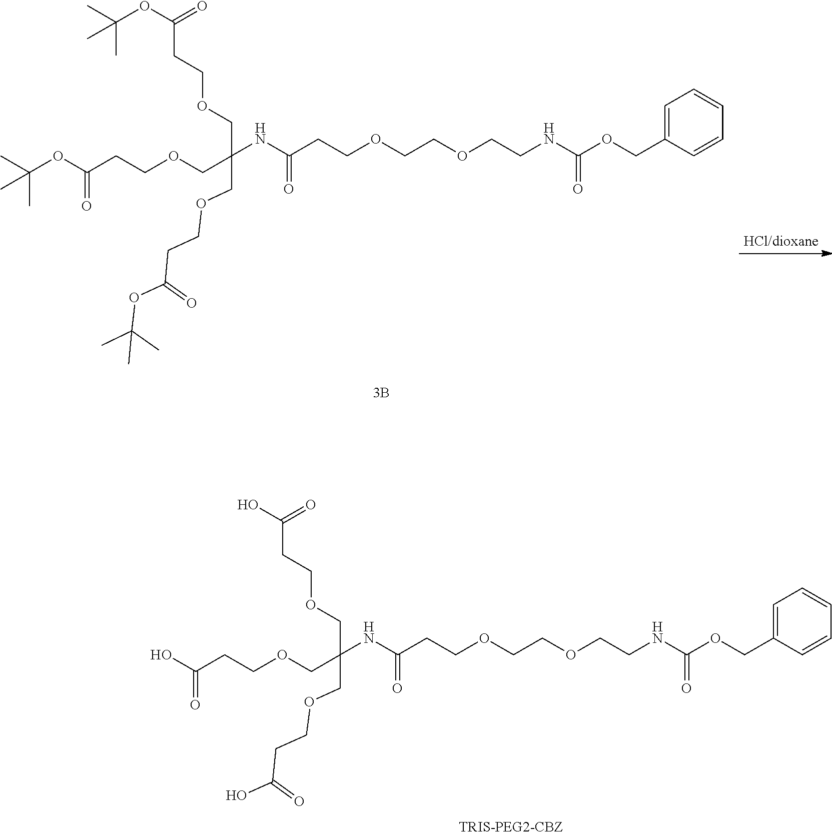 [SMILES: CC(C)(C)OC(=O)CCOCC(COCCC(=O)OC(C)(C)C)(COCCC(=O)OC(C)(C)C)NC(=O)CCOCCOCCNC(=O)OCc1ccccc1 ; O=C(O)CCOCC(COCCC(=O)O)(COCCC(=O)O)NC(=O)CCOCCOCCNC(=O)OCc1ccccc1]

A solution of Compound 3B (775 g, 487 mmol, 50.3% purity, 1.00 eq) in HCl/dioxane (4 M, 2.91 L, 23.8 eq) was stirred at 25° C. for 2 hrs. LCMS showed the desired MS was given. The mixture was concentrated under vacuum to give a residue. Then the combined residue was diluted with DCM (5.00 L), adjusted to pH=8 with 2.5 M NaOH aqueous solution, and separated. The aqueous phase was extracted with DCM (3.00 L) again, then the aqueous solution was adjusted to pH=3 with 1 N HCl aqueous solution, then extracted with DCM (5.00 L*2), the combined organic layer was washed with brine (3.00 L), dried over Na₂SO₄, filtered and concentrated under vacuum. The crude was purified by column chromatography (SiO₂, DCM:MeOH=0:1-12:1, 0.1% HOAc, R_{f}=0.4). The residue was diluted with DCM (5.00 L), adjusted to pH=8 with 2.5 M NaOH aqueous solution, separated, the aqueous solution was extracted with DCM (3.00 L) again, then the aqueous solution was adjusted to pH=3 with 6 N HCl aqueous solution, extracted with DCM:MeOH=10:1 (5.00 L*2), the combined organic layer was washed with brine (2.00 L), dried over Na₂SO₄, filtered and concentrated under vacuum to give a residue. Then the residue was diluted with MeCN (5.00 L), concentrated under vacuum, repeat this procedure twice to remove water to give TRIS-PEG2-CBZ (1.25 kg, 1.91 mol, 78.1% yield, 95.8% purity) as light yellow oil. ¹HNMR: 400 MHz, MeOD, δ 7.30-7.35 (5H), 5.07 (s, 2H), 3.65-3.70 (m, 16H), 3.59 (s, 4H), 3.45 (t, J=5.6 Hz), 2.51 (t, J=6.0 Hz), 2.43 (t, 6.4 Hz).

### Scheme for the Preparation of TriNGal-TRIS-Peg2-Peg4-Phosph

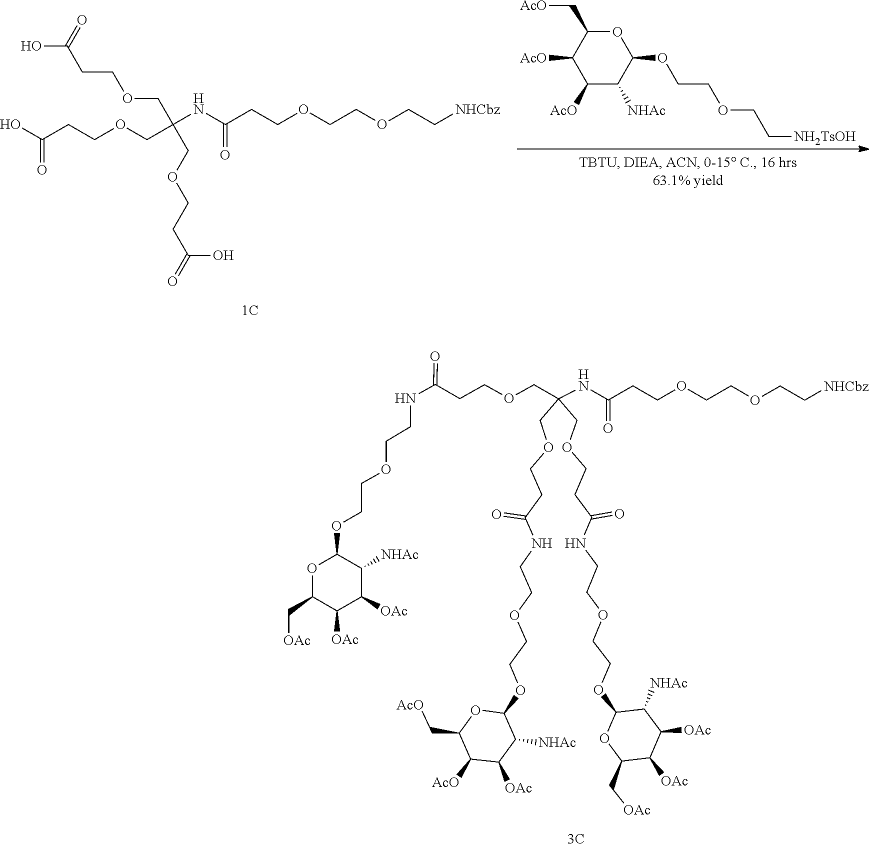

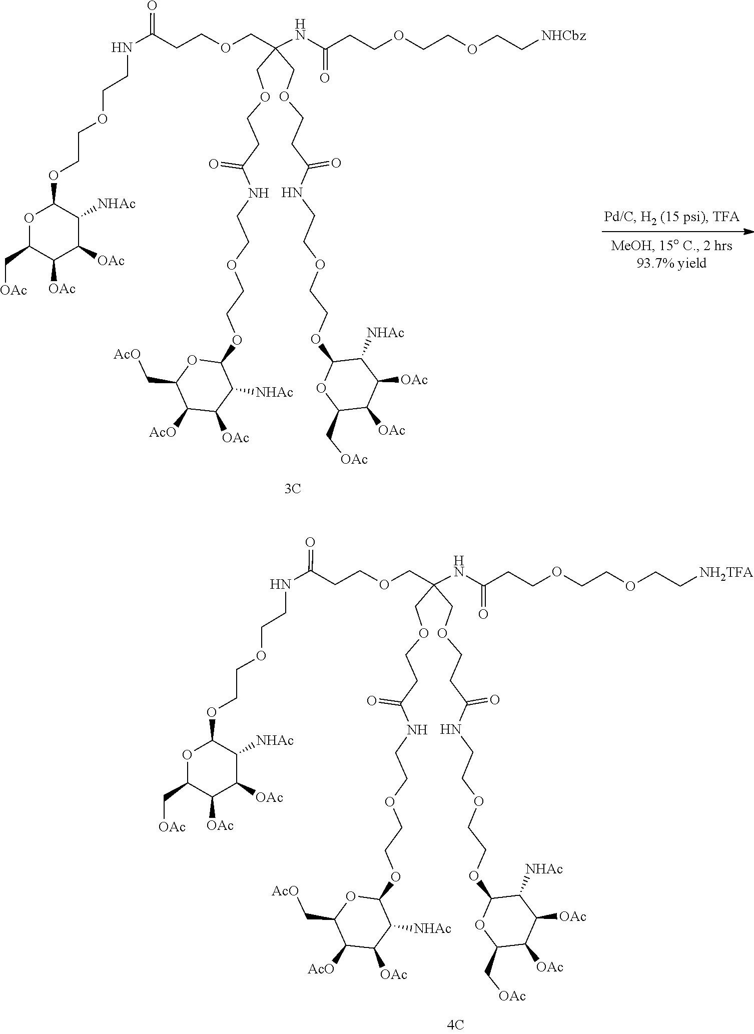

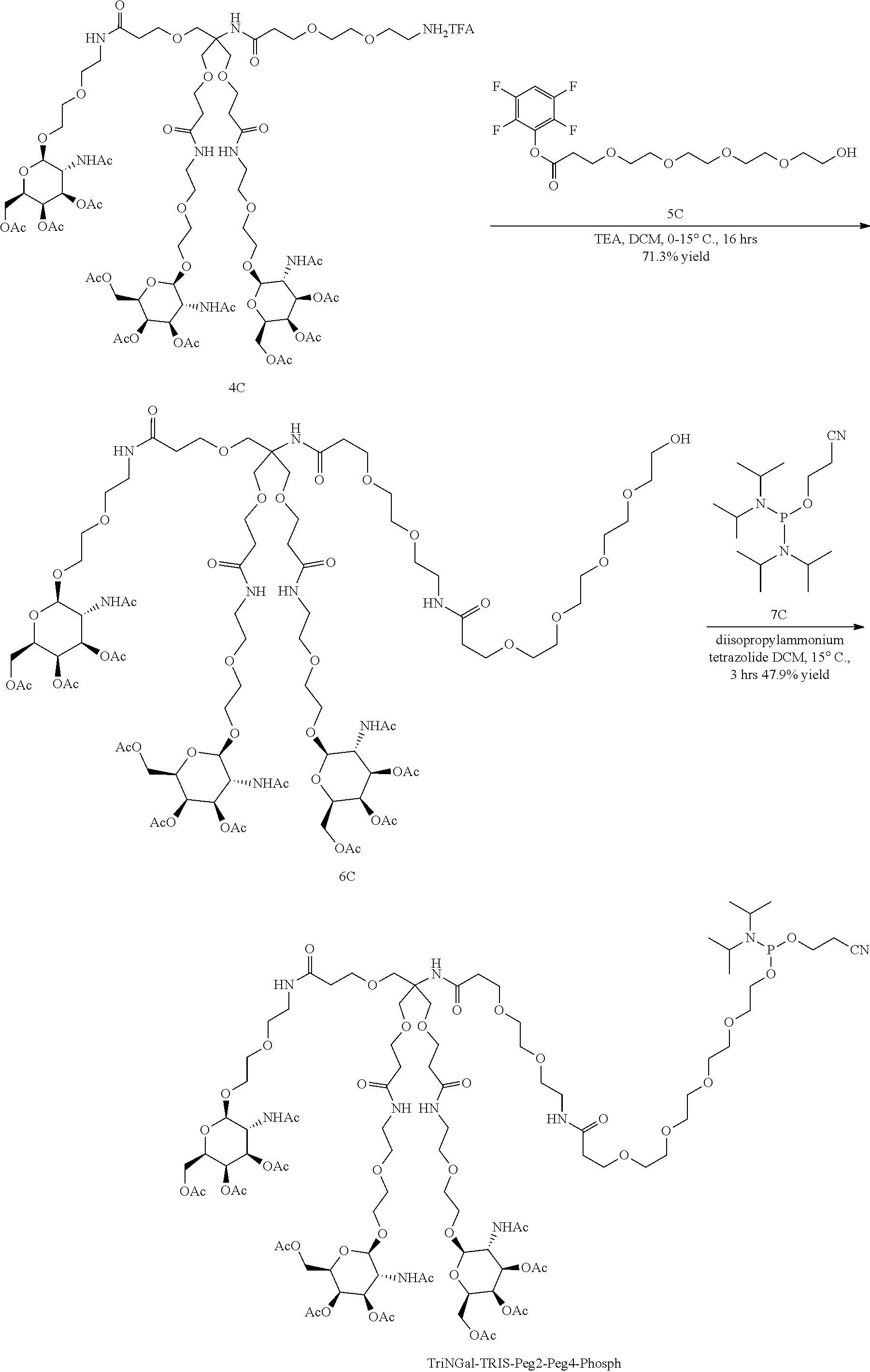

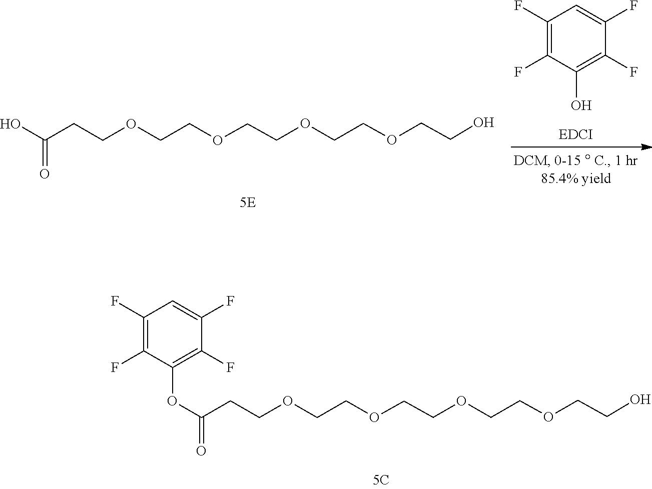 [SMILES: Oc1c(F)c(F)cc(F)c1F ; O=C(O)CCOCCOCCOCCOCCO ; O=C(CCOCCOCCOCCOCCO)Oc1c(F)c(F)cc(F)c1F]

### General Procedure for Preparation of Compound 3C

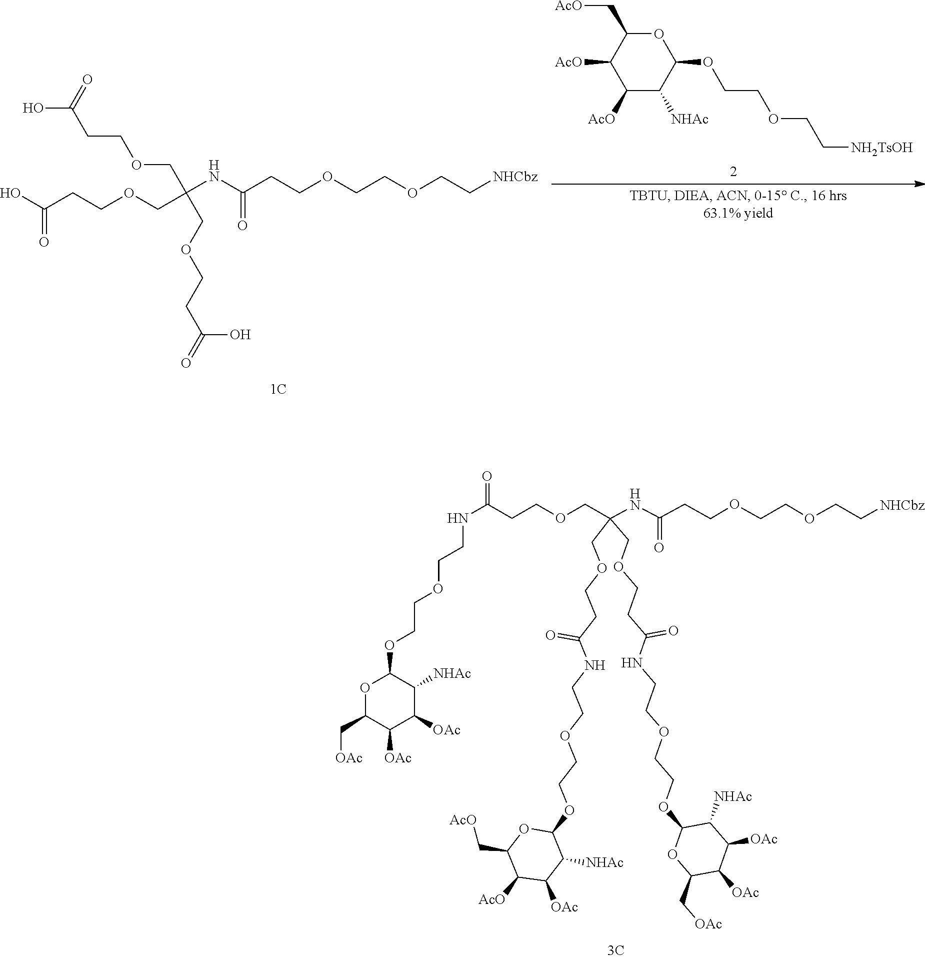

To a solution of Compound 1C (155 g, 245 mmol, 1.00 eq) in ACN (1500 mL) was added TBTU (260 g, 811 mmol, 3.30 eq), DIEA (209 g, 1.62 mol, 282 mL, 6.60 eq) and Compound 2C (492 g, 811 mmol, 3.30 eq, TsOH) at 0° C., the mixture was stirred at 15° C. for 16 hrs. LCMS showed the desired MS was given. The mixture was concentrated under vacuum to give a residue, then the mixture was diluted with DCM (2000 mL), washed with 1 N HCl aqueous solution (700 mL*2), then saturated NaHCO₃ aqueous solution (700 mL*2) and concentrated under vacuum. The crude was purified by column chromatography to give Compound 3C (304 g, 155 mmol, 63.1% yield, 96.0% purity) as a yellow solid.

### General Procedure for Preparation of Compound 4C

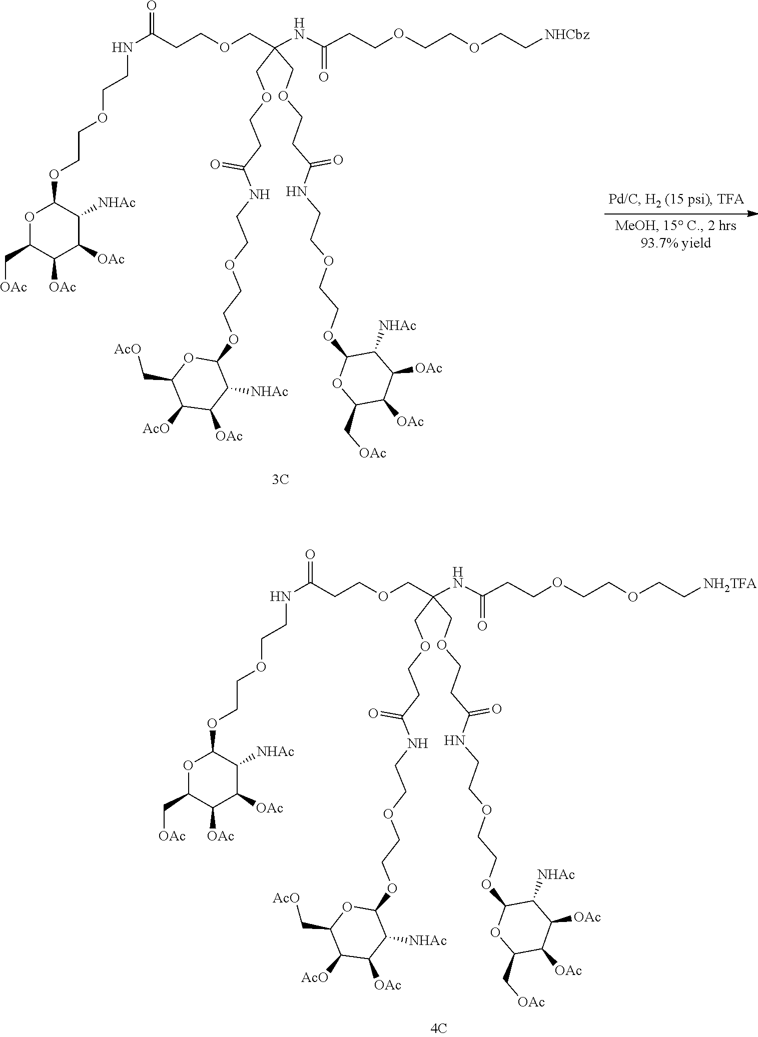

Two batches solution of Compound 3C (55.0 g, 29.2 mmol, 1.00 eq) in MeOH (1600 mL) was added Pd/C (6.60 g, 19.1 mmol, 10.0% purity) and TFA (3.34 g, 29.2 mmol, 2.17 mL, 1.00 eq), the mixture was degassed under vacuum and purged with H₂. The mixture was stirred under H₂ (15 psi) at 15° C. for 2 hours. LCMS showed the desired MS was given. The mixture was filtered and the filtrate was concentrated under vacuum to give Compound 4C (106 g, 54.8 mmol, 93.7% yield, 96.2% purity, TFA) as a white solid.

### General Procedure for Preparation of Compound 6C

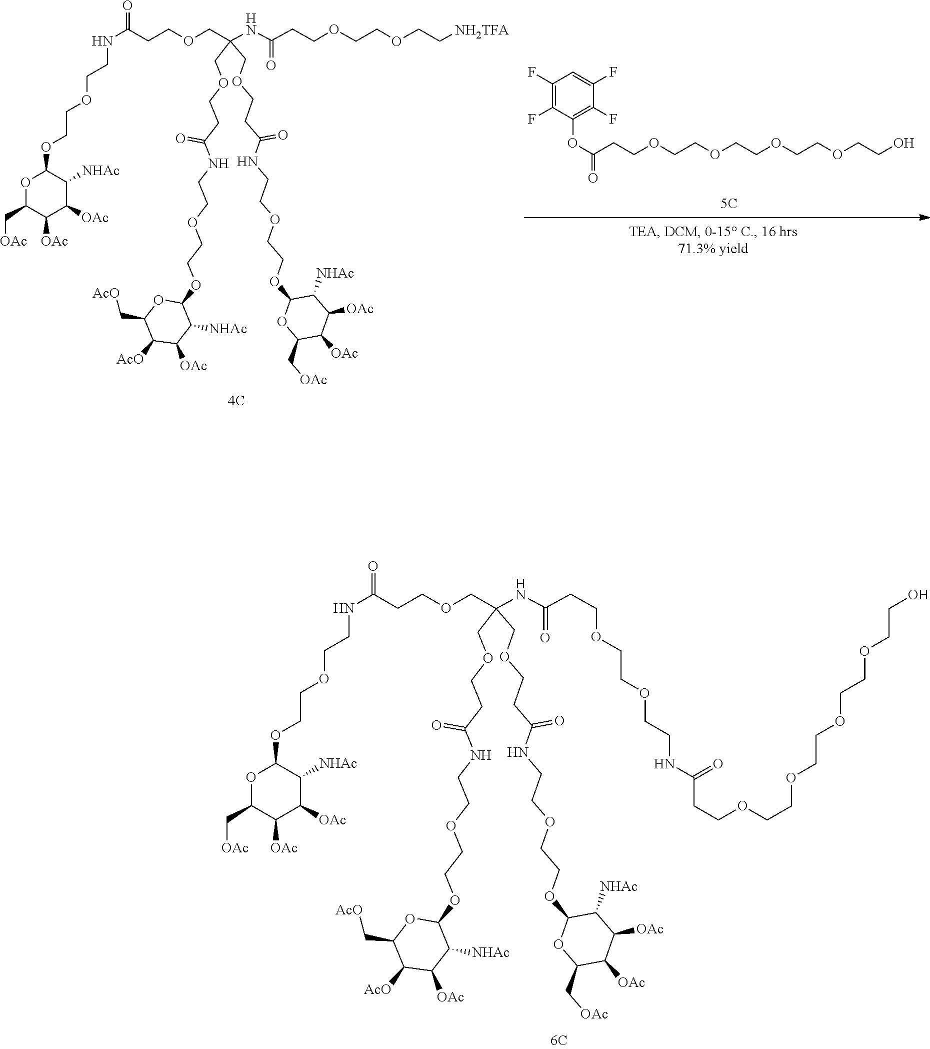

To a solution of Compound 4C (100 g, 53.7 mmol, 1.00 eq, TFA) and Compound 5C (23.3 g, 56.4 mmol, 1.05 eq) in DCM (1000 mL) was added TEA (21.7 g, 215 mmol, 29.9 mL, 4.00 eq) drop wise and stirred at 15° C. for 16 hrs. LCMS showed the desired was given. The mixture was diluted with DCM (500 mL), washed with sat Na₂CO₃ aqueous solution (1500 mL), then washed with brine (1000 mL*3), the organic layer was dried over Na₂SO₄, filtered and concentrated under vacuum to give Compound 6C (79.8 g, 38.3 mmol, 71.3% yield, ⁹5.8% purity) as a light yellow solid.

### General Procedure for Preparation of TriGNal-TRIS-Peg2-Peg4-Phosph

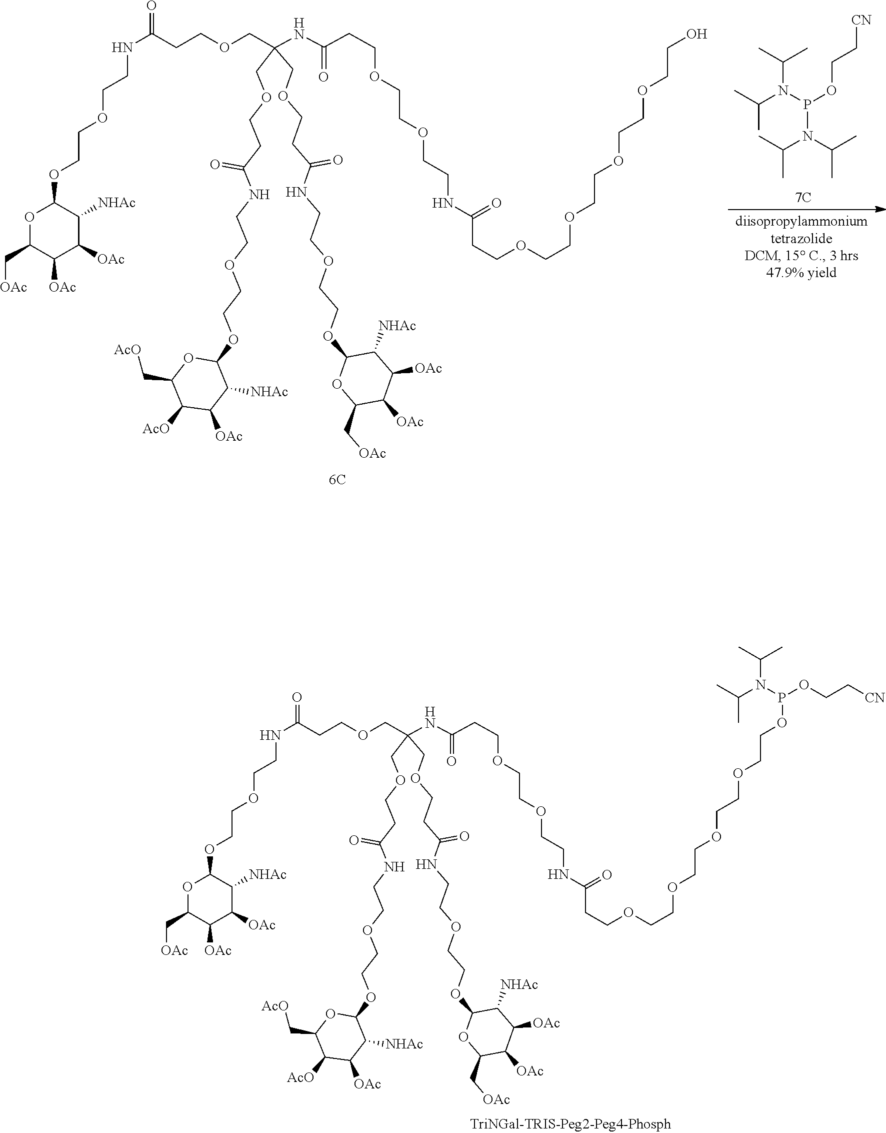

To a solution of Compound 6C (50.0 g, 23.8 mmol, 1.00 eq) in DCM (750 mL) was added diisopropylammonium tetrazolide (4.10 g, 23.8 mmol, 1.00 eq) and Compound 3C (7.18 g, 23.8 mmol, 7.57 mL, 1.00 eq) in DCM (10 mL) drop wise, the mixture was stirred at 15° C. for 1 h, then added Compound 3C (3.59 g, 11.9 mmol, 3.78 mL, 0.50 eq) in DCM (10 mL) drop wise, the mixture was stirred at 15° C. for 30 min, then added Compound 3C (3.59 g, 11.9 mmol, 3.78 mL, 0.50 eq) in DCM (10 mL) drop wise, the mixture was stirred at 15° C. for 1.5 hrs. The mixture was diluted with DCM (750 mL), washed with saturated NaHCO₃ aqueous solution (1000 mL*2), brine (1000 mL), the organic layer was dried overNa₂SO₄, filtered and concentrated under vacuum. The residue was purified with silica gel chromatography column to give TriNGal-TRIS-Peg2-Peg4-Phosph as a white solid (28.0 g, 16.29 mmol, 47.9% yield).

### General Procedure for Preparation of Compound 5C

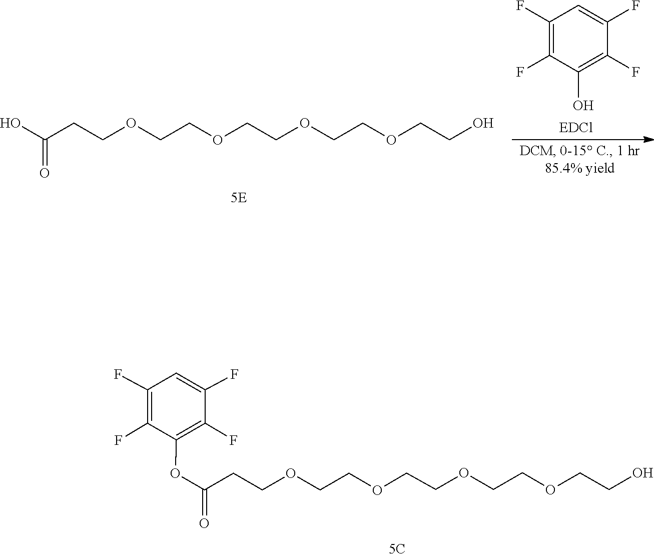 [SMILES: Oc1c(F)c(F)cc(F)c1F ; O=C(O)CCOCCOCCOCCOCCO ; O=C(CCOCCOCCOCCOCCO)Oc1c(F)c(F)cc(F)c1F]

To a solution of EDCI (70.5 g, 368 mmol, 1.00 eq) in DCM (300 mL) was added 2,3,5,6-tetrafluorophenol (61.1 g, 368 mmol, 1.00 eq) dropwise, then added Compound 5E (98.0 g, 368 mmol, 1.00 eq) in DCM (300 mL) at 0° C., then the mixture was stirred at 15° C. for 1 hr. The mixture was washed with saturated NaHCO₃ solution (100 mL) and concentrated under vacuum. The crude was purified by column chromatography to give Compound 5C (100 g, crude) as yellow oil.

### Example 7: In Vitro Hepatocyte Targeting Using GalNAc-Conjugated siRNAs and ASOs

In this experiment, a hepatocyte cell line expressing asialoglycoprotein receptors and GFP will be treated with anti-GFP siRNAs or ASOs conjugated to a GalNAc moiety compared to a control experiment where the hepatocyte cell line is treated with anti-GFP siRNAs or ASOs that are not conjugated to the GalNAc moiety. GFP mRNA and protein expression are measured, and the amount of GFP mRNA or protein expression in cells treated with the GalNAc-conjugated siRNAs or ASOs is normalized and compared to the amount of GFP mRNA or protein expression in cells treated with the siRNAs or ASOs that are not GalNAc-conjugated. This may allow for a determination of the hepatocyte targeting ability of the GalNAc moiety. Multiple GalNAc moieties may be conjugated to the siRNAs or ASOs and compared to see which GalNAc moiety results in optimal hepatocyte targeting. The GalNAc moieties to be tested in these experiments may a GalNAc moiety described herein such as Compound 1, Compound 2, or Compound 3.

Similar experiments may be performed in primary hepatocytes treated with the siRNAs or ASOs conjugated or not to a GalNAc moiety, and a target mRNA or target protein other than GFP may be assessed in the primary hepatocytes.

### Example 8: In Vivo Hepatocyte Targeting Using GalNAc-Conjugated siRNAs and ASOs

In this experiment, siRNAs or ASOs targeting a target mRNA will be conjugated to a GalNAc moiety and administered to mice (n=5/group), and compared to a control experiment where the mice are administered siRNAs or ASOs without GalNAc conjugation. Mice are sacrificed 2 days later, and livers are frozen, later homogenized, and tested for target mRNA and protein expression. The amount of target mRNA or protein expression in the livers of mice treated with the GalNAc-conjugated siRNAs or ASOs is normalized and compared to the amount of GFP mRNA or protein expression in the livers of mice treated with the siRNAs or ASOs that are not GalNAc-conjugated. This may allow for a determination of the liver targeting ability of the GalNAc moiety. Multiple GalNAc moieties may be conjugated to the siRNAs or ASOs and compared to see which GalNAc moiety results in optimal liver targeting. The GalNAc moieties may be those that exhibit the greatest degree of hepatocyte targeting in Example 6. The GalNAc moieties to be tested in these experiments may a GalNAc moiety described herein such as Compound 1, Compound 2, or Compound 3.

### Example 9: Inhibition of a Target mRNA in a Mouse Model of a Liver Disease Using GalNAc-Conjugated siRNAs and ASOs

In this experiment, a murine model of a liver disease (in this case, fatty liver disease) will be used to evaluate the effect of siRNA or ASO inhibition of a target mRNA. The target mRNA may encode any target protein where overexpression or overactivation plays a pathological role in the liver disease. In the murine model, fatty liver disease is induced by feeding mice a Western Diet (WD) containing 21.1% fat, 41% Sucrose, and 1.25% Cholesterol by weight (Teklad diets, TD. 120528) and a high sugar solution (23.1 g/L d-fructose (Sigma-Aldrich, G8270) and 18.9 g/L d-glucose (Sigma-Aldrich, F0127)) for 12 weeks. At 4-week-old C57BL/6J mice are fed a Western Diet instead of regular chow for 12 weeks. The GalNAc moieties to be used in this experiments may a GalNAc moiety described herein such as Compound 1, Compound 2, or Compound 3.

Briefly, mice are divided into five groups: Group 1: a fatty liver disease group treated with non-targeting control siRNA, Group 2: a fatty liver disease group treated with non-targeting control ASO, Group 3: a fatty liver disease group treated with an siRNA targeting a target mRNA, Group 4: a fatty liver disease group treated with an ASO targeting a target mRNA, Group 5: control mice on a normal chow diet. Each group contains eight mice (4 males, 4 females). The siRNAs and ASOs of Groups 1-4 each include a GalNAc moiety attached to the siRNA or ASO (for example, a GalNAc moiety of Compound 1, Compound 2, or Compound 3).

At weeks 12 weeks of Western Diet, blood samples are collected from each group prior first treatment.

Administration of siRNA or ASO is achieved with a 200 μL subcutaneous injection of naked siRNA or ASO resuspended in PBS at concentration of 10 μM. On Study Day 0, Group 1 mice will be injected subcutaneously with non-targeting control siRNA, Group 2 mice will be injected subcutaneously with non-targeting control ASO, Group 3 mice will be injected subcutaneously with siRNA1 targeting the target mRNA in a mouse, Group 4 mice will be injected subcutaneously with ASO1 targeting the target mRNA in a mouse, and Group 5 mice will be injected subcutaneously with vehicle. Every other week thereafter starting on Day 14 the animals from each group will be dosed as on Day 0 for a total of 5 injections.

Weekly blood draws will be taken and serum and plasma isolated. Serum ALT, AST, total cholesterol and triglyceride levels are measured using VITROS 5,1 FS (Ortho Clinical Diagnostics). Non-fasting plasma insulin is measured with the Ultrasensitive Mouse Insulin ELISA kit (Crystal Chem, 90080) according to the manufacturer's instructions. Non-fasting blood glucose is assayed with the One Touch Ultra (Life Scan). HOMA IR and QUICKI are calculated.

At the end of 12 weeks of Western Diet and siRNA/ASO treatment, mice are sacrificed by cervical dislocation following an intraperitoneal injection of 0.3 ml Nembutal (5 mg/ml). Terminal serum draw is collected via cardiac puncture and final serum ALT, AST, total cholesterol and triglyceride levels are measure along with non-fasting plasma insulin and glucose. Livers are removed and divided into three sections; one section placed in RNA later for mRNA isolation, one section flash-frozen for protein isolation, one section fixed in formalin and then paraffin-embedded.

mRNA is isolated from tissue placed in RNA later solution using the PureLink kit according to the manufacturer's protocol (ThermoFisher Cat. No. 12183020). The reverse transcriptase reaction is performed according to the manufacturer's protocol. Samples are stored at −80° C. until real-time qPCR is performed in triplicate using TaqMan Gene Expression Assays (Applied Biosystems FAM-probes using a BioRad iCycler). A decrease in target mRNA expression in the liver tissue from mice is dosed with the siRNAs and ASOs compared to target mRNA levels in the liver tissue from mice is dosed with the non-specific control siRNA and ASO. There is an expected decrease in the amount of SDF-1 in the liver tissue from mice that receive the siRNAs and ASOs compared to the amount of SDF-1 in the liver tissue from mice that receive the non-specific control siRNA or ASO. These results show that the siRNAs and ASOs elicit knockdown of the target mRNA and target protein in liver tissue, and that the decrease in target mRNA and target protein expression is correlated with a decrease in SDF-1 production.

Formalin-fixed, paraffin-embedded liver sections are stained with hematoxylin and eosin (H&E) for assessment of liver histology, with Sirius Red (Sigma, 365548-5G)/Fast Green (Sigma, F258) for assessment of fibrosis, and with periodic acid-Schiff (PAS) for assessment of glycogen accumulation. NAFLD Activity Score (NAS) and fibrosis stage are evaluated by an expert pathologist according to the NASH CRN scoring system 13. The histological scoring is performed blinded, with no knowledge by the pathologist of the treatment(s) received. These results show that the siRNAs and ASOs elicit knockdown of the target mRNA and target protein in liver tissue, and that the decrease in expression of the target mRNA and target protein is correlated with a decrease in NAS and NASH CRN.

### Example 10: Inhibition of a Mouse Model of a Liver Disease

In this experiment, a murine model of a liver disease (in this case, hypertriglyceridemia) will be used to evaluate the effect of siRNA or ASO inhibition of a target protein expressed in the liver compared to an anti-mouse target protein antibody. The mouse strain C57Bl/6 Apoetm1Unc mice will be maintained on a high fat Western diet (Research Diets, D12492; 60% fat by calories). The target protein may be any target protein where overexpression or overactivation plays a pathological role in the liver disease. The GalNAc moieties to be used in this experiments may a GalNAc moiety described herein such as Compound 1, Compound 2, or Compound 3.

Four groups of mice (n=16/group) will be utilized in this study. Animals will be maintained on a high fat diet during the study. On Day −4 before the first injection, chow will be removed for an overnight fast. On Day −3 before the first injection, all animals will be anesthetized and 300 μL of blood collected in serum separator tubes via the submandibular vein to assess baseline triglyceride, serum glucose, insulin sensitivity, total cholesterol levels, HDL Cholesterol levels, liver function and serum levels of target protein. On Study Day 0, Group 1 mice will be injected intraperitoneally with 600 μL normal saline, Group 2 mice will be injected intraperitoneally with 600 μg of anti-mouse target protein antibody in 600 μL, Group 3 mice will be injected subcutaneously with 150 μg of GalNAc-siRNA targeting an mRNA encoding the target protein in a mouse in 200 μL of normal saline, and Group 4 mice will be injected subcutaneously with 150 μg of GalNAc-ASO targeting the mRNA encoding the target protein in 200 μL of normal saline. On the afternoon of Day 3, the chow will be removed from all Groups for an overnight fast. On Day 4, the animals from all Groups will be anesthetized and 150 μL of blood collected in serum separator tubes via the submandibular vein to assess serum triglycerides, glucose, total cholesterol, HDL cholesterol and levels of the target protein. Animals from all groups will then undergo an oral glucose tolerance test and insulin tolerance test to evaluate insulin sensitivity. Chow will be supplied again as normal after blood has been collected and insulin sensitivity tests conducted. Weekly thereafter starting on Day 7 the animals from Group 2 will be dosed as on Day 0 for a total of 15 injections. Every other week thereafter starting on Day 14 the animals from Group 3 and Group 4 will be dosed as on Day 0 for a total of 8 injections. Every other week starting on Day 10, the mice from all Groups will be fasted (overnight) and bled (150 μL into serum separator tubes) to assess serum triglyceride, glucose, total cholesterol, HDL cholesterol and levels of target protein, and undergo insulin sensitivity tests. On the third day after the final injection, the chow will be removed from all Groups for an overnight fast. On the fourth day after the final injection, the animals from all Groups will be anesthetized, euthanized and bled via cardiac puncture to collect 500 μL of blood into serum separator tubes to assess triglyceride, serum glucose, insulin sensitivity, total cholesterol levels, HDL cholesterol levels, liver function and serum levels of target protein. Tissue from the liver, small intestine and mesenteric lymph nodes will be collected from all animals and immersed in 10% neutral buffered formalin for histopathological analysis. A liver sample will also be collected from all animals and placed in RNAlater. The levels of target mRNA will be assessed by RT-qPCR using TaqMan assays for the mouse target protein and the mouse housekeeping gene PPIA.

Animals treated with the antibody (Group 2), mice treated with the GalNAc-siRNA (Group 3), and mice treated with the GalNAc-ASO (Group 4) are expected to have decreased triglycerides, total serum cholesterol, serum glucose as well as decreased serum target protein levels, and increased HDL cholesterol and insulin sensitivity, compared with mice from Group 1 (saline). Animals in Group 2 and Group 3 are also expected to have decreased target mRNA in liver samples.

### Example 11: Inhibition of a Target mRNA in Non-Human Primates Using GalNAc-siRNA and GalNAc-ASO

In this experiment, a NHP model of hypertriglyceridemia is used to evaluate the effect of siRNA or ASO inhibition of the target mRNA expressed in the liver. The target protein may be any target protein where overexpression or overactivation plays a pathological role in the liver disease. Three groups of cynomolgus monkeys will be used (n=5/group) that are placed on a high-fat diet (Western Primate Diet, 5S2T) before the initiation of the study. Alternatively, three groups of rhesus monkeys will be used (n=5/group) that are placed on a high fructose diet before the initiation of the study. Animals are to be given 7 biweekly subcutaneous injections of saline (Group 1), GalNAc-siRNA (Group 2), or GalNAc-ASO (Group 3). The modified GalNAc-siRNA sequences may include any modification pattern described herein. The GalNAc moieties to be used in this experiments may a GalNAc moiety described herein such as Compound 1, Compound 2, or Compound 3. Blood samples for lipid and glycemic measurements will be collected at baseline and at 4, 8, and 14 weeks of the study and analyzed for lipid content, serum glucose, insulin sensitivity and target protein. All animals from each group are necropsied 2 weeks after the last blood collection. Tissue from the liver, small intestine and mesenteric lymph nodes will be collected from all animals and immersed in 10% neutral buffered formalin for histopathological analysis. A liver sample will also be collected from all animals and placed in RNAlater. The levels of target mRNA will be assessed by RT-qPCR using TaqMan assays for cynomolgus or rhesus target protein and the cynomolgus or rhesus housekeeping gene PPIA.

It is expected that animals treated with the GalNAc-siRNA (Group 2) and animals treated with the GalNAc-ASO (Group 3) will show decreased triglycerides, total serum cholesterol and serum glucose as well as decreased serum target protein levels, and increased HDL cholesterol and insulin sensitivity, compared with animals from Group 1 (saline). It is also expected that animals in Group 1 and Group 3 will show decreased target mRNA in liver samples.

### Example 12: Inhibition of a Target mRNA in a Clinical Trial Using GalNAc-siRNA and GalNAc-ASO

In this study, human subjects with hypertriglyceridemia are used to evaluate the effect of siRNA or ASO inhibition of a target mRNA expressed in the liver. The target protein may be any target protein where overexpression or overactivation plays a pathological role in the liver disease. Selection criteria for inclusion in the study are ages 40-90, BMI≥30, and serum triglycerides ≥250 mg/dL. Three groups of subjects will be included (n=15/group) in the study. Subjects are to be given 5 weekly subcutaneous injections of saline (Group 1), GalNAc-siRNA (Group 2), or GalNAc-ASO (Group 3). The GalNAc moieties to be used in these experiments may include a GalNAc moiety described herein such as Compound 1, Compound 2, or Compound 3

The siRNA or ASO sequences are to be from a selection set that shows high activity in cells in culture or in experiments describe in the other examples. Blood samples for lipid and glycemic measurements will be collected at baseline and at 3, 6, and 12 weeks of the study and analyzed for lipid content, serum glucose, insulin sensitivity, target protein, and liver and kidney function.

It is expected that subjects treated with the GalNAc-siRNA (Group 2) and subjects treated with GalNAc-ASO (Group 3) will show decreased triglycerides, total serum cholesterol and serum glucose as well as decreased serum target protein levels, and increased HDL cholesterol and insulin sensitivity, compared with subjects from Group 1 (saline).

### Example 13: Oligonucleotide Synthesis

RNAi agents (e.g. siRNAs) were synthesized according to phosphoramidite technology on a solid phase used in oligonucleotide synthesis. A K&A oligonucleotide synthesizer was used. Syntheses were performed on a solid support made of controlled pore glass (CPG, 500 Å or 600 Å, obtained from AM Chemicals, Oceanside, CA, USA). All 2′-OMe and 2′-F phosphoramidites were purchased from Hongene Biotech (Union City, CA, USA). All phosphoramidites were dissolved in anhydrous acetonitrile (100 mM) and molecular sieves (3 Å) were added. 5-Benzylthio-1H-tetrazole (BTT, 250 mMin acetonitrile) or 5-Ethylthio-1H-tetrazole (ETT, 250 mM in acetonitrile) was used as activator solution. Coupling times were 9-18 min (EmpGalNAc), 6 min (2′OMe and 2′F). In order to introduce phosphorothioate linkages, a 100 mM solution of 3-phenyl 1,2,4-dithiazoline-5-one (POS, obtained from PolyOrg, Inc., Leominster, Mass., USA) in anhydrous acetonitrile was employed.

After solid phase synthesis, the dried solid support was treated with a 1 volume solution of 40 wt. % methylamine in water and 28% ammonium hydroxide solution (Aldrich) for two hours at 30° C. The solution was evaporated and the solid residue was reconstituted in water and purified by anionic exchange HPLC using a TKSgel SuperQ-5PW 13u column. Buffer A was 20 mM Tris, 5 mM EDT A, pH 90 an d contained 20% AcetonitrlHe and buffer B was the same as buffer A with the addition of 1 M sodium chloride. UV traces at 260 nm were recorded. Appropriate fractions were pooled then desalted using Sephadex G-25 medium.

Equimolar amounts of sense and antisense strand were combined to prepare a duplex. The duplex solution was prepared in 0.1×PBS (Phosphate-Buffered Saline 1×, Gibco). The duplex solution was annealed at 95° C. for 5 min, and cooled to room temperature slowly. Duplex concentration was determined by measuring the solution absorbance on a UV-Vis spectrometer at 260 nm in 0.1×PBS For some experiments, a conversion factor was calculated from an experimentally determined extinction coefficient.

### Example 14: Determining the Activity of EMPGalNAc Targeted ANGPTL4 1176 and 1177

The following oligonucleotides in Table 2 were synthesized to determine activity targeted to a target gene comprising Angiopoietin-like 4 (ANGPTL4). ETD01176 included modification pattern 2S #2 and 3AS. ETD01177 and ETD0971 included modification patterns 5 S and 3AS.

TABLE 2

| | Sense strand sequence | SEQ ID | Antisense strand | SEQ ID |
|---|---|---|---|---|
| siRNA Name | (5′ to 3′) | NO: | sequence (5′ to 3′) | NO: |
| Unmodified | GGACAAUACUUCCAC | 23 | UAGAGUGGAAGUA | 27 |
| ETD01176 | UCUA | | UUGUCC | |
| Unmodified | GGACAAUACUUCCAC | 23 | UAGAGUGGAAGUA | 27 |
| ETD01177 | UCUA | | UUGUCC | |
| Unmodified | GGACAAUACUUCCAC | 23 | UAGAGUGGAAGUA | 27 |
| ETD0971 | UCUA | | UUGUCC | |
| ETD01176 | [EMPGalNAc]ggacAfaUf | 24 | usAfsgagUfggaaguaUf | 28 |
| | AfCfuuccacucuasusu | | uGfuccsusu | |
| ETD01177 | [EMPGalNAc]gsgsacAfa | 25 | usAfsgagUfggaaguaUf | 28 |
| | UfAfCfuuccacucuasusu | | uGfuccsusu | |
| ETD0971 | gsgsacAfaUfAfCfuuccacu | 26 | usAfsgagUfggaaguaUf | 28 |
| | cuasusu[3′ GalNAc] | | uGfuccsusu | |

EMPGalNAc had the following structure:

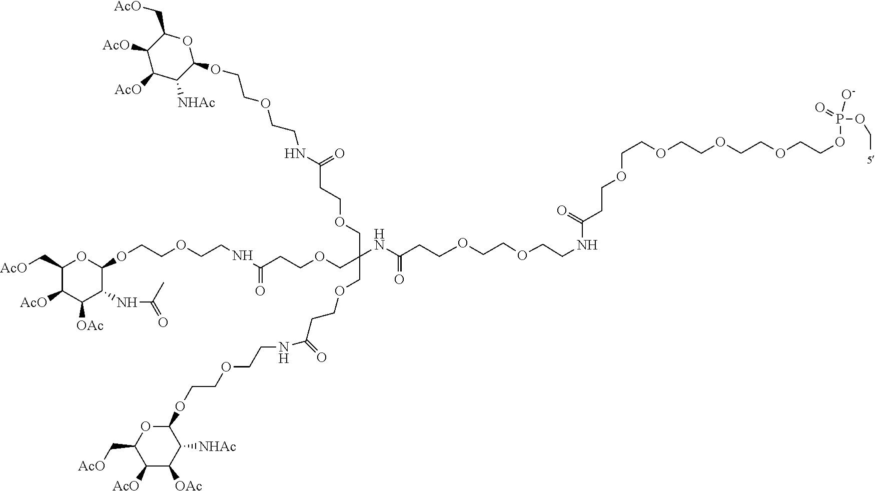 [SMILES: CCOP(=O)([O-])OCCOCCOCCOCCOCCC(=O)NCCOCCOCCC(=O)NC(COCCC(=O)NCCOCCO[C@@H]1O[C@H](COC(C)=O)[C@H](OC(C)=O)[C@H](OC(C)=O)[C@H]1NC(C)=O)(COCCC(=O)NCCOCCO[C@@H]1O[C@H](COC(C)=O)[C@H](OC(C)=O)[C@H](OC(C)=O)[C@H]1NC(C)=O)COCCC(=O)NCCOCCO[C@@H]1O[C@H](COC(C)=O)[C@H](OC(C)=O)[C@H](OC(C)=O)[C@H]1NC(C)=O]

3′ GalNAc had the following structure:

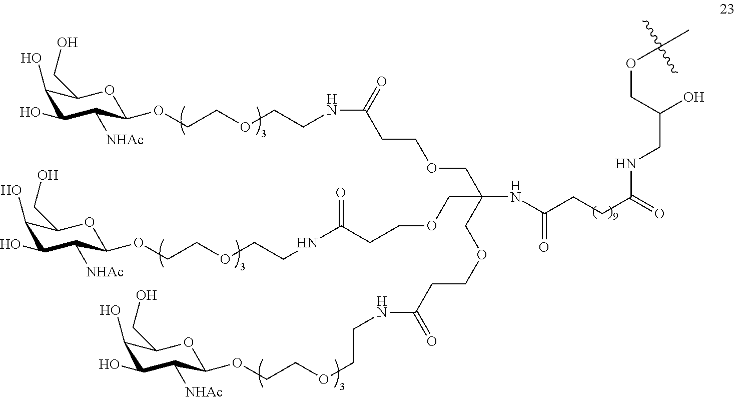

Three groups (n=3/group) of male ICR mice (Harlan) were utilized in this study. On Study Day 0, Group 1 mice were injected subcutaneously with 100 μL of sterile PBS, Group 2 mice were subcutaneously injected with 100 μg of ETD0971 in 100 μL of sterile PBS, Group 3 mice were subcutaneously injected with 100 μg of ETD01176 in 100 μL of sterile PBS, and group 4 mice were subcutaneously injected with 100 μg of ETD01177 in 100 μL of sterile PBS. On Day 14 the animals from all Groups were euthanized. A liver sample was collected from all animals and placed in RNAlater™ Stabilization Solution (Thermo Fisher, Catalog #AM7020). The liver samples were processed in homogenization buffer (Maxwell RSC simplyRNA Tissue Kit) using Soft Tissue Homogenizing Kit CK14 (Bertin Instruments), Catalog #P000933-LYSKO-A) in a Percellys 24 tissue homogenizer (Bertin Instruments) set at 5000 rpm for two 10 second cycles. Total RNA from the liver lysate was purified on a Maxwell RSC 48 platform (Promega Corporation) according to the manufacturer's recommendations. The relative level of ENTPD8 mRNA in each liver sample was assessed by RT-qPCR on a QuantStudio 6 Pro instrument (Applied Biosystems) using TaqMan assays for mouse ANGPTL4 (ThermoFisher, assay #Mm00480431_m1) and the mouse housekeeping gene PPIA (ThermoFisher, assay #Mm02342430_g1), and then normalized to the mean value from the control mice (Group 1) using the delta-delta Ct method.

The results of the liver mRNA analyses are shown in Table 3. Animals treated ETD0971 (Group 2), ETD01176 (Group 3) or ETD01177 (group 4) showed decreased liver ANGPTL4 mRNA levels compared with mice injected with PBS (Group 1) with EMPGalNAc targeted-ETD01177 achieving higher levels of knockdown than its 3′ GalNAc comparator ETDO0971. Therefore, a GalNAc of Formula (I) such as EMPGalNAc or other may be useful for targeting oligonucleotides such as siRNAs to hepatocytes or liver.

TABLE 3
ANGPTL4 mRNA liver levels in mice treated with ETD0971,
ETD01176 or ETD01177

| | | | Individual Relative | Mean Relative Liver |
|---|---|---|---|---|
| | | | Liver ANGPTL4 | ANGPTL4 mRNA |
| Group # | Treatment | Mouse # | mRNA Level | Level |
| 1 | PBS | 1 | 1.074 | 1.00 |
| | | 2 | 0892 | |
| | | 3 | 1.045 | |
| 2 | ETD00971 | 4 | 047 | 0.38 |
| | | 5 | 0.31 | |
| | | 6 | 0.36 | |
| 3 | ETD01176 | 7 | 0.79 | 0.57 |
| | | 8 | 0.45 | |
| | | 9 | 0.46 | |
| 4 | ETD01177 | 7 | 0.10 | 0.19 |
| | | 8 | 0.18 | |
| | | 9 | 0.28 | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

What is claimed is:

1. A compound represented by Formula (I): 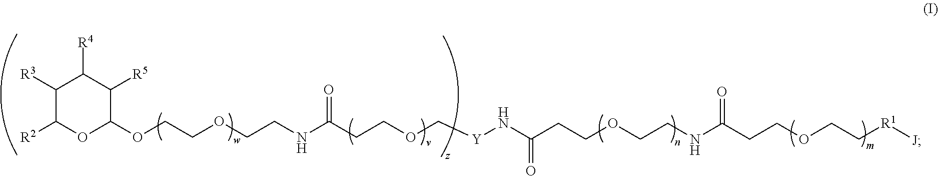
or a salt thereof, wherein
J is an oligonucleotide;
each w is independently selected from any value from 1 to 20;
each v is independently selected from any value from 1 to 20;
n is selected from any value from 1 to 20;
m is selected from any value from 1 to 20;
z is selected from any value from 1 to 3, wherein
if z is 3, Y is C
if z is 2, Y is CR⁶, or
if z is 1, Y is C(R⁶)₂;
R¹ is a linker selected from:
—O—, —S—, —N(R⁷)—, —C(O)—, —C(O)N(R⁷)—, —N(R⁷)C(O)—, —N(R⁷)C(O)N(R⁷)—, —OC(O)N(R⁷)—, —N(R⁷)C(O)O—, —C(O)O—, —OC(O)—, —S(O)—, —S(O)₂—, —OS(O)₂—, —OP(O)(OR⁷)O—, —SP(O)(OR⁷)O—, —OP(S)(OR⁷)O—, —OP(O)(SR⁷)O—, —OP(O)(OR⁷)S—, —OP(O)(O⁻)O—, —SP(O)(O⁻)O—, —OP(S)(O⁻)O—, —OP(O)(S⁻)O—, —OP(O)(O⁻)S—, —OP(O)(OR⁷)NR⁷—, —OP(O)(N(R⁷)₂)NR⁷—, —OP(OR⁷)O—, —OP(N(R⁷)₂)O—, —OP(OR⁷)N(R⁷)—, and —OPN(R⁷)₂NR⁷—;
each R² is independently selected from:
C₁₋₆ alkyl optionally substituted with one or more substituents independently selected from halogen, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
R³ and R⁴ are each independently selected from:
—OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁵ is independently selected from:
—OC(O)R⁷, —OC(O)N(R⁷)₂, —N(R⁷)C(O)R⁷, —N(R⁷)C(O)N(R⁷)₂, —N(R⁷)C(O)OR⁷, —C(O)R⁷, —C(O)OR⁷, and —C(O)N(R⁷)₂;
each R⁶ is independently selected from:
hydrogen;
halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷; and
C₁₋₆ alkyl optionally substituted with one or more substituents independently selected from halogen, —CN, —OR⁷, —SR⁷, —N(R⁷)₂, —C(O)R⁷, —C(O)N(R⁷)₂, —N(R⁷)C(O)R⁷, —N(R⁷)C(O)N(R⁷)₂, —OC(O)N(R⁷)₂, —N(R⁷)C(O)OR⁷, —C(O)OR⁷, —OC(O)R⁷, and —S(O)R⁷;
each R⁷ is independently selected from:
hydrogen;
C₁₋₆ alkyl, C₂₋₆ alkenyl, and C₂₋₆ alkynyl, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, and 3- to 10-membered heterocycle; and
C₃₋₁₀ carbocycle, and 3- to 10-membered heterocycle, each of which is optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₁₋₆ alkyl, C₂₋₆ alkenyl, C₂₋₆ alkynyl, C₃₋₁₀ carbocycle, 3- to 10-membered heterocycle, and C₁₋₆haloalkyl.

2. The compound or salt of claim 1, wherein each w is independently selected from any value from 1 to 10.

3. The compound or salt of claim 1, wherein each w is independently selected from any value from 1 to 5.

4. The compound or salt of claim 1, wherein each w is 1.

5. The compound or salt of claim 1, wherein each v is independently selected from any value from 1 to 10.

6. The compound or salt of claim 1, wherein each v is independently selected from any value from 1 to 5.

7. The compound or salt of claim 1, wherein each v is 1.

8. The compound or salt of claim 1, wherein n is selected from any value from 1 to 10.

9. The compound or salt of claim 1, wherein n is selected from any value from 1 to 5.

10. The compound or salt of claim 1, wherein n is 2.

11. The compound or salt of claim 1, wherein m is selected from any value from 1 to 10.

12. The compound or salt of claim 1, wherein m is selected from any value from 1 to 5.

13. The compound or salt of claim 1, wherein m is 4.

14. The compound or salt of claim 1, wherein z is 3 and Y is C.

15. The compound or salt of claim 1, wherein R¹ is —OP(O)(OR⁷)O—.

16. The compound or salt of claim 1, wherein R² is selected from C₁₋₃ alkyl substituted with one or more substituents independently selected from halogen, —OR⁷, —OC(O)R⁷, —SR⁷, —N(R⁷)₂, —C(O)R⁷, and —S(O)R⁷.

17. The compound or salt of claim 1, wherein R² is selected from C₁₋₃ alkyl substituted with one or more substituents independently selected from —OR⁷, —OC(O)R⁷, —SR⁷, and —N(R⁷)₂.

18. The compound or salt of claim 1, wherein R³ is selected from halogen, —OR⁷, —SR⁷, —N(R⁷)₂, —C(O)R⁷, —OC(O)R⁷, and —S(O)R⁷.

19. The compound or salt of claim 1, wherein R³ is selected from —OR⁷, —SR⁷, —OC(O)R⁷, and —N(R⁷)₂.

20. The compound or salt of claim 1, wherein R⁴ is selected from halogen, —OR⁷, —SR⁷, —N(R⁷)₂, —C(O)R⁷, —OC(O)R⁷, and —S(O)R⁷.

21. The compound or salt of claim 1, wherein R⁴ is selected from —OR⁷, —SR⁷, —OC(O)R⁷, and —N(R⁷)₂.

22. The compound or salt of claim 1, wherein R⁵ is selected from —OC(O)R⁷, —OC(O)N(R⁷)₂, —N(R⁷)C(O)R⁷, —N(R⁷)C(O)N(R⁷)₂, and —N(R⁷)C(O)OR⁷.

23. The compound or salt of claim 1, wherein R⁵ is selected from —OC(O)R⁷ and —N(R⁷)C(O)R⁷.

24. The compound or salt of claim 1, wherein each R⁷ is independently selected from:
hydrogen; and
C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, —NH(C₁₋₆ alkyl), C₃₋₁₀ carbocycle, or 3- to 10-membered heterocycle.

25. The compound or salt of claim 1, wherein each R⁷ is independently selected from C₁₋₆ alkyl optionally substituted with one or more substituents independently selected from halogen, —CN, —OH, —SH, —NO₂, —NH₂, ═O, ═S, —O—C₁₋₆ alkyl, —S—C₁₋₆ alkyl, —N(C₁₋₆ alkyl)₂, and —NH(C₁₋₆ alkyl).

26. The compound of claim 1, wherein the compound comprises:

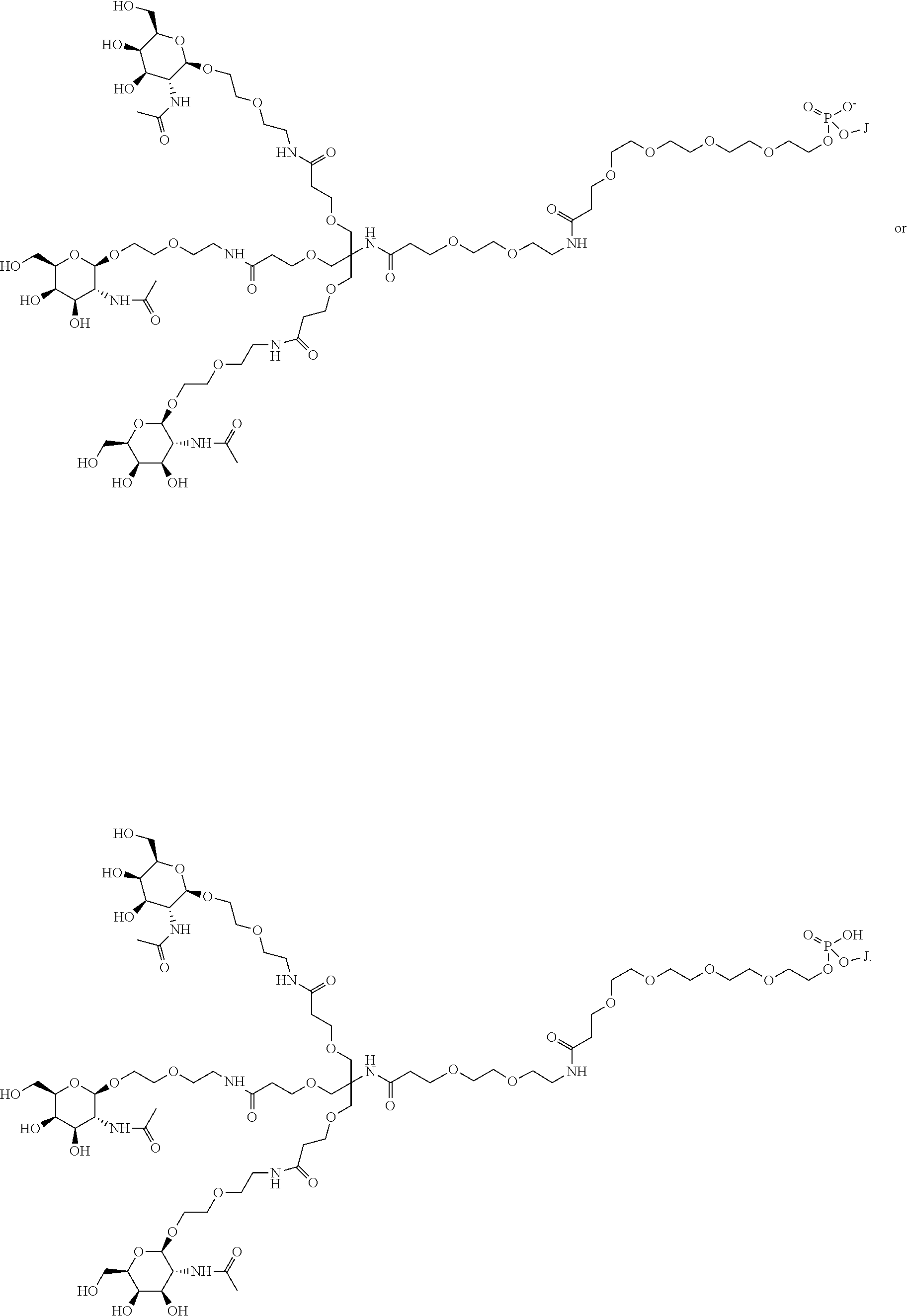

27. The compound or salt of claim 1 represented by Formula (II): 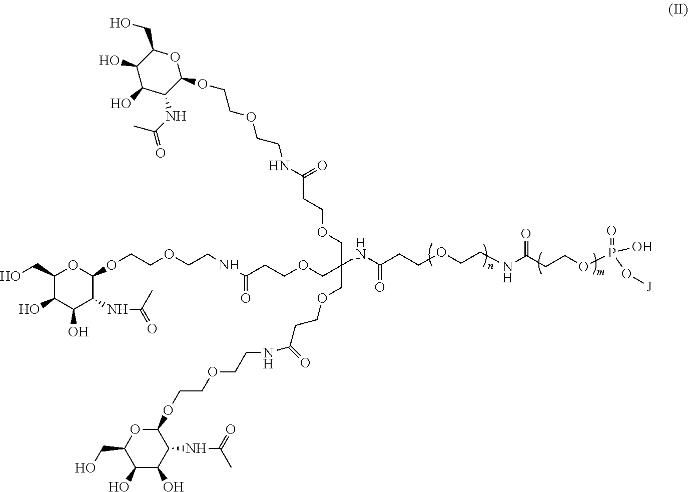
wherein
J is an oligonucleotide;
n is selected from any value from 1 to 10; and
m is selected from any value from 1 to 10.

28. The compound or salt of claim 27, wherein n is 2.

29. The compound or salt of claim 27, wherein m is 4.

30. The compound of claim 1, wherein the oligonucleotide (J) is attached at a 5′ end or a 3′ end of the oligonucleotide.

31. The compound of claim 1, wherein the oligonucleotide comprises DNA.

32. The compound of claim 1, wherein the oligonucleotide comprises RNA.

33. The compound of claim 1, wherein the oligonucleotide comprises one or more modified internucleoside linkages.

34. The compound of claim 33, wherein the one or more modified internucleoside linkages comprise alkylphosphonate, phosphorothioate, methylphosphonate, phosphorodithioate, alkylphosphonothioate, phosphoramidate, carbamate, carbonate, phosphate triester, acetamidate, or carboxymethyl ester, or a combination thereof.

35. The compound of claim 1, wherein the oligonucleotide comprises 1, 2, 3, 4, 5, 6, 7, 8, 9, 10, 11, 12, 13, 14, 15, 16, 17, 18, 19, or 20 modified internucleoside linkages.

36. The compound of claim 1, wherein the oligonucleotide comprises one or more modified nucleosides.

37. The compound of claim 36, wherein the one or more modified nucleosides comprise a locked nucleic acid (LNA), hexitol nucleic acid (HLA), cyclohexene nucleic acid (CeNA), 2′-methoxyethyl, 2′-O-alkyl, 2′-O-allyl, 2′-O-allyl, 2′-fluoro, or 2′-deoxy, or a combination thereof.

38. The compound of claim 36, wherein the one or more modified nucleosides comprise a 2′,4′ constrained ethyl nucleoside, a 2′-O-methyl nucleoside, 2′-deoxyfluoro nucleoside, 2′-O—N-methylacetamido (2′-O-NMA) nucleoside, a 2′-O-dimethylaminoethoxyethyl (2′-O-DMAEOE) nucleoside, 2′-O-aminopropyl (2′-O-AP) nucleoside, 2′-ara-F, 2′fluoro, or 2′ O-alkyl, or a combination thereof.

39. The compound of claim 1, wherein the oligonucleotide comprises 1, 2, 3, 4, 5, 6, 7, 8, 9, 10, 11, 12, 13, 14, 15, 16, 17, 18, 19, 20, 21, or more modified nucleosides.

40. The compound of claim 1, wherein the oligonucleotide comprises a lipid attached at a 3′ or 5′ terminus of the oligonucleotide.

41. The compound of claim 40, wherein the lipid comprises cholesterol, myristoyl, palmitoyl, stearoyl, lithocholoyl, docosanoyl, docosahexaenoyl, myristyl, palmityl stearyl, or α-tocopherol, or a combination thereof.

42. The compound of claim 1, wherein the oligonucleotide comprises an arginine-glycine-aspartic acid (RGD) peptide attached at a 3′ or 5′ terminus of the oligonucleotide.

43. The compound of claim 42, wherein the RGD peptide comprises Cyclo(-Arg-Gly-Asp-D-Phe-Cys), Cyclo(-Arg-Gly-Asp-D-Phe-Lys), Cyclo(-Arg-Gly-Asp-D-Phe-azido), an amino benzoic acid derived RGD, or a combination thereof.

44. The compound of claim 1, wherein the oligonucleotide comprises a small interfering RNA (siRNA) comprising a sense strand and an antisense strand.

45. The compound of claim 44, wherein the sense strand is 12-30 nucleosides in length.

46. The compound of claim 44, wherein the antisense strand is 12-30 nucleosides in length.

47. The compound of claim 44, wherein the sense strand and the antisense strand form a double-stranded RNA duplex.

48. The compound of claim 47, wherein a first base pair of the double-stranded RNA duplex is an AU base pair.

49. The compound of claim 44, wherein the sense strand or the antisense strand comprises a 3′ overhang.

50. The compound of claim 49, wherein the 3′ overhang comprises 1, 2, or more nucleosides.

51. The compound of claim 44, wherein the sense strand comprises any one of modification patterns 1 S to 6S, or 1S #2 to 6S #2.

52. The compound of claim 44, wherein the antisense strand comprises any one of modification patterns 1AS to 9AS.

53. The compound of claim 1, wherein the oligonucleotide comprises an antisense oligonucleotide (ASO).

54. The compound of claim 53, wherein the ASO is 12-30 nucleosides in length.

55. The compound of claim 53, wherein the ASO comprises modification pattern ASO1.

56. The compound of claim 1, wherein the compound binds to an asialoglycoprotein receptor.

57. The compound of claim 1, wherein the compound targets a hepatocyte.

58. A pharmaceutical composition comprising the compound of claim 1, and a pharmaceutically acceptable carrier, excipient, or diluent.

59. The pharmaceutical composition of claim 58, wherein the pharmaceutical composition is sterile.

60. The pharmaceutical composition of claim 58, wherein the pharmaceutical composition comprises a pharmaceutically acceptable carrier.

61. The pharmaceutical composition of claim 60, wherein the pharmaceutically acceptable carrier comprises water, a buffer, or a saline solution.

62. The pharmaceutical composition of claim 58, wherein the oligonucleotide targets a target mRNA and when administered to a subject in an effective amount decreases the target mRNA or a target protein by at least 10%.

63. A method of decreasing a target mRNA or target protein in a subject in need thereof, comprising administering an effective amount of the pharmaceutical composition of claim 58 to the subject.

64. The method of claim 63, wherein the effective amount decreases a measurement of the target mRNA or target protein in the subject, relative to a baseline target mRNA or target protein measurement.

65. The method of claim 63, wherein the effective amount treats a disorder in the subject.

66. The method of claim 65, wherein the effective amount decreases a measurement of a symptom or parameter related to the disorder in the subject, relative to a baseline symptom or parameter measurement.

67. The method of claim 65, wherein the disorder comprises a metabolic disorder.

68. The method of claim 65, wherein the disorder comprises a liver disorder.


SEQUENCE LISTING (28 sequences)

SEQ ID NO: 1 - RNA, 21 nt, Artificial Sequence
  Description of Artificial Sequence Synthetic oligonucleotide
  modified_base (1)..(1): 2′ fluoro-modified nucleoside
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′ fluoro-modified nucleoside
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′ fluoro-modified nucleoside
  modified_base (12)..(12): 2′O-methyl or 2′O-methoxyethyl-modified nucleo side
  modified_base (13)..(13): 2′ fluoro-modified nucleoside
  modified_base (14)..(14): 2′O-methyl or 2′O-methoxyethyl-modified nucleo side
  modified_base (15)..(15): 2′ fluoro-modified nucleoside
  modified_base (16)..(16): 2′O-methyl or 2′O-methoxyethyl-modified nucleo side
  modified_base (17)..(17): 2′ fluoro-modified nucleoside
  modified_base (18)..(18): 2′O-methyl or 2′O-methoxyethyl-modified nucleo side
  modified_base (19)..(19): 2′ fluoro-modified nucleoside
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 2 - RNA, 21 nt, Artificial Sequence
  Description of Artificial Sequence Synthetic oligonucleotide
  modified_base (1)..(1): 2′ fluoro-modified nucleoside
  modified_base (2)..(2): 2′O-methyl or 2′O-methoxyethyl-modified nucleo side
  modified_base (3)..(3): 2′ fluoro-modified nucleoside
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′ fluoro-modified nucleoside
  modified_base (12)..(12): 2′O-methyl or 2′O-methoxyethyl-modified nucleo side
  modified_base (13)..(13): 2′ fluoro-modified nucleoside
  modified_base (14)..(14): 2′O-methyl or 2′O-methoxyethyl-modified nucleo side
  modified_base (15)..(15): 2′ fluoro-modified nucleoside
  modified_base (16)..(16): 2′O-methyl or 2′O-methoxyethyl-modified nucleo side
  modified_base (17)..(17): 2′ fluoro-modified nucleoside
  modified_base (18)..(18): 2′O-methyl or 2′O-methoxyethyl-modified nucleo side
  modified_base (19)..(19): 2′ fluoro-modified nucleoside
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 3 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 4 - RNA, 21 nt, Artificial Sequence
  Description of Artificial Sequence Synthetic oligonucleotide
  modified_base (1)..(1): 2′O-methyl or 2′O-methoxyethyl-modified nucleo side
  modified_base (2)..(2): 2′O-methyl or 2′O-methoxyethyl-modified nucleo side
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 5 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′O-methyl or 2′O-methoxyethyl-modified nucleo side
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 6 - RNA, 21 nt, Artificial Sequence
  Description of Artificial Sequence Synthetic oligonucleotide
  modified_base (1)..(1): 2′O-methyl or 2′O-methoxyethyl-modified nucleo side
  modified_base (2)..(2): 2′O-methyl or 2′O-methoxyethyl-modified nucleo side
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′O-methyl or 2′O-methoxyethyl-modified nucleo side
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 7 - RNA, 22 nt, Artificial Sequence
  Description of Artificial Sequence Synthetic oligonucleotide
  modified_base (1)..(1): 2′ fluoro-modified nucleoside
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′ fluoro-modified nucleoside
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′ fluoro-modified nucleoside
  modified_base (12)..(12): 2′O-methyl or 2′O-methoxyethyl-modified nucleo side
  modified_base (13)..(13): 2′ fluoro-modified nucleoside
  modified_base (14)..(14): 2′O-methyl or 2′O-methoxyethyl-modified nucleo side
  modified_base (15)..(15): 2′ fluoro-modified nucleoside
  modified_base (16)..(16): 2′O-methyl or 2′O-methoxyethyl-modified nucleo side
  modified_base (17)..(17): 2′ fluoro-modified nucleoside
  modified_base (18)..(18): 2′O-methyl or 2′O-methoxyethyl-modified nucleo side
  modified_base (19)..(19): 2′ fluoro-modified nucleoside
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
  modified_base (22)..(22): a, c, u, g, unknown or other
  See specification as filed for detailed description of substitutions and preferred embodiments
nnnnnnnnnnnnnnnnnnnnnn

SEQ ID NO: 8 - RNA, 22 nt, Artificial Sequence
  Description of Artificial Sequence Synthetic oligonucleotide
  modified_base (1)..(1): 2′ fluoro-modified nucleoside
  modified_base (2)..(2): 2′O-methyl or 2′O-methoxyethyl-modified nucleo side
  modified_base (3)..(3): 2′ fluoro-modified nucleoside
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′ fluoro-modified nucleoside
  modified_base (12)..(12): 2′O-methyl or 2′O-methoxyethyl-modified nucleo side
  modified_base (13)..(13): 2′ fluoro-modified nucleoside
  modified_base (14)..(14): 2′O-methyl or 2′O-methoxyethyl-modified nucleo side
  modified_base (15)..(15): 2′ fluoro-modified nucleoside
  modified_base (16)..(16): 2′O-methyl or 2′O-methoxyethyl-modified nucleo side
  modified_base (17)..(17): 2′ fluoro-modified nucleoside
  modified_base (18)..(18): 2′O-methyl or 2′O-methoxyethyl-modified nucleo side
  modified_base (19)..(19): 2′ fluoro-modified nucleoside
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
  modified_base (22)..(22): a, c, u, g, unknown or other
  See specification as filed for detailed description of substitutions and preferred embodiments
nnnnnnnnnnnnnnnnnnnnnn

SEQ ID NO: 9 - RNA, 22 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
  modified_base (22)..(22): a, c, u, g, unknown or other
  See specification as filed for detailed description of substitutions and preferred embodiments
nnnnnnnnnnnnnnnnnnnnnn

SEQ ID NO: 10 - RNA, 22 nt, Artificial Sequence
  Description of Artificial Sequence Synthetic oligonucleotide
  modified_base (1)..(1): 2′O-methyl or 2′O-methoxyethyl-modified nucleo side
  modified_base (2)..(2): 2′O-methyl or 2′O-methoxyethyl-modified nucleo side
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
  modified_base (22)..(22): a, c, u, g, unknown or other
  See specification as filed for detailed description of substitutions and preferred embodiments
nnnnnnnnnnnnnnnnnnnnnn

SEQ ID NO: 11 - RNA, 21 nt, Artificial Sequence
  Description of Artificial Sequence Synthetic oligonucleotide
  modified_base (1)..(1): 2′ fluoro-modified nucleoside
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′ fluoro-modified nucleoside
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′O-methyl or 2′O-methoxyethyl-modified nucleo side
  modified_base (9)..(9): 2′ fluoro-modified nucleoside
  modified_base (10)..(10): 2′O-methyl or 2′O-methoxyethyl-modified nucleo side
  modified_base (11)..(11): 2′ fluoro-modified nucleoside
  modified_base (12)..(12): 2′O-methyl or 2′O-methoxyethyl-modified nucleo side
  modified_base (13)..(13): 2′ fluoro-modified nucleoside
  modified_base (14)..(14): 2′O-methyl or 2′O-methoxyethyl-modified nucleo side
  modified_base (15)..(15): 2′ fluoro-modified nucleoside
  modified_base (16)..(16): 2′O-methyl or 2′O-methoxyethyl-modified nucleo side
  modified_base (17)..(17): 2′ fluoro-modified nucleoside
  modified_base (18)..(18): 2′O-methyl or 2′O-methoxyethyl-modified nucleo side
  modified_base (19)..(19): 2′ fluoro-modified nucleoside
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 12 - RNA, 21 nt, Artificial Sequence
  Description of Artificial Sequence Synthetic oligonucleotide
  modified_base (1)..(1): 2′ fluoro-modified nucleoside
  modified_base (2)..(2): 2′O-methyl or 2′O-methoxyethyl-modified nucleo side
  modified_base (3)..(3): 2′ fluoro-modified nucleoside
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′O-methyl or 2′O-methoxyethyl-modified nucleo side
  modified_base (9)..(9): 2′ fluoro-modified nucleoside
  modified_base (10)..(10): 2′O-methyl or 2′O-methoxyethyl-modified nucleo side
  modified_base (11)..(11): 2′ fluoro-modified nucleoside
  modified_base (12)..(12): 2′O-methyl or 2′O-methoxyethyl-modified nucleo side
  modified_base (13)..(13): 2′ fluoro-modified nucleoside
  modified_base (14)..(14): 2′O-methyl or 2′O-methoxyethyl-modified nucleo side
  modified_base (15)..(15): 2′ fluoro-modified nucleoside
  modified_base (16)..(16): 2′O-methyl or 2′O-methoxyethyl-modified nucleo side
  modified_base (17)..(17): 2′ fluoro-modified nucleoside
  modified_base (18)..(18): 2′O-methyl or 2′O-methoxyethyl-modified nucleo side
  modified_base (19)..(19): 2′ fluoro-modified nucleoside
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 13 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′ fluoro-modified nucleoside
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′ fluoro-modified nucleoside
  modified_base (9)..(9): 2′O-methyl or 2′O-methoxyethyl-modified nucleo side
  modified_base (10)..(10): 2′ fluoro-modified nucleoside
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′ fluoro-modified nucleoside
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 14 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 15 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′O-methyl or 2′O-methoxyethyl-modified nucleo side
  modified_base (9)..(9): 2′O-methyl or 2′O-methoxyethyl-modified nucleo side
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 16 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′ fluoro-modified nucleoside
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′O-methyl or 2′O-methoxyethyl-modified nucleo side
  modified_base (9)..(9): 2′O-methyl or 2′O-methoxyethyl-modified nucleo side
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 17 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′ fluoro-modified nucleoside
  modified_base (9)..(9): 2′O-methyl or 2′O-methoxyethyl-modified nucleo side
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 18 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′ fluoro-modified nucleoside
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′ fluoro-modified nucleoside
  modified_base (9)..(9): 2′O-methyl or 2′O-methoxyethyl-modified nucleo side
  modified_base (10)..(10): 2′ fluoro-modified nucleoside
  modified_base (11)..(11): 2′O-methyl or 2′O-methoxyethyl-modified nucleo side
  modified_base (12)..(12): 2′ fluoro-modified nucleoside
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′ fluoro-modified nucleoside
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 19 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′ fluoro-modified nucleoside
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 20 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′O-methyl or 2′O-methoxyethyl-modified nucleo side
  modified_base (6)..(6): 2′O-methyl or 2′O-methoxyethyl-modified nucleo side
  modified_base (7)..(7): 2′O-methyl or 2′O-methoxyethyl-modified nucleo side
  modified_base (8)..(8): 2′O-methyl or 2′O-methoxyethyl-modified nucleo side
  modified_base (9)..(9): 2′O-methyl or 2′O-methoxyethyl-modified nucleo side
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 21 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′O-methyl or 2′O-methoxyethyl-modified nucleo side
  modified_base (9)..(9): 2′O-methyl or 2′O-methoxyethyl-modified nucleo side
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′ fluoro-modified nucleoside
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
nnnnnnnnnnnnnnnnnnnnn

SEQ ID NO: 22 - DNA, 20 bp, Artificial Sequence
  Description of Artificial Sequence Synthetic oligonucleotide
  Description of Combined DNA/RNA Molecule Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′O-methyl or 2′O-methoxyethyl-modified nucleo side
  misc_feature (3)..(4): Phosphorothioate linkage
  modified_base (4)..(4): 2′O-methyl or 2′O-methoxyethyl-modified nucleo side
  misc_feature (4)..(5): Phosphorothioate linkage
  modified_base (5)..(5): 2′O-methyl or 2′O-methoxyethyl-modified nucleo side
  misc_feature (5)..(6): Phosphorothioate linkage
  modified_base (6)..(6): Any deoxynucleotide
  misc_feature (6)..(7): Phosphorothioate linkage
  modified_base (7)..(7): Any deoxynucleotide
  misc_feature (7)..(8): Phosphorothioate linkage
  modified_base (8)..(8): Any deoxynucleotide
  misc_feature (8)..(9): Phosphorothioate linkage
  modified_base (9)..(9): Any deoxynucleotide
  misc_feature (9)..(10): Phosphorothioate linkage
  modified_base (10)..(10): Any deoxynucleotide
  misc_feature (10)..(11): Phosphorothioate linkage
  modified_base (11)..(11): Any deoxynucleotide
  misc_feature (11)..(12): Phosphorothioate linkage
  modified_base (12)..(12): Any deoxynucleotide
  misc_feature (12)..(13): Phosphorothioate linkage
  modified_base (13)..(13): Any deoxynucleotide
  misc_feature (13)..(14): Phosphorothioate linkage
  modified_base (14)..(14): Any deoxynucleotide
  misc_feature (14)..(15): Phosphorothioate linkage
  modified_base (15)..(15): Any deoxynucleotide
  misc_feature (15)..(16): Phosphorothioate linkage
  modified_base (16)..(16): 2′O-methyl or 2′O-methoxyethyl-modified nucleo side
  misc_feature (16)..(17): Phosphorothioate linkage
  modified_base (17)..(17): 2′O-methyl or 2′O-methoxyethyl-modified nucleo side
  misc_feature (17)..(18): Phosphorothioate linkage
  modified_base (18)..(18): 2′O-methyl or 2′O-methoxyethyl-modified nucleo side
  misc_feature (18)..(19): Phosphorothioate linkage
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
nnnnnnnnnnnnnnnnnnnn

SEQ ID NO: 23 - RNA, 19 nt, Artificial Sequence
  Description of Artificial Sequence Synthetic oligonucleotide
ggacaauacuuccacucua

SEQ ID NO: 24 - RNA, 21 nt, Artificial Sequence
  Description of Artificial Sequence Synthetic oligonucleotide
  modified_base (1)..(1): 2′O-methyl or 2′O-methoxyethyl-modified nucleo side
  modified_base (2)..(2): 2′O-methyl or 2′O-methoxyethyl-modified nucleo side
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
ggacaauacuuccacucuauu

SEQ ID NO: 25 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
ggacaauacuuccacucuauu

SEQ ID NO: 26 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′O-methyl or 2′O-methoxyethyl-modified nucleo side
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′ fluoro-modified nucleoside
  modified_base (6)..(6): 2′O-methyl or 2′O-methoxyethyl-modified nucleo side
  modified_base (7)..(7): 2′ fluoro-modified nucleoside
  modified_base (8)..(8): 2′ fluoro-modified nucleoside
  modified_base (9)..(9): 2′ fluoro-modified nucleoside
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′O-methyl or 2′O-methoxyethyl-modified nucleo side
  modified_base (15)..(15): 2′O-methyl or 2′O-methoxyethyl-modified nucleo side
  modified_base (16)..(16): 2′O-methyl or 2′O-methoxyethyl-modified nucleo side
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
ggacaauacuuccacucuauu

SEQ ID NO: 27 - RNA, 19 nt, Artificial Sequence
  Description of Artificial Sequence Synthetic oligonucleotide
uagaguggaaguauugucc

SEQ ID NO: 28 - RNA, 21 nt, Artificial Sequence
  Description of Artificial Sequence Synthetic oligonucleotide
  modified_base (1)..(1): 2′O-methyl or 2′O-methoxyethyl-modified nucleo side
  misc_feature (1)..(2): Phosphorothioate linkage
  modified_base (2)..(2): 2′ fluoro-modified nucleoside
  misc_feature (2)..(3): Phosphorothioate linkage
  modified_base (3)..(3): 2′O-methyl or 2′O-methoxyethyl-modified nucleo side
  modified_base (4)..(4): 2′O-methyl or 2′O-methoxyethyl-modified nucleo side
  modified_base (5)..(5): 2′O-methyl or 2′O-methoxyethyl-modified nucleo side
  modified_base (6)..(6): 2′ fluoro-modified nucleoside
  modified_base (7)..(7): 2′O-methyl or 2′O-methoxyethyl-modified nucleo side
  modified_base (8)..(8): 2′O-methyl or 2′O-methoxyethyl-modified nucleo side
  modified_base (9)..(9): 2′O-methyl or 2′O-methoxyethyl-modified nucleo side
  modified_base (10)..(10): 2′O-methyl or 2′O-methoxyethyl-modified nucleo side
  modified_base (11)..(11): 2′O-methyl or 2′O-methoxyethyl-modified nucleo side
  modified_base (12)..(12): 2′O-methyl or 2′O-methoxyethyl-modified nucleo side
  modified_base (13)..(13): 2′O-methyl or 2′O-methoxyethyl-modified nucleo side
  modified_base (14)..(14): 2′ fluoro-modified nucleoside
  modified_base (15)..(15): 2′O-methyl or 2′O-methoxyethyl-modified nucleo side
  modified_base (16)..(16): 2′ fluoro-modified nucleoside
  modified_base (17)..(17): 2′O-methyl or 2′O-methoxyethyl-modified nucleo side
  modified_base (18)..(18): 2′O-methyl or 2′O-methoxyethyl-modified nucleo side
  modified_base (19)..(19): 2′O-methyl or 2′O-methoxyethyl-modified nucleo side
  misc_feature (19)..(20): Phosphorothioate linkage
  modified_base (20)..(20): 2′O-methyl or 2′O-methoxyethyl-modified nucleo side
  misc_feature (20)..(21): Phosphorothioate linkage
  modified_base (21)..(21): 2′O-methyl or 2′O-methoxyethyl-modified nucleo side
uagaguggaaguauuguccuu